SEQUENCE LISTING (30 sequences)

SEQ ID NO: 1 - DNA, 20 bp, Artificial Sequence
  Description of Artificial Sequence Synthetic primer
ccaccagtgatttctgctca

SEQ ID NO: 2 - DNA, 20 bp, Artificial Sequence
  Description of Artificial Sequence Synthetic primer
agtacgagccatgcaagacc

SEQ ID NO: 3 - DNA, 19 bp, Artificial Sequence
  Description of Artificial Sequence Synthetic primer
tccgggctgccacgaccaa

SEQ ID NO: 4 - DNA, 19 bp, Artificial Sequence
  Description of Artificial Sequence Synthetic primer
ggcgcggcaacaccatttt

SEQ ID NO: 5 - DNA, 18 bp, Artificial Sequence
  Description of Artificial Sequence Synthetic primer
tgtaaaacgacggccagt

SEQ ID NO: 6 - DNA, 17 bp, Artificial Sequence
  Description of Artificial Sequence Synthetic primer
caggaaacagctatgac

SEQ ID NO: 7 - DNA, 19 bp, Artificial Sequence
  Description of Artificial Sequence Synthetic primer
aagctggccccagactttc

SEQ ID NO: 8 - DNA, 20 bp, Artificial Sequence
  Description of Artificial Sequence Synthetic primer
gcataaggcaacccttagca

SEQ ID NO: 9 - DNA, 20 bp, Artificial Sequence
  Description of Artificial Sequence Synthetic primer
gccctatgaggtaggggcta

SEQ ID NO: 10 - DNA, 20 bp, Artificial Sequence
  Description of Artificial Sequence Synthetic primer
caccaggacatgcacagcta

SEQ ID NO: 11 - DNA, 20 bp, Artificial Sequence
  Description of Artificial Sequence Synthetic primer
agcattgccctgttgtgttc

SEQ ID NO: 12 - DNA, 20 bp, Artificial Sequence
  Description of Artificial Sequence Synthetic primer
ctatgccttgatggctggag

SEQ ID NO: 13 - DNA, 20 bp, Artificial Sequence
  Description of Artificial Sequence Synthetic primer
aggctctgtcccagttaccg

SEQ ID NO: 14 - DNA, 20 bp, Artificial Sequence
  Description of Artificial Sequence Synthetic primer
tgtagccaccctggactcag

SEQ ID NO: 15 - DNA, 20 bp, Artificial Sequence
  Description of Artificial Sequence Synthetic primer
ccatgagaatcacgcagtgg

SEQ ID NO: 16 - DNA, 20 bp, Artificial Sequence
  Description of Artificial Sequence Synthetic primer
ctgtgaaggcctcagctcct

SEQ ID NO: 17 - DNA, 20 bp, Artificial Sequence
  Description of Artificial Sequence Synthetic primer
gctccagggtggaatctctc

SEQ ID NO: 18 - DNA, 20 bp, Artificial Sequence
  Description of Artificial Sequence Synthetic primer
acctgaaggagatgccaagg

SEQ ID NO: 19 - DNA, 20 bp, Artificial Sequence
  Description of Artificial Sequence Synthetic primer
caacagtttgaggcctgagc

SEQ ID NO: 20 - DNA, 20 bp, Artificial Sequence
  Description of Artificial Sequence Synthetic primer
gcttgacaaccatgctccat

SEQ ID NO: 21 - DNA, 20 bp, Artificial Sequence
  Description of Artificial Sequence Synthetic primer
agccacaaaggtggcctaaa

SEQ ID NO: 22 - DNA, 20 bp, Artificial Sequence
  Description of Artificial Sequence Synthetic primer
ggacacaggtcggatttgaa

SEQ ID NO: 23 - DNA, 21 bp, Artificial Sequence
  Description of Artificial Sequence Synthetic primer
ccagcacggtaccgatagttc

SEQ ID NO: 24 - DNA, 20 bp, Artificial Sequence
  Description of Artificial Sequence Synthetic primer
gaaccttggtcatggctttg

SEQ ID NO: 25 - DNA, 20 bp, Artificial Sequence
  Description of Artificial Sequence Synthetic primer
ccccttctcagagggagttg

SEQ ID NO: 26 - DNA, 20 bp, Artificial Sequence
  Description of Artificial Sequence Synthetic primer
acctgctcctgtgcattgac

SEQ ID NO: 27 - DNA, 20 bp, Artificial Sequence
  Description of Artificial Sequence Synthetic primer
ggctcctgtggctcctactt

SEQ ID NO: 28 - DNA, 24 bp, Artificial Sequence
  Description of Artificial Sequence Synthetic primer
cagagaagaaggaagagagagcaa

SEQ ID NO: 29 - DNA, 20 bp, Artificial Sequence
  Description of Artificial Sequence Synthetic primer
cactgtcagggggacaaaga

SEQ ID NO: 30 - DNA, 20 bp, Artificial Sequence
  Description of Artificial Sequence Synthetic primer
cagacacttggcccacagac

(19) United States
(12) Patent Application Publication
(10) Pub. No.: US 2012/0010229 A1
(43) Pub. Date: Jan. 12, 2012

(54) THERAPEUTIC REGIMENS FOR HEDGEHOG-ASSOCIATED CANCERS

(76) Inventors: John R. MacDougall, Hingham, MA (US); Karen J. McGovern, Groton, MA (US)

(21) Appl. No.: 13/179,404

(22) Filed: Jul. 8, 2011

Related U.S. Application Data
(60) Provisional application No. 61/362,568, filed on Jul. 8, 2010.
(60) Provisional application No. 61/393,347, filed on Oct. 14, 2010.
(60) Provisional application No. 61/471,028, filed on Apr. 1, 2011.

(51) Int. Cl. A61K 31/4355; A61P 35/00; C07D 491/04

(52) U.S. Cl. 514/278; 546/15

(57) ABSTRACT

Provided herein are methods, therapeutic regimens, and kits that optimize the benefits of hedgehog inhibition for cancer therapy.

### CROSS REFERENCE TO RELATED APPLICATIONS

This application claims the benefit of priority to U.S. Provisional Application Ser. No. 61/362,568, filed Jul. 8, 2010; U.S. Provisional Application Ser. No. 61/393,347, filed Oct. 14, 2010; and U.S. Provisional Application Ser. No. 61/471,028, filed Apr. 1, 2011. The contents of all of the aforesaid applications are hereby incorporated by reference in their entirety. A PCT patent application entitled “Therapeutic Regimens for Hedgehog-Associated Cancers,” filed Jul. 8, 2011 with the U.S. Receiving Office and designating attorney docket number I2041-7004WO is also incorporated by reference.

### SEQUENCE LISTING

The instant application contains a Sequence Listing which has been submitted in ASCII format via EFS-Web and is hereby incorporated by reference in its entirety. Said ASCII copy, created on Jul. 8, 2011, is named I204174W.txt and is 7,123 bytes in size.

### BACKGROUND

Hedgehog signaling plays a role in many stages of development, especially in formation of left-right symmetry. Loss or reduction of hedgehog signaling leads to multiple developmental deficits and malformations, one of the most striking of which is cyclopia.

Many cancers and proliferative conditions have been shown to depend on the hedgehog pathway. It has been reported that activating hedgehog pathway mutations occur in sporadic basal cell carcinoma (Xie et al. (1998) Nature 391: 90-2) and primitive neuroectodermal tumors of the central nervous system (Reifenberger et al. (1998) Cancer Res 58: 1798-803). Uncontrolled activation of the hedgehog pathway has also been shown in numerous cancer types such as GI tract cancers including pancreatic, esophageal, gastric cancer (Berman et al. (2003) Nature 425: 846-51; Thayer et al. (2003) Nature 425: 851-56), lung cancer (Watkins et al. (2003) Nature 422: 313-317), prostate cancer (Karhadkar et al. (2004) Nature 431: 707-12; Sheng et al. (2004) Molecular Cancer 3: 29-42; Fan et al. (2004) Endocrinology 145: 3961-70), breast cancer (Kubo et al. (2004) Cancer Research 64: 6071-74; Lewis et al. (2004) Journal of Mammary Gland Biology and Neoplasia 2: 165-181) and hepatocellular cancer (Sicklick et al. (2005) ASCO conference; Mohini et al. (2005) AACR conference).

Research to date has focused on the elucidation of hedgehog pathway biology and the discovery of new hedgehog pathway inhibitors. Progress toward the development of clinical candidates has been hampered by a poor understanding of the timing and dosing regimen required to optimally treat hedgehog-associated disorders, in particular, hedgehog-associated cancers. Therefore, the need still exists for developing therapeutic regimens that optimize the benefits of hedgehog inhibition.

### SUMMARY

Applicants have discovered that a hedgehog inhibitor (e.g., IPI-926) can be used effectively following cyto-reductive chemotherapy. In one embodiment, the hedgehog inhibitor is administered either concurrently with cancer therapy (e.g., having at least some period of overlap between the cancer therapy treatment regimen and the administration of the hedgehog inhibitor), or without a substantial delay after cessation of cancer therapy. In related embodiments, the hedgehog inhibitor (e.g., IPI-926) has been shown to be effective as cytoreductive therapy to treat minimal residual disease, and/or as maintenance therapy, in a wide number of tumor types, including, but not limited to, ovarian cancer, prostate cancer and non-small cell lung cancer. In yet other embodiments, Applicants have shown that pre-treatment of a subject with a hedgehog inhibitor (e.g., IPI-926) reduces the formation and growth of metastatic tumors, leading to a reduction in tumor burden and increased survival. In some embodiments, the hedgehog inhibitor (e.g., IPI-926) can reduce the tumor ability to reestablish itself after therapy or establish anew. In other embodiments, the hedgehog inhibitor (e.g., IPI-926) can inhibit or reduce one or more of: the stroma to which metastatic cells seed; angiogenic mechanisms associated with solid tumor growth and maintenance; and/or minimal residual disease. Accordingly, the present invention relates to new treatment regimens, treatment schedules, methods and kits that optimize the benefits of hedgehog inhibition for cancer therapy.

Accordingly, in one aspect, the invention features a method of treating (e.g., reducing or inhibiting the growth or re-growth of; reducing or inhibiting minimal residual disease of) a hedgehog-associated cancer, e.g., one or more ligand-dependent and/or ligand-independent cancers or tumors. The method includes administering to a subject a hedgehog inhibitor (e.g., one or more hedgehog inhibitors as described herein), in an amount sufficient to reduce or inhibit the tumor cell growth or re-growth, and/or treat the cancer or the minimal residual disease, in the subject. In one embodiment, the hedgehog inhibitor is administered at least partially concurrently with, or without a substantially delay after cessation of, a cancer therapy (e.g., a primary cancer therapy that includes one or more anti-cancer agents, radiation therapy and/or surgery). For example, the method includes: administering the hedgehog inhibitor prior to cessation of the cancer therapy (e.g., after initiation, but prior to cessation, of the cancer therapy; having at least some period of overlap between the treatment regimen and the administration of the hedgehog inhibitor; for example, at least 1, 2, 3, 4, 5, 10, 15, 24, 36, or 48 hours; at least 1, 2, 3, 4, 5, 6, 7, 10, 14, or 20 days; at least 1, 2, 3, 4, 5, 6, 8, 10, or 12 months; prior to cessation of cancer therapy). In other embodiments, the method includes administering the hedgehog inhibitor without a substantial delay after cessation of a treatment regimen (e.g., simultaneously with, or less than 15, 10, 8, 6, 5, 4, 3 days, or less than 144, 120, 100, 90, 72, 60, 48, 36, 24, 14, 10, 9, 8, 7, 6, 5, 4, 3, 2, or 1 hour after cessation of the cancer therapy).

In one embodiment, the hedgehog inhibitor is administered to a subject (e.g., a cancer patient) as maintenance therapy (e.g., as a prolonged or extended therapy after cessation of another cancer treatment). For example, the hedgehog inhibitor is administered after cessation of another cancer therapy (e.g., a primary cancer therapy one or more therapeutic agents, radiation therapy and/or surgery). In one embodiment, the hedgehog inhibitor is administered at a diminished dose from a first line therapeutic dose (e.g., a therapeutic dose administered to a subject who has not been previously administered another drug intended to treat the cancer). In one embodiment, the hedgehog inhibitor is administered at a dose that is less than 10%, 20%, 30%, 40%, 50%, 60%, 70%, 80%, 90% or 99% of the first line therapeutic dose). In embodiments, the hedgehog inhibitor delays the re-growth or recurrence of the cancer or tumor by at least 1, 5, 10, 15, 20, 30, 50, 100 days; 3, 4, 5, 6, 12, 18 months; or 1, 2, 3, 4, or at least 5 years, compared to an untreated subject. In other embodiments, the size of the tumor re-growth is reduced by at least 10%, 20%, 30%, 40%, 50%, 60%, 65%, 70%, 80%, or at least 90%, compared to an untreated subject. Treatment with the hedgehog inhibitor can continue as long as clinically necessary (e.g., for 1, 5, 10, 15, 20, 25, 30 days; 1, 2, 4, 6, 8, 12 months; or 1, 1.5, 2, 2.5, 3, 5 years or longer). In one embodiment, the hedgehog inhibitor is administered chronically as a single agent. In other embodiments, the hedgehog inhibitor is administered in a pre-determined schedule (e.g., continuous therapy followed by one or more of: drug free intervals, combinations with other cancer therapies, or alternating with other cancer therapies).

In certain embodiments, the hedgehog inhibitor is administered to a cancer patient after cessation of another cancer therapy (e.g., a primary cancer therapy), such as chemotherapy, radiation therapy and/or surgery. In certain embodiments, the subject has minimal residual disease after the primary cancer therapy (e.g., chemotherapy, radiation therapy and/or surgery). For example, the subject is a patient with SCLC previously treated with a primary treatment for SCLC (e.g., etoposide and/or cisplatin); the subject is a patient with NSCLC previously treated with a tyrosine kinase inhibitor (e.g., gefitinib); the subject is a patient with ovarian cancer previously treated with a taxol and/or carboplatin.

The subject can be a cancer patient substantially or completely in remission from a cancer (e.g., a cancer chosen from one or more of: lung cancer (e.g., small cell lung cancer or non-small cell lung cancer), pancreatic cancer, prostate cancer, bladder cancer, ovarian cancer, breast cancer, colon cancer, biliary cancer, myelofibrotic cancer, medulloblastoma, multiple myeloma, acute myelogenous leukemia (AML), chronic myelogenous leukemia (CML), acute lymphocytic leukemia (ALL), and neuroendocrine cancer).

In a related aspect, the invention features a method of preventing, or reducing, a relapse in a hedgehog-associated cancer (e.g., one or more of ligand-dependent and/or ligand-independent cancers or tumors), in a subject (e.g., a cancer patient). The method includes administering a hedgehog inhibitor(s) as cytoreductive therapy to treat minimal residual disease, and/or as maintenance therapy (e.g., as a prolonged or extended therapy after cessation of another cancer treatment). For example, the hedgehog inhibitor(s) is administered after cessation of another cancer therapy, such as chemotherapy, radiation therapy and/or surgery.

In one embodiment, the hedgehog inhibitor(s) is administered at a diminished dose from a first line therapeutic dose (e.g., a therapeutic dose administered to a subject who has not been previously administered another drug intended to treat the cancer). In one embodiment, the hedgehog inhibitor(s) is administered at a dose that is less than 10%, 20%, 30%, 40%, 50%, 60%, 70%, 80%, or at least 90% of the first line therapeutic dose). In embodiments, the hedgehog inhibitor delays the re-growth or recurrence of the cancer or tumor by at least 1, 5, 10, 15, 20, 30, 50, 100 days; 3, 4, 5, 6, 12, 18 months; or 1, 2, 3, 4, or at least 5 years, compared to an untreated subject. In other embodiments, the size of the tumor re-growth is reduced by at least 10%, 20%, 30%, 40%, 50%, 60%, 65%, 70%, 80%, or at least 90%, compared to an untreated subject. Treatment with the hedgehog inhibitor can continue as long as clinically necessary (e.g., for 1, 5, 10, 15, 20, 25, 30 days; 1, 2, 4, 6, 8, 12 months; or 1, 1.5, 2, 2.5, 3, 5 years or longer). In one embodiment, the hedgehog inhibitor is administered chronically as a single agent. In other embodiments, the hedgehog inhibitor is administered in a pre-determined schedule (e.g., continuous therapy followed by one or more of: drug free intervals, combinations with other cancer therapies, or alternating with other cancer therapies).

In another aspect, the invention features a method to treat or prevent a metastasis or metastatic growth of a hedgehog associated cancer. The method includes administering to a subject (e.g., a cancer patient) one or more hedgehog inhibitors prior to detection of a metastatic lesion. In one embodiment, the subject has a localized cancer that is treated with one or more hedgehog inhibitors (e.g., IPI-926) to reduce the formation and growth of metastatic tumors, and/or increased survival.

In another aspect, the invention features a method of reducing minimal residual disease in a subject. For example, chemotherapy of patients with small cell lung cancer (SCLC) is often followed with prophylactic cranial irradiation (PCI). If no PCI is administered, many patients tend to develop brain metastasis (see Slotman, B. et al (2007) N Engl J Med 357(7): 664-672 and Patel, S. et al. (2009) Cancer 842-850). Administration of one or more of the hedgehog inhibitors disclosed herein can be used in lieu of PCI. Thus, the method includes administering one or more hedgehog inhibitors to a patient who has undergone another cancer therapy treatment regimen (e.g., treatment with one or more therapeutic agents and/or radiation and/or surgery), in an amount sufficient to reduce the minimal residual disease. In one embodiment, the subject is a patient (e.g., a patient with SCLC) who is undergoing or has undergone one or more of radiation, chemotherapy and/or surgery) and shows at least 10%, 20%, 30%, 40%, 50%, 60%, 70%, 80%, 90% or more tumor shrinkage. The method can further include the step of identifying the subject showing such tumor shrinkage. In one embodiment, the subject is administered one or more hedgehog inhibitors, instead of PCI (e.g., one or more hedgehog inhibitors replace PCI to prevent metastasis (e.g., brain metastasis)). In other embodiments, the subject is identified, or has, lung cancer (e.g., NSCLC or SCLC). In other embodiments, the subject is identified, or has, limited stage SCLC. In other embodiments, the subject is identified, or has, extensive SCLC. In other embodiments, the subject is identified, or has, prostate cancer. In other embodiments, the subject is identified, or has, ovarian cancer.

In yet another aspect, the invention features a method for treating (e.g., reducing or inhibiting the growth or re-growth of; reducing or inhibiting) a hedgehog-associated cancer or tumor, e.g., one or more ligand-dependent and/or ligand-independent cancers or tumors.

In one embodiment, the hedgehog-associated cancers or tumors are resistant (partially or completely resistant or refractory to another cancer therapy, referred to herein as “resistant tumor or cancer”). The method includes administering to a subject a hedgehog inhibitor(s) (e.g., a first hedgehog inhibitor as described herein (e.g., IPI-926) in an amount sufficient to reduce or inhibit the tumor cell growth or re-growth, and/or treat or prevent the cancer(s) or tumor(s), in the subject. In one embodiment, the tumor or cancer is a medulloblastoma.

In one embodiment, the tumor or cancer harbors a mutation that renders the tumor or cancer resistant to a hedgehog inhibitor (e.g., a second hedgehog inhibitor such as GDC-0449). For example, the cancer or tumor harbors one or more mutations in a hedgehog receptor (e.g., Smoothened or Patched). Mutations in Smoothened that confer resistance to GDC-0449 in medulloblastoma are described by Yauch, R. L. et al. (2009) Science 326: 572-574 Sciencexpress: 1-3 (10.1126/science. 1179386); Rudin, C. et al. (2009) New England J of Medicine 361-366 (10.1056/nejma0902903). In one embodiment, the cancer or tumor harbors one or more mutations at position 473 (e.g., a D473H substitution; a heterozygous G to C missense mutation at position 1637).

In other embodiments, the tumor or cancer overexpress one or more of GLI2, SHH. In one embodiment, the subject is a patient with a medulloblastoma having SHH overexpression.

The method can further include identifying a patient likely to develop resistance to a hedgehog inhibitor (e.g., a second hedgehog inhibitor such as GDC-0449). The method includes detecting the presence of one or more mutations in a hedgehog receptor. In one embodiment, one or more mutations detected are found at position 473 (e.g., a D473H substitution; a heterozygous G to C missense mutation at position 1637).

In another embodiment, the tumor or cancer shows increased expression or activity of a compensatory mechanism in response to hedgehog inhibition. For example, the tumor or cancer (e.g., a medulloblastoma) has increased expression and/or activity of the phosphoinositide 3-kinase (PI3K) pathway. In other embodiments, the tumor or cancer is a medulloblastoma that has SHH overexpression. In such embodiments, the hedgehog inhibitor (e.g., IPI-926) is administered in combination with a PI3K inhibitor. In one embodiment, the PI3K inhibitor is an inhibitor of delta and gamma isoforms of PI3K. Exemplary PI3K inhibitors that can be used in combination are described in, e.g., WO 09/088,990; WO 09/088,086; WO 2011/008302; WO 2010/036380; WO 2010/006086, WO 09/114,870, WO 05/113556; US 2009/0312310, US 2011/0046165. Additional PI3K inhibitors that can be used in combination with the hedgehog inhibitors, include but are not limited to, GSK 2126458, GDC-0980, GDC-0941, Sanofi XL147, XL756, XL147, PF-46915032, Novartis BEZ 235, BKM 120, CAL-101, CAL 263, SF1126 and PX-886. In one embodiment, the PI3K inhibitor is an isoquinolinone. In one embodiment, the PI3K inhibitor is INK1197 or a derivative thereof. In other embodiments, the PI3K inhibitor is INK1117 or a derivative thereof. The hedgehog inhibitor and the PI3K inhibitor can be administered simultaneously or sequentially as described herein. In certain embodiments, the inhibitors are administered in the same composition, or in different compositions, as described hereinbelow.

In yet other embodiments, the hedgehog inhibitor (e.g., one or more of the hedgehog inhibitors described herein) are administered in combination. For example, IPI-926 is administered in combination with other hedgehog inhibitors, e.g., GDC-0449.

In one embodiment, the tumor harboring the one or more mutations is a medulloblastoma. In certain embodiments, the one or more hedgehog inhibitors (e.g., IPI-026 alone or in combination) are administered as a first line of treatment of a medulloblastoma. In other embodiments, the one or more hedgehog inhibitors (e.g., IPI-026 alone or in combination) are administered as a second line of treatment of a medulloblastoma. In yet other embodiments, the one or more hedgehog inhibitors (e.g., IPI-026 alone or in combination) are administered as a third or fourth line of treatment of a medulloblastoma.

In one embodiment, the subject is a patient having a medulloblastoma that has received or is receiving treatment with GDC-0449. In certain embodiments, the subject has become resistant to therapy with GDC-0449.

In yet other embodiments, the resistant tumor or cancer is resistant or refractory to another cancer therapy, such as one or more chemotherapeutic agents. In one embodiment, the hedgehog inhibitor is administered as a single agent or as an adjunct therapy (e.g., in combination with paclitaxel) in platinum resistant cancers or tumors (e.g., platinum resistant ovarian cancer or peritoneal serous cancers).

In yet another aspect, the invention features a treatment regimen and/or a kit that is used to treat, prevent, and/or reduce or inhibit the growth or re-growth of one or more hedgehog-associated cancers or tumors, the metastatic growth, and/or provide the minimal residual disease therapy and/or maintenance therapy, as described herein. The treatment regimen and/or kit includes one or more hedgehog inhibitor, alone or in combination with an therapeutic agent, and, optionally, instructions for use.

Additional embodiments or features of the present invention are as follows:

In some embodiments, the hedgehog inhibitor is a first line treatment for the cancer, i.e., it is used in a subject who has not been previously administered another drug intended to treat the cancer.

In other embodiments, the hedgehog inhibitor is a second line treatment for the cancer, i.e., it is used in a subject who has been previously administered another drug intended to treat the cancer.

In other embodiments, the hedgehog inhibitor is a third or fourth line treatment for the cancer, i.e., it is used in a subject who has been previously administered two or three other drugs intended to treat the cancer.

In some embodiments, a hedgehog inhibitor is administered to a subject following surgical excision/removal of the cancer.

In some embodiments, a hedgehog inhibitor is administered to a subject before, during, and/or after radiation treatment of the cancer.

In one embodiment, the subject treated is a mammal, e.g., a primate, typically a human (e.g., a patient having, or at risk of, a cancer described herein). The subject can be one at risk of having the disorder, e.g., a subject having a relative afflicted with the disorder, or a subject having a genetic trait associated with risk for the disorder. In one embodiment, the subject can be symptomatic or asymptomatic. In one embodiment, the subject is a cancer patient who is undergoing or has undergone cancer therapy (e.g., treatment with a therapeutic agent, radiation therapy and/or surgery. In other embodiments, the subject is a cancer patient in remission (complete or partial remission). In other embodiments, the subject has minimal residual disease, e.g., a cancer patient having one or more residual tumor cells after a primary treatment (e.g., after one or more of chemotherapy, radiotherapy, surgery or targeted therapy). In one embodiment, the subject has, or is identified as having, elevated Gli-1 (e.g., a patient with ovarian cancer that has elevated Gli-1 level or expression).

In other embodiments, the subject is a patient (e.g., a patient with SCLC) who is undergoing or has undergone one or more of radiation, chemotherapy and/or surgery) and shows at least 10%, 20%, 30%, 40%, 50%, 60%, 70%, 80%, 90% or more tumor shrinkage. In one embodiment, the subject is administered one or more hedgehog inhibitors instead of PCI. In other embodiments, the subject is identified, or has, limited stage SCLC. In other embodiments, the subject is identified, or has, extensive SCLC.

In subjects treated with the methods, regimens and/or kits of the invention, treatment can include, but is not limited to, inhibiting or reducing minimal residual disease, inhibiting or reducing tumor growth or re-growth, inhibiting or reducing tumor mass, inhibiting or reducing size or number of metastatic lesions, inhibiting or reducing the development of new metastatic lesions, prolonged survival, prolonged progression-free survival, prolonged time to progression, and/or enhanced quality of life.

In one embodiment, the hedgehog-associated cancer or tumor is a solid tumor, a soft tissue tumor, or a metastatic lesion. Exemplary cancers include, but are not limited to, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphocytic leukemia (ALL), acute myelogenous leukemia (AML), chronic myelogenous leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), multiple myeloma (MM), myelodysplastic syndrome (MDS), myeloproliferative disorder (MPD) (e.g., polycythemia Vera (PV), essential thrombocythemia (ET), agnogenic myeloid metaplasia (AMM) a.k.a. primary myelofibrosis (PMF), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic adenocarcinoma, intraductal papillary mucinous neoplasm (IPMN)), prostate cancer (e.g., prostate adenocarcinoma), skin cancer (e.g., squamous cell carcinoma (SCC), keratoacanthoma (KA), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma, osteosarcoma).

In certain embodiments, the cancer or tumor is selected from bladder cancer, breast cancer, medulloblastoma, colorectal cancer, head and neck cancer, lung cancer (e.g., small cell lung cancer (SCLC), non-small cell lung cancer (NSCLC)), leukemia (e.g., acute lymphocytic leukemia (ALL), acute myelogenous leukemia (AML), chronic myelogenous leukemia (CML), chronic lymphocytic leukemia (CLL)), lymphoma (e.g., Hodgkin lymphoma (HL), non-Hodgkin lymphoma (NHL)), multiple myeloma (MM), osteosarcoma, ovarian cancer, pancreatic cancer, prostate cancer, basal cell carcinoma (BCC)) and chondrosarcoma.

In certain embodiments, the hedgehog-associated cancer or tumor is a ligand-independent or a ligand-dependent cancerous condition. In embodiments where the hedgehog-associated cancer or tumor is a ligand-independent cancerous condition, the cancer or tumor can be associated with a genetic mutation in a component of the hedgehog pathway (e.g., a hedgehog receptor such as Smoothened (Smo) or Patched (Ptc)) that leads to abnormal receptor expression and/or activity. Examples of cancerous conditions involving genetic mutations in a hedgehog receptor that can be treated with the methods of the invention include basal cell carcinoma (BCC) and medulloblastoma. In other embodiments, the hedgehog-associated cancer or tumor is a ligand-dependent cancerous condition, for example, a cancerous condition involving paracrine signaling mechanisms (e.g., between a hedgehog-secreting tumor and the tumor microenvironment, e.g., the surrounding stroma). For example, a hedgehog ligand is secreted from a tumor cell and activates a hedgehog receptor (e.g., Smo and/or Ptc) in the tumor microenvironment (e.g., a nearby stromal cell). Examples of paracrine cancerous conditions that can be treated or prevented with the methods of the invention include desmoplastic tumors, cancers of the pancreas, small cell lung cancer (SCLC), ovary, prostate and bladder. In yet other embodiments, the ligand-dependent cancerous condition can involve direct signaling by a hedgehog ligand to the tumor or cancer cell, e.g., autologous activation of Smo and/or Ptc. Examples of such cancerous conditions include, but are not limited to sarcomas, chondrosarcoma, osteosarcoma, heme malignancies, chronic myelogenous leukemia (CML), SCLC, multiple myeloma (MM), chronic lymphocytic leukemia (CLL), acute lymphoblastic leukemia (ALL), and acute myelogenous leukemia (AML).

In yet other embodiments, the hedgehog-associated cancer or tumor is an advanced and/or metastatic cancer (e.g., a cancer chosen from one or more of: lung cancer (e.g., small cell lung cancer or non-small cell lung cancer), pancreatic cancer, liver cancer, prostate cancer, bladder cancer, ovarian cancer, breast cancer, colon cancer, multiple myeloma, acute myelogenous leukemia (AML), chronic myelogenous leukemia (CML) and neuroendocrine cancer).

In yet another embodiment, the hedgehog-associated cancer or tumor has an alteration in a marker of a hedgehog pathway, including but not limited to, an alteration in a gene or a gene product (e.g., DNA, RNA, protein, including alterations in sequence, activity and/or expression levels) of, a hedgehog ligand (Sonic Hedgehog (SHH), Indian Hedgehog (IHH) or Desert Hedgehog (DHH)), for example, an increase in the levels of a hedgehog ligand polypeptide, detection of a single nucleotide polymorphism of a hedgehog ligand (e.g., a SHH SNP); an alteration in a gene or a gene product (e.g., DNA, RNA, protein, including alterations in sequence, activity and/or expression levels) of, an upstream or downstream component(s) of the hedgehog signaling pathway, e.g., a hedgehog receptor (e.g., patched (PTCH) or smoothened (SMO)), an activator or inhibitor of hedgehog, or a signaling mediator (e.g., Gli1, Gli2, and Gli3). In one embodiment, the hedgehog-associated cancer or tumor has an alteration in the marker of the hedgehog pathway resulting from exposure to another cancer therapy, such as one or more therapeutic agents, radiation therapy and/or surgery. In one embodiment, the hedgehog-associated cancer or tumor has an elevated expression of a hedgehog ligand, e.g., Sonic Hedgehog (SHH). Exemplary hedgehog-associated cancers or tumors having elevated expression of SHH, include but are not limited to, pancreatic ductal carcinomas, colon adenocarcinoma, ovarian cystadenocarcinoma and prostate adenocarcinoma. Another hedgehog-associated cancer that can be treated with the methods and compositions of the invention is chondrosarcoma. In certain embodiment, an increased level (e.g., expression level) of a hedgehog marker is associated with decreased survival. For example, elevated expression of Gli-1 in stroma is associated with decreased survival of a patient with ovarian cancer.

In one embodiment, the hedgehog inhibitor reduces or inhibits the activity of a hedgehog receptor, e.g., Smoothened and/or Patched. Thus, the hedgehog inhibitor can be a Smoothened inhibitor and/or a Patched inhibitor. In some embodiments, the hedgehog inhibitor reduces or blocks Smoothened activity (e.g., signaling), in a tumor microenvironment, thereby causing one or more of: (i) depleting or reducing desmoplastic stroma; (ii) increasing the vascularity of the tumor; or (iii) rendering the tumor more accessible to chemotherapy.

In another embodiment, the hedgehog inhibitor targets a ligand-dependent cancer or tumor, e.g., the inhibitor targets one or more of the tumor microenvironment, a tumor cell or other residual diseases. In some embodiments, hedgehog inhibitor targets the tumor microenvironment of a ligand-dependent cancer (e.g., a desmoplastic tumors, such as pancreatic cancer and/or neuroendocrine tumors). In such embodiments, the hedgehog inhibitor can decrease fibrosis, thus leading to improved drug delivery and/or survival.

In other embodiments, the hedgehog inhibitor targets a ligand-independent cancer or tumor.

In yet other embodiments, the hedgehog inhibitor is a hedgehog receptor inhibitor, e.g., a Smoothened inhibitor and/or a Patched inhibitor.

In one embodiment, the hedgehog inhibitor used in the methods or compositions described herein is a compound as follows:

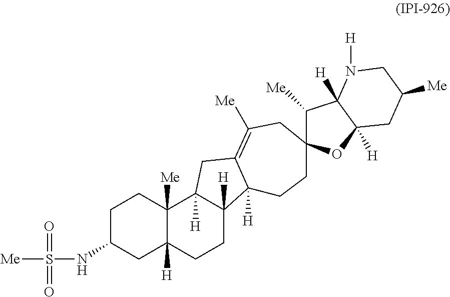

or a pharmaceutically acceptable salt thereof. This compound, or a pharmaceutically acceptable salt thereof, is also referred to herein as IPI-926. An example of a pharmaceutically acceptable salt of the compound of formula I is the hydrochloride salt.

In some embodiments, the hedgehog inhibitor is administered as a pharmaceutical composition comprising the hedgehog inhibitor, or a pharmaceutically acceptable salt thereof, and a pharmaceutically acceptable excipient. In one embodiment, one or more different hedgehog inhibitors are administered in combination.

In certain embodiments, one or more hedgehog inhibitors are administered, or are present in the composition, e.g., the pharmaceutical composition.

The hedgehog inhibitors described herein can be administered to the subject systemically (e.g., orally, parenterally, subcutaneously, intravenously, rectally, intramuscularly, intraperitoneally, intranasally, transdermally, or by inhalation or intracavitary installation). Typically, the hedgehog inhibitors are administered orally.

In one embodiment, the hedgehog inhibitor is IPI-926. IPI-926 can be administered orally in a daily schedule at a dose of about 20 mg to 200 mg, typically about 50 to 150 mg, 75 to 140 mg, and more typically 120 to 130 mg, alone or in combination with a second agent as described herein.

The methods and compositions of the invention can optionally be used in combination with one or more other cancer therapies (e.g., one or more therapeutic agents surgery and/or radiation). In one embodiment, the methods and compositions of the invention are used in combination with surgical and/or radiation procedures. In other embodiments, the methods and compositions of the invention are used in combination with one or more therapeutic agents.

In one embodiment, the hedgehog-associated cancer or tumor treated is a lung cancer (e.g., small cell lung cancer or non-small cell lung cancer); the hedgehog inhibitor is administered concurrently or following cessation of chemotherapy (e.g., etoposide/carboplatin combination, or tyrosine kinase inhibition (e.g., Geftinimib)); the tumor recurrence is delayed by at least 5, 10, 15, 20, 25 or more days; the size of the tumor re-growth is reduced by at least 10%, 20%, 30%, 40%, 50%, 60%, 65%, 70%, 80%, or at least 90%, compared to an untreated subject, or as shown in FIGS. 1 and 9.

In another embodiment, the hedgehog-associated cancer or tumor treated is an ovarian cancer; the hedgehog inhibitor is administered concurrently or without a substantial delay after cessation of cancer therapy (e.g., simultaneously with, or less than 15, 10, 8, 6, 5, 4, 3 days, or less than 48, 36, 24, 14, 10, 9, 8, 7, 6, 5, 4, 3, 2, or 1 hour after cessation of chemotherapy (e.g., carboplatin/taxol combination)); the tumor recurrence is delayed by at least 5, 10, 15, 20, 25 or more days; the size of the tumor re-growth is reduced by at least 10%, 20%, 30%, 40%, 50%, 60%, 65%, 70%, 80%, or at least 90%, compared to an untreated subject, or as shown in FIG. 6.

In yet another embodiment, the hedgehog-associated cancer or tumor treated is an prostate cancer; the hedgehog inhibitor is administered concurrently or without a substantial delay after cessation of cancer therapy (e.g., simultaneously with, or less than 15, 10, 8, 6, 5, 4, 3 days, or less than 48, 36, 24, 14, 10, 9, 8, 7, 6, 5, 4, 3, 2, or 1 hour after cessation of chemotherapy (e.g., docetaxel)); the tumor recurrence is delayed by at least 5, 10, 15, 20, 25 or more days; the size of the tumor re-growth is reduced by at least 10%, 20%, 30%, 40%, 50%, 60%, 65%, 70%, 80%, or at least 90%, compared to an untreated subject, or as shown in FIG. 7.

In some embodiments, the hedgehog inhibitor is administered to a subject, e.g., a cancer patient who is undergoing or has undergone cancer therapy (e.g., treatment with a therapeutic agent, radiation therapy and/or surgery). In other embodiments, the hedgehog inhibitor is administered concurrently with the cancer therapy (e.g., having at least some period of overlap between administration of the therapeutic agent, radiation therapy and/or surgery and the administration of the hedgehog inhibitor; for example, at least 1, 2, 3, 4, 5, 10, 15, 24, 36, or 48 hours; at least 1, 2, 3, 4, 5, 6, 7, 10, 14, or 20 days; at least 1, 2, 3, 4, 5, 6, 8, 10, or 12 months; prior to cessation of cancer therapy as described herein). In instances of concurrent administration, the hedgehog inhibitor can continue to be administered after the cancer therapy has ceased. In other embodiments, the hedgehog inhibitor is administered after cancer therapy has ceased (i.e., with no period of overlap with the administration of the therapeutic agent, radiation therapy and/or surgery), e.g., as a maintenance therapy as described herein.

In other embodiments, the hedgehog inhibitor is administered to a subject, e.g., a cancer patient who is undergoing or has undergone one or more of radiation, chemotherapy and/or surgery) and shows at least 10%, 20%, 30%, 40%, 50%, 60%, 70%, 80%, 90% or more tumor shrinkage. In other embodiments, the hedgehog inhibitor is administered concurrently with the cancer therapy (e.g., having at least some period of overlap between administration of the therapeutic agent, radiation therapy and/or surgery and the administration of the hedgehog inhibitor; for example, at least 1, 2, 3, 4, 5, 10, 15, 24, 36, or 48 hours; at least 1, 2, 3, 4, 5, 6, 7, 10, 14, or 20 days; at least 1, 2, 3, 4, 5, 6, 8, 10, or 12 months; prior to cessation of cancer therapy as described herein). In instances of concurrent administration, the hedgehog inhibitor can continue to be administered after the cancer therapy has ceased. In other embodiments, the hedgehog inhibitor is administered after cancer therapy has ceased (i.e., with no period of overlap with the administration of the therapeutic agent, radiation therapy and/or surgery), e.g., as a maintenance therapy as described herein.

Any combination of the hedgehog inhibitor and other cancer therapies (e.g., one or more therapeutic agents, surgery and/or radiation) can be used. For example, the hedgehog inhibitor and other cancer therapies can be administered during periods of active disorder, or during a period of remission or less active disease. The hedgehog inhibitor and other cancer therapies can be administered before treatment, concurrently with treatment, post-treatment, or during remission of the disorder. In one embodiment, the cancer therapy is administered simultaneously or sequentially with the hedgehog inhibitor.

In one embodiment, hedgehog inhibitor is administered in combination with one or more of an anti-cancer agent (e.g., a cytotoxic or a cytostatic agent), surgery or radiation. In one embodiment, the anti-cancer agent is chosen from a tyrosine kinase inhibitor, a taxane, gemcitabine, cisplatin, epirubicin, 5-fluorouracil, a VEGF inhibitor, leucovorin, oxaplatin, Ara-c, or a combination thereof. In other embodiments, the anti-cancer agent is chosen from one or more of an insulin-like growth factor receptor (IGF-1R) inhibitor, a PI3K inhibitor, an HSP90 inhibitor, folfirinox, a BRAF inhibitor, a MEK inhibitor, or a JAK2 inhibitor. Exemplary tyrosine kinase inhibitors include, but are not limited to, sunitinib, erlotinib, gefitinib, sorafenib, icotinib, lapatinib, neratinib, vandetanib, BIBW 2992 or XL-647. Other tyrosine kinase inhibitor can be chosen from a monoclonal antibody against EGFR, e.g., cetuximab, panitumumab, zalutumumab, nimotuzumab necitumumab or matuzumab. Additional exemplary combination therapies are described herein.

In one embodiment, the hedgehog inhibitor (e.g., IPI-926) is administered in combination with a PI3K inhibitor. In one embodiment, the PI3K inhibitor is an inhibitor of delta and gamma isoforms of PI3K. Exemplary PI3K inhibitors that can be used in combination are described in, e.g., WO 09/088,990; WO 09/088,086; WO 2011/008302; WO 2010/036380; WO 2010/006086, WO 09/114,870, WO 05/113556; US 2009/0312310, US 2011/0046165. Additional PI3K inhibitors that can be used in combination with the hedgehog inhibitors, include but are not limited to, GSK 2126458, GDC-0980, GDC-0941, Sanofi XL147, XL756, XL147, PF-46915032, Novartis BEZ 235, BKM 120, CAL-101, CAL 263, SF1126 and PX-886. In one embodiment, the PI3K inhibitor is an isoquinolinone. In one embodiment, the PI3K inhibitor is INK1197 or a derivative thereof. In other embodiments, the PI3K inhibitor is INK1117 or a derivative thereof. The hedgehog inhibitor and the PI3K inhibitor can be administered simultaneously or sequentially as described herein. In certain embodiments, the inhibitors are administered in the same composition, or in different compositions, as described hereinbelow.

In other embodiments, the hedgehog inhibitor and the therapeutic agent are administered as separate compositions, e.g., pharmaceutical compositions. In other embodiments, the hedgehog inhibitor and the therapeutic agent are administered separately, but via the same route (e.g., both orally or both intravenously). In still other instances, the hedgehog inhibitor and the therapeutic agent are administered in the same composition, e.g., pharmaceutical composition.

In one embodiment, the hedgehog inhibitor is administered prior to detection of a metastatic lesion.

The methods of the invention can further include the step of monitoring the subject, e.g., for a change (e.g., an increase or decrease) in one or more of: tumor size; hedgehog levels or signaling; stromal activation; levels of one or more cancer markers; the rate of appearance of new lesions, e.g., in a bone scan; the appearance of new disease-related symptoms; the size of soft tissue mass, e.g., a decreased or stabilization; quality of life, e.g., amount of disease associated pain, e.g., bone pain; or any other parameter related to clinical outcome. The subject can be monitored in one or more of the following periods: prior to beginning of treatment; during the treatment; or after one or more elements of the treatment have been administered. Monitoring can be used to evaluate the need for further treatment with the same hedgehog inhibitor, alone or in combination with, the same therapeutic agent, or for additional treatment with additional agents. Generally, a decrease in one or more of the parameters described above is indicative of the improved condition of the subject, although with serum hemoglobin levels, an increase can be associated with the improved condition of the subject.

The methods of the invention can further include the step of analyzing a nucleic acid or protein from the subject, e.g., analyzing the genotype of the subject. In one embodiment, a hedgehog protein, or a nucleic acid encoding a hedgehog ligand and/or an upstream or downstream component(s) of the hedgehog signaling, e.g., a receptor, activator or inhibitor of hedgehog, is analyzed. The elevated hedgehog ligand can be detected in blood, urine, circulating tumor cells, a tumor biopsy or a bone marrow biopsy. The elevated hedgehog ligand can also be detected by systemic administration of a labeled form of an antibody to a hedgehog ligand followed by imaging. The analysis can be used, e.g., to evaluate the suitability of, or to choose between alternative treatments, e.g., a particular dosage, mode of delivery, time of delivery, inclusion of adjunctive therapy, e.g., administration in combination with a second agent, or generally to determine the subject's probable drug response phenotype or genotype. The nucleic acid or protein can be analyzed at any stage of treatment, but preferably, prior to administration of the hedgehog inhibitor and/or therapeutic agent, to thereby determine appropriate dosage(s) and treatment regimen(s) of the hedgehog inhibitor (e.g., amount per treatment or frequency of treatments) for prophylactic or therapeutic treatment of the subject.

In one embodiment, an alteration in a marker of a hedgehog pathway is analyzed, including but not limited to, an alteration in a gene or a gene product (e.g., DNA, RNA, protein, including alterations in sequence, activity and/or expression levels) of, a hedgehog ligand (Sonic Hedgehog (SHH), Indian Hedgehog (IHH) or Desert Hedgehog (DHH)), for example, an increase in the levels of a hedgehog ligand polypeptide, detection of a single nucleotide polymorphism of a hedgehog ligand (e.g., a SHH SNP); an alteration in a gene or a gene product (e.g., DNA, RNA, protein, including alterations in sequence, activity and/or expression levels) of, an upstream or downstream component(s) of the hedgehog signaling pathway, e.g., a hedgehog receptor (e.g., patched (PTCH) or smoothened (SMO)), an activator or inhibitor of hedgehog, or a signaling mediator (e.g., Gli1, Gli2, and Gli3). In one embodiment, the alteration in the marker of the hedgehog pathway results from exposure to another cancer therapy, such as one or more therapeutic agents, radiation therapy and/or surgery. In one embodiment, the hedgehog-associated cancer or tumor has an elevated expression of a hedgehog ligand, e.g., Sonic Hedgehog (SHH). Exemplary hedgehog-associated cancers or tumors having elevated expression of SHH, include but are not limited to, pancreatic ductal carcinomas, colon adenocarcinoma, ovarian cystadenocarcinoma and prostate adenocarcinoma. Another hedgehog-associated cancer that can be treated with the methods and compositions of the invention is chondrosarcoma. In certain embodiment, an increased level (e.g., expression level) of a hedgehog marker is associated with decreased survival. For example, elevated expression of Gli-1 in stroma is associated with decreased survival of a patient with ovarian cancer.

In certain embodiments, the methods of the invention further include the step of detecting elevated hedgehog ligand in the subject, prior to, or after, administering a hedgehog inhibitor to the patient. The elevated hedgehog ligand can be detected in blood, urine, circulating tumor cells, a tumor biopsy or a bone marrow biopsy. The elevated hedgehog ligand can also be detected by systemic administration of a labeled form of an antibody to a hedgehog ligand followed by imaging. The step of detecting elevated hedgehog ligand can include the steps of measuring hedgehog ligand in the patient prior to administration of the other cancer therapy, measuring hedgehog ligand in the patient after administration of the other cancer therapy, and determining if the amount of hedgehog ligand after administration of the other chemotherapy is greater than the amount of hedgehog ligand before administration of the other chemotherapy. The other cancer therapy can be, for example, a therapeutic agent or radiation therapy.

In another aspect, the method further includes the step of identifying one or more therapeutic agents that elevate hedgehog ligand expression in a tumor (e.g., a neuroendocrine cancer), and administering a therapeutically effective amount of the one or more therapeutic agents that elevate hedgehog ligand expression in the tumor and a therapeutically effective amount of a hedgehog inhibitor. The step of identifying the therapeutic agent that elevate hedgehog expression can include the steps of exposing cells from the tumor to one or more therapeutic agents in vitro and measuring hedgehog ligand in the cells.

In another aspect, the invention features a composition, e.g., a pharmaceutical composition that includes one or more hedgehog inhibitors, e.g., a hedgehog inhibitor as described herein, and one or more therapeutic agents. The composition can further include a pharmaceutically-acceptable carrier or excipient.

In another aspect, the invention features the use of a hedgehog inhibitor, alone or in combination with one or more cancer therapies (e.g., one or more therapeutic agents, radiation and/or surgery), for the treatment of cancers or tumors.

All publications, patent applications, patents, and other references mentioned herein are incorporated by reference in their entirety.

Other features, objects, and advantages of the invention will be apparent from the description and drawings, and from the claims.

### DEFINITIONS

Definitions of specific functional groups and chemical terms are described in detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in, for example, Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Certain compounds of the present invention can comprise one or more asymmetric centers, and thus can exist in various isomeric forms, i.e., stereoisomers (enantiomers, diastereomers, cis-trans isomers, E/Z isomers, etc.). Thus, inventive compounds and pharmaceutical compositions thereof can be in the form of an individual enantiomer, diastereomer or other geometric isomer, or can be in the form of a mixture of stereoisomers. Enantiomers, diastereomers and other geometric isomers can be isolated from mixtures (including racemic mixtures) by any method known to those skilled in the art, including chiral high pressure liquid chromatography (HPLC) and the formation and crystallization of chiral salts or prepared by asymmetric syntheses; see, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

Carbon atoms, unless otherwise specified, can optionally be substituted with one or more substituents. The number of substituents is typically limited by the number of available valences on the carbon atom, and can be substituted by replacement of one or more of the hydrogen atoms that would be available on the unsubstituted group. Suitable substituents are known in the art and include, but are not limited to, alkyl, alkenyl, alkynyl, alkoxy, alkoxy, aryl, aryloxy, arylthio, aralkyl, heteroaryl, heteroaralkyl, cycloalkyl, heterocyclyl, halo, azido, hydroxyl, thio, alkthiooxy, amino, nitro, nitrile, imino, amido, carboxylic acid, aldehyde, carbonyl, ester, silyl, alkylthio, haloalkyl (e.g., perfluoroalkyl such as —CF₃), ═O, ═S, and the like.

When a range of values is listed, it is intended to encompass each value and sub-range within the range. For example, an alkyl group containing 1-6 carbon atoms (C₁₋₆ alkyl) is intended to encompass, C₁, C₂, C₃, C₄, C₅, C₆, C₁₋₆, C₂₋₆, C₃₋₆, C₄₋₆, C₅₋₆, C₁₋₅, C₂₋₅, C₃₋₅, C₄₋₅, C₁₋₄, C₂₋₄, C₃₋₄, C₁₋₃, C₂₋₃, and C₁₋₂ alkyl.

The term “alkyl,” as used herein, refers to saturated, straight- or branched-chain hydrocarbon radical containing between one and thirty carbon atoms. In certain embodiments, the alkyl group contains 1-20 carbon atoms. Alkyl groups, unless otherwise specified, can optionally be substituted with one or more substituents. In certain embodiments, the alkyl group contains 1-10 carbon atoms. In certain embodiments, the alkyl group contains 1-6 carbon atoms. In certain embodiments, the alkyl group contains 1-5 carbon atoms. In certain embodiments, the alkyl group contains 1-4 carbon atoms. In certain embodiments, the alkyl group contains 1-3 carbon atoms. In certain embodiments, the alkyl group contains 1-2 carbon atoms. In certain embodiments, the alkyl group contains 1 carbon atom. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl,” as used herein, denotes a straight- or branched-chain hydrocarbon radical having at least one carbon-carbon double bond by the removal of a single hydrogen atom, and containing between two and thirty carbon atoms. Alkenyl groups, unless otherwise specified, can optionally be substituted with one or more substituents. In certain embodiments, the alkenyl group contains 2-20 carbon atoms. In certain embodiments, the alkenyl group contains 2-10 carbon atoms. In certain embodiments, the alkenyl group contains 2-6 carbon atoms. In certain embodiments, the alkenyl group contains 2-5 carbon atoms. In certain embodiments, the alkenyl group contains 2-4 carbon atoms. In certain embodiment, the alkenyl group contains 2-3 carbon atoms. In certain embodiments, the alkenyl group contains 2 carbon atoms. Alkenyl groups include, for example, ethenyl, propenyl, butenyl, 1-methyl-2-buten-1-yl, and the like.

The term “alkynyl,” as used herein, denotes a straight- or branched-chain hydrocarbon radical having at least one carbon-carbon triple bond by the removal of a single hydrogen atom, and containing between two and thirty carbon atoms. Alkynyl groups, unless otherwise specified, can optionally be substituted with one or more substituents. In certain embodiments, the alkynyl group contains 2-20 carbon atoms. In certain embodiments, the alkynyl group contains 2-10 carbon atoms. In certain embodiments, the alkynyl group contains 2-6 carbon atoms. In certain embodiments, the alkynyl group contains 2-5 carbon atoms. In certain embodiments, the alkynyl group contains 2-4 carbon atoms. In certain embodiments, the alkynyl group contains 2-3 carbon atoms. In certain embodiments, the alkynyl group contains 2 carbon atoms. Representative alkynyl groups include, but are not limited to, ethynyl, 2-propynyl (propargyl), 1-propynyl, and the like.

The terms “cycloalkyl”, used alone or as part of a larger moiety, refer to a saturated monocyclic or bicyclic hydrocarbon ring system having from 3-15 carbon ring members. Cycloalkyl groups, unless otherwise specified, can optionally be substituted with one or more substituents. In certain embodiments, cycloalkyl groups contain 3-10 carbon ring members. In certain embodiments, cycloalkyl groups contain 3-9 carbon ring members. In certain embodiments, cycloalkyl groups contain 3-8 carbon ring members. In certain embodiments, cycloalkyl groups contain 3-7 carbon ring members. In certain embodiments, cycloalkyl groups contain 3-6 carbon ring members. In certain embodiments, cycloalkyl groups contain 3-5 carbon ring members. Cycloalkyl groups include, without limitation, cyclopropyl, cyclobutyl, cyclopentyl, cyclohexyl, cycloheptyl, and cyclooctyl. The term “cycloalkyl” also includes saturated hydrocarbon ring systems that are fused to one or more aryl or heteroaryl rings, such as decahydronaphthyl or tetrahydronaphthyl, where the point of attachment is on the saturated hydrocarbon ring.

The term “aryl” used alone or as part of a larger moiety (as in “aralkyl”), refers to an aromatic monocyclic and bicyclic hydrocarbon ring system having a total of 6-10 carbon ring members. Aryl groups, unless otherwise specified, can optionally be substituted with one or more substituents. In certain embodiments of the present invention, “aryl” refers to an aromatic ring system which includes, but not limited to, phenyl, biphenyl, naphthyl, anthrancyl and the like, which can bear one or more substituents. Also included within the scope of the term “aryl”, as it is used herein, is a group in which an aryl ring is fused to one or more non-aromatic rings, such as indanyl, phthalimidyl or tetrahydronaphthalyl, and the like, where the point of attachment is on the aryl ring.

The term “aralkyl” refers to an alkyl group, as defined herein, substituted by aryl group, as defined herein, wherein the point of attachment is on the alkyl group.

The term “heteroatom” refers to boron, phosphorus, selenium, nitrogen, oxygen, or sulfur, and includes any oxidized form of nitrogen or sulfur, and any quaternized form of abasic nitrogen.

The terms “heteroaryl” used alone or as part of a larger moiety, e.g., “heteroaralkyl”, refer to an aromatic monocyclic or bicyclic hydrocarbon ring system having 5-10 ring atoms wherein the ring atoms comprise, in addition to carbon atoms, from one to five heteroatoms. Heteroaryl groups, unless otherwise specified, can optionally be substituted with one or more substituents. When used in reference to a ring atom of a heteroaryl group, the term “nitrogen” includes a substituted nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaryl ring is fused to one or more aryl, cycloalkyl or heterocycloalkyl rings, wherein the point of attachment is on the heteroaryl ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl.

The term “heteroaralkyl” refers to an alkyl group, as defined herein, substituted by a heteroaryl group, as defined herein, wherein the point of attachment is on the alkyl group.

As used herein, the terms “heterocycloalkyl” or “heterocyclyl” refer to a stable non-aromatic 5-7 membered monocyclic hydrocarbon or stable non-aromatic 7-10 membered bicyclic hydrocarbon that is either saturated or partially unsaturated, and having, in addition to carbon atoms, one or more heteroatoms. Heterocycloalkyl or heterocyclyl groups, unless otherwise specified, can optionally be substituted with one or more substituents. When used in reference to a ring atom of a heterocycloalkyl group, the term “nitrogen” includes a substituted nitrogen. The point of attachment of a heterocycloalkyl group can be at any of its heteroatom or carbon ring atoms that results in a stable structure. Examples of heterocycloalkyl groups include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. “Heterocycloalkyl” also include groups in which the heterocycloalkyl ring is fused to one or more aryl, heteroaryl or cycloalkyl rings, such as indolinyl, chromanyl, phenanthridinyl, or tetrahydroquinolinyl, where the radical or point of attachment is on the heterocycloalkyl ring.

The term “unsaturated”, as used herein, means that a moiety has one or more double or triple bonds.

As used herein, the term “partially unsaturated” refers to a ring moiety that includes at least one double or triple bond. The term “partially unsaturated” is intended to encompass rings having multiple sites of unsaturation, but is not intended to include aromatic groups, such as aryl or heteroaryl moieties, as defined herein.

The term “diradical” as used herein refers to an alkyl, alkenyl, alkynyl, aryl, cycloalkyl, heterocycloalkyl, aralkyl, heteroaryl, and heteroaralkyl groups, as described herein, wherein 2 hydrogen atoms are removed to form a divalent moiety (e.g., an alkyl diradical, an alkenyl diradical, an alkynyl diradical, an aryl diradical, a cycloalkyl diradical, a heterocycloalkyl diradical, an aralkyl diradical, a heteroaryl diradical, and a heteroaralkyl diradical). Diradicals are typically end with a suffix of “-ene”. For example, alkyl diradicals are referred to as alkylenes (for example:

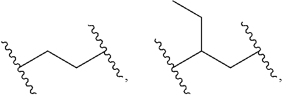

and —(CR′₂)ₓ— wherein R′ is hydrogen or other substituent and x is 1, 2, 3, 4, 5 or 6); alkenyl diradicals are referred to as “alkenylenes”; alkynyl diradicals are referred to as “alkynylenes”; aryl and aralkyl diradicals are referred to as “arylenes” and “aralkylenes”, respectively (for example:

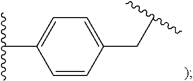

heteroaryl and heteroaralkyl diradicals are referred to as “heteroarylenes” and “heteroaralkylenes”, respectively (for example:

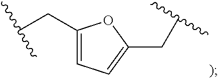

cycloalkyl diradicals are referred to as “cycloalkylenes”; heterocycloalkyl diradicals are referred to as “heterocycloalkylenes”; and the like.

The terms “halo”, “halogen” and “halide” as used herein refer to an atom selected from fluorine (fluoro, F), chlorine (chloro, Cl), bromine (bromo, Br), and iodine (iodo, I).

As used herein, the term “haloalkyl” refers to an alkyl group, as described herein, wherein one or more of the hydrogen atoms of the alkyl group is replaced with one or more halogen atoms. In certain embodiments, the haloalkyl group is a perhaloalkyl group, that is, having all of the hydrogen atoms of the alkyl group replaced with halogens (e.g., such as the perfluoroalkyl group —CF₃).

As used herein, the term “azido” refers to the group —N₃.

As used herein, the term “nitrile” refers to the group —CN.

As used herein, the term “nitro” refers to the group —NO₂.

As used herein, the term “hydroxyl” or “hydroxy” refers to the group —OH.

As used herein, the term “thiol” or “thio” refers to the group —SH.

As used herein, the term “carboxylic acid” refers to the group —CO₂H.

As used herein, the term “aldehyde” refers to the group —CHO.

As used herein, the term “alkoxy” refers to the group —OR′, wherein R′ is an alkyl, alkenyl or alkynyl group, as defined herein.

As used herein, the term “aryloxy” refers to the group —OR′, wherein each R′ is an aryl or heteroaryl group, as defined herein.

As used herein, the term “alkthiooxy” refers to the group —SR′, wherein each R′ is, independently, a carbon moiety, such as, for example, an alkyl, alkenyl, or alkynyl group, as defined herein.

As used herein, the term “arylthio” refers to the group —SR′, wherein each R′ is an aryl or heteroaryl group, as defined herein.

As used herein, the term “amino” refers to the group —NR′₂, wherein each R′ is, independently, hydrogen, a carbon moiety, such as, for example, an alkyl, alkenyl, alkynyl, aryl or heteroaryl group, as defined herein, or two R′ groups together with the nitrogen atom to which they are bound form a 5-8 membered ring.

As used herein, the term “carbonyl” refers to the group —C(═O)R′, wherein R′ is, independently, a carbon moiety, such as, for example, an alkyl, alkenyl, alkynyl, aryl or heteroaryl group, as defined herein.

As used herein, the term “ester” refers to the group —C(═O)OR′ or —OC(═O)R′ wherein each R′ is, independently, a carbon moiety, such as, for example, an alkyl, alkenyl, alkynyl, aryl or heteroaryl group, as defined herein.

As used herein, the term “amide” or “amido” refers to the group —C(═O)N(R′)₂ or —NR′C(═O)R′ wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

The term “sulfonamido” or “sulfonamide” refers to the group —N(R′)SO₂R′ or —SO₂N(R′)₂, wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

The term “sulfamido” or “sulfamide” refers to the group —NR′SO₂N(R′)₂, wherein each R′ is, independently, hydrogen or a carbon moiety, such as, for example, an alkyl, alkenyl, alkynyl, aryl or heteroaryl group, as defined herein, or two R′ groups together with the nitrogen atom to which they are bound form a 5-8 membered ring.

As used herein, the term “imide” or “imido” refers to the group —C(═NR′)N(R′)₂ or —NR′C(═NR′)R′ wherein each R′ is, independently, hydrogen or a carbon moiety, such as, for example, an alkyl, alkenyl, alkynyl, aryl or heteroaryl group, as defined herein, or wherein two R′ groups together with the nitrogen atom to which they are bound form a 5-8 membered ring.

As used herein “silyl” refers to the group —SiR′ wherein R′ is a carbon moiety, such as, for example, an alkyl, alkenyl, alkynyl, aryl or heteroaryl group.

In some cases, the hedgehog inhibitor can contain one or more basic functional groups (e.g., such as an amino group), and thus is capable of forming pharmaceutically acceptable salts with pharmaceutically acceptable acids. The term “pharmaceutically acceptable salts” in these instances refers to the relatively non-toxic, inorganic and organic acid addition salts. These salts can be prepared in situ in the administration vehicle or the dosage form manufacturing process, or by separately treating the compound in its free base form with a suitable acid. Examples of pharmaceutically acceptable, nontoxic acid addition salts from inorganic acids include, but are not limited to, hydrochloric, hydrobromic, phosphoric, sulfuric, nitric and perchloric acid or from organic acids include, but are not limited to, acetic, adipic, alginic, ascorbic, aspartic, 2-acetoxybenzoic, benzenesulfonic, benzoic, bisulfonic, boric, butyric, camphoric, camphorsulfonic, citric, cyclopentanepropionic, digluconic, dodecylsulfonic, ethanesulfonic, 1,2-ethanedisulfonic, formic, fumaric, glucoheptonic, glycerophosphonic, gluconic, hemisulfonic, heptanoic, hexanoic, hydroiodic, 2-hydroxyethanesulfonic, hydroxymaleic, isothionic, lactobionic, lactic, lauric, lauryl sulfonic, malic, maleic, malonic, methanesulfonic, 2-naphthalenesulfonic, napthylic, nicotinic, oleic, oxalic, palmitic, pamoic, pectinic, persulfonic, 3-phenylpropionic, picric, pivalic, propionic, phenylacetic, stearic, succinic, salicyclic, sulfanilic, tartaric, thiocyanic, p-toluenesulfonic, undecanoic, and valeric acid addition salts, and the like. In other cases, the hedgehog inhibitor can contain one or more acidic functional groups, and thus is capable of forming pharmaceutically acceptable salts with pharmaceutically acceptable bases. The term “pharmaceutically acceptable salts” in these instances refers to the relatively non-toxic, inorganic and organic base addition salts. These salts can likewise be prepared in situ in the administration vehicle or the dosage form manufacturing process, or by separately treating the compound in its free acid form with a suitable base. Examples of suitable bases include, but are not limited to, metal hydroxides, metal carbonates or metal bicarbonates, wherein the metal is an alkali or alkaline earth metal such as lithium, sodium, potassium, calcium, magnesium, or aluminum. Suitable bases can also include ammonia or organic primary, secondary or tertiary amines. Representative organic amines useful for the formation of base addition salts include, for example, ethylamine, diethylamine, ethylenediamine, ethanolamine, diethanolamine, piperazine and the like (see, e.g., Berge et al., supra).

The term “solvate” refers to a compound of the present invention having either a stoichiometric or non-stoichiometric amount of a solvent associated with the compound. The solvent can be water (i.e., a hydrate), and each molecule of inhibitor can be associated with one or more molecules of water (e.g., monohydrate, dihydrate, trihydrate, etc.). The solvent can also be an alcohol (e.g., methanol, ethanol, propanol, isopropanol, etc.), a glycol (e.g., propylene glycol), an ether (e.g., diethyl ether), an ester (e.g., ethyl acetate), or any other suitable solvent. The hedgehog inhibitor can also exist as a mixed solvate (i.e., associated with two or more different solvents).

The term “sugar” as used herein refers to a natural or an unnatural monosaccharide, disaccharide or oligosaccharide comprising one or more pyranose or furanose rings. The sugar can be covalently bonded to the steroidal alkaloid of the present invention through an ether linkage or through an alkyl linkage. In certain embodiments the saccharide moiety can be covalently bonded to a steroidal alkaloid of the present invention at an anomeric center of a saccharide ring. Sugars can include, but are not limited to ribose, arabinose, xylose, lyxose, allose, altrose, glucose, mannose, gulose, idose, galactose, talose, glucose, and trehalose.

For convenience, certain terms are defined herein.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, and unless otherwise specified, the terms “treat,” “treating” and “treatment” contemplate an action that occurs while a patient is suffering from cancer, which reduces the severity of the cancer, or retards or slows the progression of the cancer.

As used herein, unless otherwise specified, the terms “prevent,” “preventing” and “prevention” contemplate an action that occurs before a patient begins to suffer from the regrowth of the cancer and/or which inhibits or reduces the severity of the cancer.

As used herein, and unless otherwise specified, the terms “manage,” “managing” and “management” encompass preventing the recurrence of the cancer in a patient who has already suffered from the cancer, and/or lengthening the time that a patient who has suffered from the cancer remains in remission. The terms encompass modulating the threshold, development and/or duration of the cancer, or changing the way that a patient responds to the cancer.

As used therein, the term “maintenance therapy” refers to an extended therapy, usually administered at a diminished dose that follows another treatment regimen. For example, administration of a hedgehog inhibitor(s) that follows one or more other forms of chemotherapy. In one embodiment, the maintenance therapy is administered to a subject who has one or more cancers in remission to reduce, delay or prevent a relapse or recurrence of the cancer(s) in the subject, and/or lengthening the time that the subject who has suffered from the cancer(s) remains in remission. Complete remission is not necessary for initiating maintenance therapy, as the maintenance therapy can be administered to a subject when a complete cure or remission is not attainable.

As used herein, and unless otherwise specified, a “therapeutically effective amount” of a compound is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent.

As used herein, and unless otherwise specified, a “prophylactically effective amount” of a compound is an amount sufficient to prevent regrowth of the cancer, or one or more symptoms associated with the cancer, or prevent its recurrence. A prophylactically effective amount of a compound means an amount of the compound, alone or in combination with other therapeutic agents, which provides a prophylactic benefit in the prevention of the cancer. The term “prophylactically effective amount” can encompass an amount that improves overall prophylaxis or enhances the prophylactic efficacy of another prophylactic agent.

As used herein, “cancer” and “tumor” are synonymous terms. The term “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. Cancer cells also include cancer stem cells (CSC). As used herein, the term “cancer” includes premalignant as well as malignant cancers.

As used herein, “cancer therapy” and “cancer treatment” are synonymous terms.

As used herein “therapeutic agent” and “drug” are synonymous terms and are meant to include both biotherapeutic agents (e.g., cancer biologics) as well as chemotherapeutic agents.

The term “subject” as used herein, refers to an animal, typically a human (i.e., a male or female of any age group, e.g., a pediatric subject (e.g., infant, child, adolescent) or adult subject (e.g., young adult, middle-aged adult or senior adult) or other mammal, such as primates (e.g., cynomolgus monkeys, rhesus monkeys); commercially relevant mammals such as cattle, pigs, horses, sheep, goats, cats, and/or dogs; and/or birds, including commercially relevant birds such as chickens, ducks, geese, and/or turkeys, that will be or has been the object of treatment, observation, and/or experiment. When the term is used in conjunction with administration of a compound or drug, then the subject has been the object of treatment, observation, and/or administration of the compound or drug.

Additional terms are defined throughout the specification.

### DESCRIPTION OF THE FIGURES

FIG. 1 shows data indicating that IPI-926 is efficacious post-chemotherapy in a primary small cell lung cancer (SCLC) model of minimal residual disease. FIG. 1 is a series of line graphs showing the effect in tumor size (mm³) as a function of days of treatment of mice having an LX22 primary small cell lung tumor with IPI-926 alone (“IPI-926”), etoposide/carboplatin followed by vehicle control (“E/P→Vehicle”), E/P followed by IPI-926 (“E/P→IPI-926”) and vehicle control. Mice were treated for 5 weeks total with IPI-926 follow-up treatment at 40 mg/kg PO QD.

FIG. 2 is a linear graph depicting the effect in tumor size (mm³) as a function of days of chemotherapy treatment followed by IPI-926 treatment on day 5 (D5) and day 15 (D15).

FIG. 3A is a bar graph depicting the change in human Indian hedgehog (IHH) expression in naïve, vehicle-treated and IPI-926-treated tumors.

FIG. 3B is a bar graph depicting expression of murine Gli-1 in naïve, vehicle-treated control, and after treatment with IPI-926.

FIGS. 4A-4B show bar graphs depicting increased expression of human Sonic hedgehog (SHH) after chemotherapy with Gemcitabine and Doxorubicin, respectively.

FIGS. 4C-4D are photographs of Western blots from samples after chemotherapy with Gemcitabine and Doxorubicin, respectively. SHH protein is indicated by the arrow having a molecular weight of about 19 kDa.

FIGS. 5A-5B are bar graphs showing modulation of mGLI-1 mRNA in primary xenograft model of ovarian cancer in response to IPI-926.

FIG. 6 shows a maintained decrease in ovarian tumor volume (%) after administration of IPI-926 following carboplatin/taxol chemotherapy.

FIG. 7 are linear graphs showing the effect in tumor size (mm³) as a function of days of post implantation of LuCaP35V (Castration Resistant) in a primary prostate cancer model. The following samples are shown: Vehicle control (administered orally once a day), 40 mg/kg of IPI-926 (administered orally once a day), docetaxel (administered intravenously Q14D for 28 days), or docetaxel (administered intravenously Q14D for 28 days) followed by 40 mg/kg of IPI-926.

FIG. 8A shows a photograph of immunohistochemical staining (IHC) of sections of non-small cell lung cancer for detecting Sonic hedgehog (SHH) ligand.

FIG. 8B is a bar graph depicting murine GLI-1 mRNA expression in lung tumor samples treated with IPI-926 in combination with Gefitinib, Gefitinib-vehicle, and vehicle.

FIG. 9 are linear graphs depicting the activity of IPI-926 in H1650 lung cancer xenograft following treatment with Gefitinib. The following samples are shown: Vehicle control; 40 mg/kg of Gefitinib administered orally for one week; 40 mg/kg of Gefitinib administered orally for one week followed by vehicle control; and 40 mg/kg of Gefitinib administered orally for one week followed by IPI-926 (administered once a day for three weeks). These data indicate the efficacy of IPI-926 following tyrosine kinase treatment in a mutant EGFR non small cell lung cancer model of minimal residual disease.

FIGS. 10A-10B are linear graphs depicting the quantification on log and linear scale, respectively, normalized on each day to the average of vehicle treated animals showing that treatment with IPI-926 for 14 days prior to implant of L3.6pl cells significantly reduced the growth and formation of metastasis within the liver.

FIG. 11 shows graphs depicting the overall percent survival observed from each group within Study #1. Treatment with IPI-926 for 14 days prior to implant, doubles the overall survival rate compared to vehicle treated animals.

FIGS. 12A-12B are linear graphs depicting the quantification on log and linear scale, respectively, normalized on each day to the average of vehicle treated animals showing that treatment with IPI-926 for 14 days prior to implant of L3.6pl cells significantly reduced the growth and formation of metastasis within the liver.

FIG. 13 shows graphs depicting the overall percent survival observed from each group within Study #2. Treatment with IPI-926 for 14 days prior to implant, doubles the overall survival rate compared to vehicle treated animals.

FIG. 14 is a bar graph showing inhibition of Gli1 expression of Ptc^{C/C} mouse medulloblastomas in response to IPI-926 administration via intraperitoneal (IP) injection or oral gavage (PO). Medulloblastoma-bearing Ptc^{C/C} and Smo/Smo mice were treated with a single dose of IPI-926 and the expression of sonic hedgehog pathway target gene Gli1 was analyzed in comparison to vehicle-treated controls and normalized to a Gapdh control.

FIG. 15A is a panel of photographs of Ptc1-null mice or wild type mice after days of the indicated treatment (Vehicle, IPI-926 and wild type).

FIG. 15B is a panel of photographs showing a dramatic change in gross pathology in response to IPI-926 after days of the indicated treatment (Vehicle, IPI-926 and wild type).

FIG. 15C is a panel of ex vivo images with Tumor Paint (Ctx-Cy5.5) after days of the indicated treatment (Vehicle, IPI-926 and wild type).

FIG. 15D is a panel of haematoxylin and eosin (H&E) stained tissue sections after days of the indicated treatment (Vehicle, IPI-926 and wild type).

FIG. 16 is a graph showing the overall survival as a function of time in days from Kaplan-Meier analysis demonstrating that all mice treated with daily IPI-926 for six weeks (line shown as #1) survived, while all vehicle-treated (line shown as #2) mice succumbed to their disease (P<0.001, P value).

FIGS. 17A-17B depict an image panel summarizing a comparison of tissue sections from brains processed outside of the skull or from within the skull (FIG. 17A), and the related 3D renderings of cerebellar or tumor volume (FIG. 17B).

FIG. 18 depict graphs showing estimated tumor volumes (mm³) at each time point for vehicle treated (n=5) and IPI-926 treated Ptc^{C/C} mice (n=7). Note that none of the vehicle-treated mice survived until the 6 week imaging time point.

FIG. 19A shows the overall survival as a function of time in days from Kaplan-Meier analysis of Ptc^{C/C} mice symptomatic for medulloblastoma treated with vehicle (line #3) or intraperitoneal IPI-926 (20 mg/kg/dose) for six weeks (n=24), and were then taken off the drug (n=12; line #2), or given maintenance dosing (20 mg/kg twice per week) for six additional weeks (n=12; line #1).

FIG. 19B is a bar graph depicting intracranial-to-flank tumor take rates from either drug-naïve Ptc^{C/C} tumors or Ptc^{C/C} tumors from mice treated with IPI-926 for 6 weeks and the tumor take rates (P values were generated using Fisher's exact test).

FIG. 19C is a linear graph showing the average tumor volumes (intracranial to flank allograft tumor response) with IPI-926-treated donor (line #1), IPI-926 naïve donor (line #2), and vehicle (line #3), with error bars representing +/−SEM.

FIG. 19D is a linear graph showing the average Gli-luciferase reporter activity in C3H10T1/2 cells transfected with wild type SMOOTHENED (SMO) (squares) or the D473H SMO mutant (triangles) after treatment with various doses of IPI-926.

FIG. 20A is a bar graph depicting a decrease in the initial reduction in Gli1 expression seen in response to daily IPI-926 (20 mg/kg/dose) after 6 weeks of daily treatment. Bars represent the average fold change in Gli1 expression normalized to vehicle-treated controls using n=3 per group, with error bars representing +/−SEM.

FIG. 20B shows tissue sections from mice receiving daily IPI-926 (20 mg/kg) for 3 days or 6 weeks and vehicle controls stained with antibodies recognizing Gli1 (upper panels), Pgp (lower panels) and BCRP (data not shown).

FIG. 20C is a bar graph depicting the relative intensity quantified via imageJ program to evaluate expression of the ABC transporter pump Pgp after prolonged IPI-926 treatment.

FIG. 20D is a series of panels depicting the results of double immunofluorescence analysis showing that most of the cells expressing Gli1 also express Pgp, indicating that hedgehog pathway activity is maintained in cells with active ABC transporters.

FIG. 21-22 is a schematic of the experimental design for Example 6.

FIG. 23 is a linear graph showing the effect of IPI-926 on post tumor debulking in a primary xenograft model of SCLC. Tumors were established and treated with etoposide/cisplatin followed by vehicle or IPI-926. Similar results are described in Example 2, above. Thus, IPI-926 is shown to be efficacious post-chemotherapy in a primary SCLC model of MRD.

FIG. 24 is a linear graph showing the effect of IPI-926 on post tumor debulking in a xenograft model of mutant EGFR NSCLC. Tumors were established and treated with gefitinib followed by vehicle or IPI-926. Similar results are described in Example 3, above. Thus, IPI-926 is shown to be efficacious post-tyrosine kinase inhibition (TKI) in a mutant EGFR NSCLC model of MRD.

FIG. 25 is a linear graph showing the effect of IPI-926 on post tumor debulking in a primary xenograft model of castrate-resistant prostate cancer. Tumors were established and treated with docetaxel followed by vehicle or IPI-926. Similar results are described in Example 3, above. Thus, IPI-926 is shown to be efficacious post-chemotherapy in an MRD model of castrate-resistant prostate cancer.

FIG. 26 is a line graph the effect of IPI-926 post-tumor debulking as assessed using a primary xenograft model of serous ovarian cancer. Tumors were established and treated with taxol/carboplatin followed by vehicle or IPI-926. These data indicate that IPI-926 displays efficacy post-chemotherapy in a minimal residual disease model of primary serous ovarian cancer.

FIG. 27 is a line graph depicting Gli-1 levels (as assessed by RT-PCR) in tumor-associated stroma dissected from tumor samples of 19 patients with high grade serous ovarian cancer. These data indicate that elevated Gli-1 expression in stroma from serous ovarian cancer patients is associated with worsened survival.

### DETAILED DESCRIPTION

Hedgehog signaling has been associated with several ligand-independent and ligand-dependent cancerous conditions. Ligand-independent cancerous conditions can be associated with a genetic mutation in a component of the hedgehog pathway (e.g., a hedgehog receptor such as Smoothened (Smo) or Patched (Ptc)) that leads to abnormal receptor expression and/or activity. Without being bound by theory, inhibition (e.g., by direct inhibition) of aberrant activation of a hedgehog receptor, e.g., Smo and/or Ptc, can be used to treat or prevent conditions associated with ligand-independent hedgehog activation (e.g., by decreasing or inhibiting oncogenic signaling and/or inducing tumor cell apoptosis). Examples of cancerous conditions involving genetic mutations in a hedgehog receptor that can be treated with the methods of the invention include basal cell carcinoma (BCC) and medulloblastoma.

Ligand-dependent cancerous conditions can involve paracrine signaling mechanisms (e.g., between a hedgehog-secreting tumor and the tumor microenvironment, e.g., the surrounding stroma). For example, a hedgehog ligand is secreted from a tumor cell and activates a hedgehog receptor (e.g., Smo and/or Ptc) in the tumor microenvironment (e.g., a nearby stromal cell). Without being bound by theory, hedgehog inhibition in this context is believed to cause one or more of: (i) depleting or reducing desmoplastic stroma and/or fibrosis; (ii) increasing the vascularity of the tumor; or (iii) rendering the tumor more accessible to chemotherapy. Examples of paracrine cancerous conditions that can be treated or prevented with the methods of the invention include desmoplastic tumors, cancers of the pancreas, small cell lung cancer (SCLC), ovary, prostate and bladder.

Ligand-dependent cancerous conditions can also involve direct signaling by a hedgehog ligand to the tumor or cancer cell. Without being bound by theory, inhibition (e.g., direct inhibition) of hedgehog-mediated activation of a hedgehog receptor, e.g., autologous activation of Smo and/or Ptc) can be used to treat or prevent conditions associated with ligand-dependent hedgehog activation of a tumor cell. Examples of such cancerous conditions include, but are not limited to sarcomas, chondrosarcoma, osteosarcoma, heme malignancies, chronic myelogenous leukemia (CML), SCLC, multiple melanoma (MM), chronic lymphocytic leukemia (CLL), acute lymphoblastic leukemia (ALL), and acute myelogenous leukemia (AML).

Sonic Hedgehog (SHH) expression is detected in a wide number of primary tumors and xenograft models, including pancreatic ductal carcinomas (about 70% positive immunostaining), colon adenocarcinoma (about 84% positive immunostaining), ovarian cystadenocarcinoma (about 44% positive immunostaining) and prostate adenocarcinoma (about 77% positive immunostaining). Numerous xenograft tumor models show both SHH expression and suppression of the hedgehog signaling mediator Gli-1 in the murine stroma in response to treatment with the hedgehog inhibitor, IPI-926 (also referred to herein as a “compound of formula 32”).

Genetically engineered mouse models of cancer provide an alternative to transplantation models for preclinical therapeutic evaluation. KPC mice are designed to conditionally express endogenous mutant Kras and p53 alleles in pancreatic cells, resulting in focal tumors that mimic the pathophysiological and molecular aspects of pancreatic cancer. KPC mice treated with a combination of the hedgehog inhibitor, IPI-926, and the therapeutic agent, Gemcitabine, have shown to produce a transient increase in intratumoral vascular density and intratumoral concentration of gemcitabine, leading to transient stabilization of disease (Olive et al. (2009) Science 324 (5933) 1457-1461). IPI-926 appears to enhance the delivery of therapeutic agents (e.g., Gemcitabine or doxorubicin) to the tumor, e.g., presumably through decreased desmoplasia and/or increased perfusion. This finding is detected in other tumor models. For example, in L3.6pl xenografts, administration of IPI-926 enhances the therapeutic agent effect of a formulation of paclitaxel bonded to albumin (Abraxane®).

In one embodiment, Applicants have discovered that hedgehog inhibitors (e.g., IPI-926) can be used following cyto-reductive chemotherapy, particularly when administered either concurrently with chemotherapy (e.g., having at least some period of overlap between the therapeutic agent regimen and the administration of the hedgehog inhibitor), or without a substantial delay after cessation of cancer therapy (e.g., simultaneously with, or less than 15, 10, 8, 6, 5, 4, 3 days, or less than 48, 36, 24, 14, 10, 9, 8, 7, 6, 5, 4, 3, 2, or 1 hour after cessation of the cancer therapy). In related embodiments, the hedgehog inhibitor(s) (e.g., IPI-926) have been shown to be effective in maintenance therapy of a wide number of chemoresponsive tumor types, including ovarian cancer, prostate cancer and non-small cell lung cancer (Example 3). More specifically, the efficacy of IPI-926 was evaluated when applied as maintenance therapy following chemotherapy of a xenograft primary ovarian cancer with carboplatin/taxol, prostate cancer with docetaxel, and non-small cell lung cancer model with the tyrosine kinase inhibitor, Gefitinib.

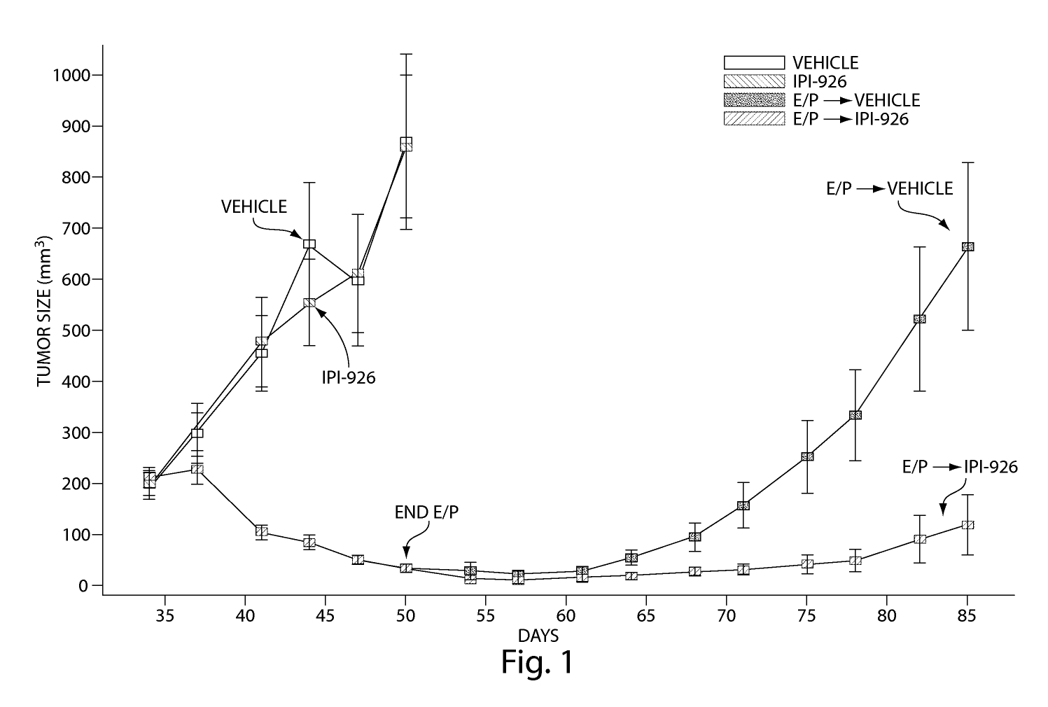

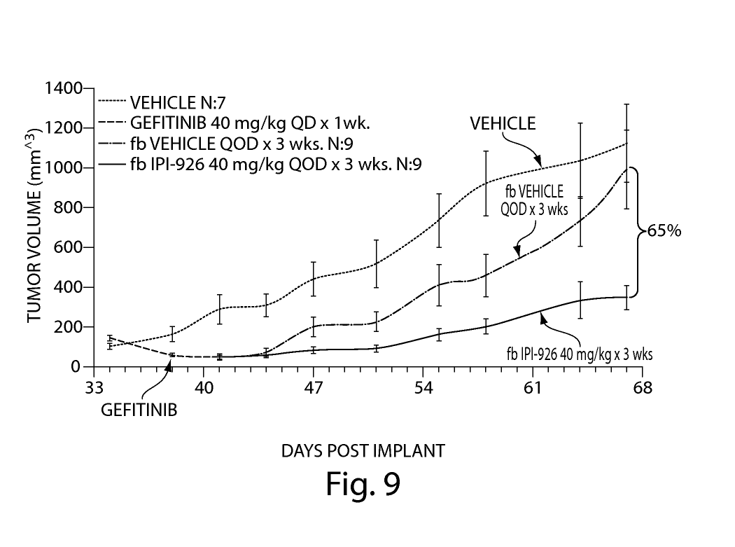

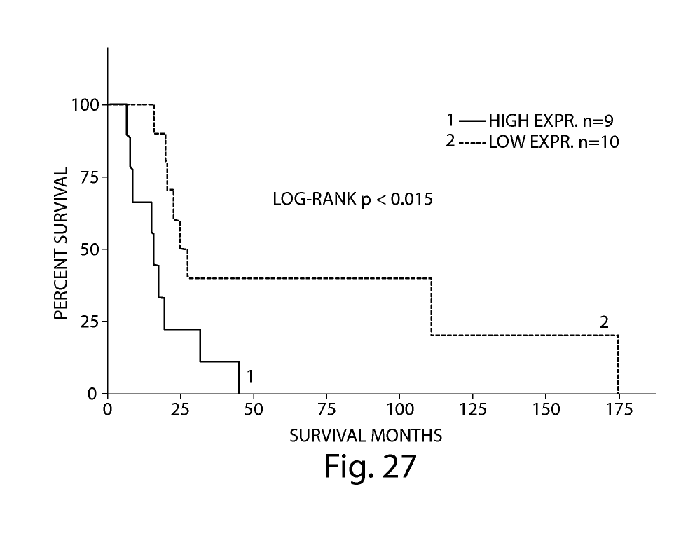

In other embodiments, Applicants have demonstrated that the hedgehog inhibitor, IPI-926, shows anti-tumor activity post-cytoreduction with either standard of care chemotherapy or targeted therapy, in multiple pre-clinical models for minimal residual disease (MRD). For example, IPI-926 has been shown to be efficacious in multiple pre-clinical MRD models, including post-chemotherapy in a primary SCLC model of MRD (FIG. 1), post-tyrosine kinase inhibitor treatment in a mutant EGFR NSCLC model of MRD (FIG. 9), post-chemotherapy in a primary MRD model of castrate-resistant prostate cancer (FIG. 7), and post-chemotherapy in a MRD model of primary serous ovarian cancer (FIG. 26). Elevated expression levels of Gli-1 in stroma from serous ovarian cancer patients was associated with worsened survival (FIG. 27). Taken together these results demonstrate that IPI-926 can be used as post cytoreductive therapy.

In yet other embodiments, Applicants have shown that pre-treatment of a subject with a hedgehog inhibitor (e.g., IPI-926) reduces the formation and growth of metastatic tumors, leading to a reduction in tumor burden and increased survival (Example 4).

Without being bound by theory, it is believed that the hedgehog inhibitor (e.g., IPI-926) reduces the tumor ability to reestablish itself after therapy or establish anew. The hedgehog inhibitor (e.g., IPI-926) is believed to inhibit or reduce one or more of: the stroma to which metastatic cells seed; angiogenic mechanisms associated with solid tumor growth and maintenance; and/or minimal residual disease. In one embodiment, one or more hedgehog inhibitors are used to treat a cancer that shows at least 10%, 20%, 30%, 40%, 50%, 60%, 70%, 80%, 90% or more tumor shrinkage in response to chemotherapy, radiation, and/or surgery. In other embodiments, the one or more hedgehog inhibitors reduce minimal residual disease.

“Minimal residual disease” or “MRD” refers to the presence of residual malignant cells after a primary therapy, e.g., chemotherapy, radiation therapy, surgery, and/or targeted therapy. Typically, the cancer cells in a subject with MRD are present in small numbers, and are difficult to find by routine means. Residual tumor cells can lead to disease recurrence and shortened survival.

Accordingly, the present invention relates to new therapeutic regimens that optimize the benefits of hedgehog inhibition. In one embodiment, methods for treating one or more hedgehog-associated cancers, e.g., ligand-dependent and ligand-independent cancers, by administering a hedgehog inhibitor(s), alone or in combination with another cancer therapy, e.g., one or more therapeutic agents, radiation therapy and/or surgery, are disclosed. The hedgehog-associated cancer can be a cancer (e.g., a cancer chosen from one or more of: lung cancer (e.g., small cell lung cancer or non-small cell lung cancer), pancreatic cancer, prostate cancer, bladder cancer, ovarian cancer, breast cancer, colon cancer, multiple myeloma, acute myelogenous leukemia (AML), chronic myelogenous leukemia (CML), neuroendocrine cancer, or chondrosarcoma. In one embodiment, the cancer therapy and the one or more hedgehog inhibitors are administered concurrently or sequentially (for example, the hedgehog inhibitor is administered after, or close to completion of another cancer therapy). The hedgehog inhibitor can be administered concurrently with chemotherapy (e.g., having at least some period of overlap between the therapeutic agent regimen and the administration of the hedgehog inhibitor). For example, the hedgehog inhibitor(s) can be administered prior to cessation of the cancer therapy (e.g., at least 1, 2, 3, 4, 5, 10, 15, 24, 36, or 48 hours; at least 1, 2, 3, 4, 5, 6, 7, 10, 14, or days; at least 1, 2, 3, 4, 5, 6, 8, 10, or 12 months; prior to cessation of cancer therapy). The hedgehog inhibitor can also be administered without a substantial delay after cessation of cancer therapy (e.g., simultaneously with, or less than 15, 10, 8, 6, 5, 4, 3 days, or less than 48, 36, 24, 14, 10, 9, 8, 7, 6, 5, 4, 3, 2, or 1 hour after cessation of the cancer therapy).

In other embodiments, the hedgehog inhibitor(s) is administered to a cancer patient after cessation of another cancer therapy (e.g., tyrosine kinase inhibition), such as one or more therapeutic agents, radiation therapy and/or surgery. In other embodiments, the hedgehog inhibitor(s) is administered to a subject (e.g., a cancer patient) as maintenance therapy (e.g., as a prolonged or extended therapy after cessation of another cancer treatment). The hedgehog-associated cancer treated can be a cancer patient substantially or completely in remission from a cancer (e.g., a cancer chosen from one or more of: lung cancer (e.g., small cell lung cancer or non-small cell lung cancer), pancreatic cancer, prostate cancer, bladder cancer, ovarian cancer (e.g., serous ovarian cancer), breast cancer, colon cancer, multiple myeloma, acute myelogenous leukemia (AML), chronic myelogenous leukemia (CML) and neuroendocrine cancer). In certain embodiments, the subject has a minimal residual disease.

In one embodiment, the hedgehog inhibitor(s) is administered at a diminished dose from a first line therapeutic dose (e.g., a first line therapeutic dose administered to a subject who has not been previously administered another drug intended to treat the cancer).

In yet another embodiment, methods to treat or prevent a metastasis or metastatic growth, e.g., liver metastasis, by administering to a subject (e.g., a cancer patient) one or more hedgehog inhibitors are disclosed. In one embodiment, the one or more hedgehog inhibitors are administered prior to detection of a metastatic lesion. In other embodiments, a subject having a localized cancer is treated with one or more hedgehog inhibitors (e.g., IPI-926) to reduce the formation and growth of metastatic tumors, leading to a reduction in tumor burden and increased survival.

In other embodiments, methods to reduce minimal residual disease are disclosed. In one embodiment, one or more hedgehog inhibitors are used to treat a cancer that shows at least 10%, 20%, 30%, 40%, 50%, 60%, 70%, 80%, 90% or more tumor shrinkage in response to chemotherapy, radiation, and/or surgery.

Various aspects of the invention are described in further detail below. Additional definitions are set out throughout the specification.

Hedgehog Inhibitors

Suitable hedgehog inhibitors for use with the present invention include, for example, those described and disclosed in U.S. Pat. No. 7,230,004, U.S. Patent Application Publication No. 2008/0293754, U.S. Patent Application Publication No. 2008/0287420, and U.S. Patent Application Publication No. 2008/0293755, the entire disclosures of which are incorporated by reference herein.

Examples of other suitable hedgehog inhibitors include those described in U.S. Patent Application Publication Nos. US 2002/0006931, US 2007/0021493 and US 2007/0060546, and International Application Publication Nos. WO 2001/19800, WO 2001/26644, WO 2001/27135, WO 2001/49279, WO 2001/74344, WO 2003/011219, WO 2003/088970, WO 2004/020599, WO 2005/013800, WO 2005/033288, WO 2005/032343, WO 2005/042700, WO 2006/028958, WO 2006/050351, WO 2006/078283, WO 2007/054623, WO 2007/059157, WO 2007/120827, WO 2007/131201, WO 2008/070357, WO 2008/110611, WO 2008/112913, and WO 2008/131354.

Additional examples of hedgehog inhibitors include, but are not limited to, GDC-0449 (also known as RG3616 or vismodegib) described in, e.g., Von Hoff D. et al., N. Engl. J. Med. 2009; 361(12):1164-72; Robarge K. D. et al., Bioorg Med Chem Lett. 2009; 19(19):5576-81; Yauch, R. L. et al. (2009) Science 326: 572-574; Sciencexpress: 1-3 (10.1126/science.1179386); Rudin, C. et al. (2009) New England J of Medicine 361-366 (10.1056/nejma0902903); BMS-833923 (also known as XL139) described in, e.g., in Siu L. et al., J. Clin. Oncol. 2010; 28:15s (suppl; abstr 2501); and National Institute of Health Clinical Trial Identifier No. NCT006701891; LDE-225 described, e.g., in Pan S. et al., ACS Med. Chem. Lett., 2010; 1(3): 130-134; LEQ-506 described, e.g., in National Institute of Health Clinical Trial Identifier No. NCT01106508; PF-04449913 described, e.g., in National Institute of Health Clinical Trial Identifier No. NCT00953758; Hedgehog pathway antagonists disclosed in U.S. Patent Application Publication No. 2010/0286114; SMOi2-17 described, e.g., U.S. Patent Application Publication No. 2010/0093625; SANT-1 and SANT-2 described, e.g., in Rominger C. M. et al., J. Pharmacol. Exp. Ther. 2009; 329(3):995-1005; 1-piperazinyl-4-arylphthalazines or analogues thereof, described in Lucas B. S. et al., Bioorg. Med. Chem. Lett. 2010; 20(12):3618-22.

In certain embodiments, the hedgehog inhibitor is a compound of formula (I):

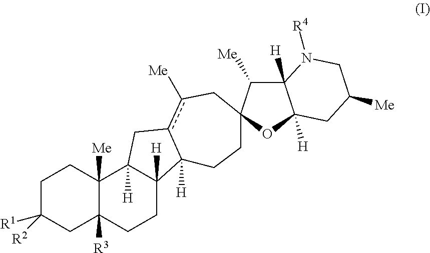

or a pharmaceutically acceptable form thereof (e.g., a salt and/or solvate) thereof; wherein:

R¹ is H, alkyl, —OR, amino, sulfonamido, sulfamido, —OC(O)R⁵, —N(R⁵)C(O)R⁵, or a sugar;

R² is H, alkyl, alkenyl, alkynyl, aryl, cycloalkyl, nitrile, or heterocycloalkyl;

or R¹ and R² taken together form ═O, ═S, ═N(OR), ═N(R), ═N(NR₂), or ═C(R)₂;

R³ is H, alkyl, alkenyl, or alkynyl;

R⁴ is H, alkyl, alkenyl, alkynyl, aryl, cycloalkyl, heterocycloalkyl, aralkyl, heteroaryl, heteroaralkyl, haloalkyl, —OR, —C(O)R⁵, —CO₂R⁵, —SO₂R⁵, —C(O)N(R⁵)(R⁵), —[C(R)₂]_{q}—R⁵, —[(W)—N(R)C(O)]_{q}R⁵, —[(W)—C(O)]_{q}R⁵, —[(W)—C(O)O]_{q}R⁵, —[(W)—OC(O)]_{q}R⁵, —[(W)—SO₂]_{q}R⁵, —[(W)—N(R⁵)SO₂]_{q}R⁵, —[(W)—C(O)N(R⁵)]_{q}R⁵, —[(W)—O]_{q}R⁵, —[(W)—N(R)]_{q}R⁵, —W—NR₃⁺X⁻ or —[(W)—S]_{q}R⁵; wherein each W is independently for each occurrence a diradical such as an alkylene; each q is independently for each occurrence 1, 2, 3, 4, 5, or 6; and X⁻ is an anion (e.g., a halide);

each R⁵ is independently for each occurrence H, alkyl, alkenyl, alkynyl, aryl, cycloalkyl, heterocycloalkyl, aralkyl, heteroaryl, heteroaralkyl or —[C(R)₂]ₚ—R⁶; wherein p is 0-6; or any two occurrences of R⁵ on the same substituent can be taken together to form a 4-8 membered optionally substituted ring which contains 0-3 heteroatoms selected from N, O, S, and P; and

each R⁶ is independently hydroxyl, —N(R)COR, —N(R)C(O)OR, —N(R)SO₂(R), —C(O)N(R)₂, —OC(O)N(R)(R), —SO₂N(R)(R), —N(R)(R), —COOR, —C(O)N(OH)(R), —OS(O)₂OR, —S(O)₂OR, —OP(O)(OR)(OR), —NP(O)(OR)(OR), or —P(O)(OR)(OR); and

each R is independently H, alkyl, alkenyl, alkynyl, aryl, cycloalkyl or aralkyl;

provided that when R², R³ are H and R⁴ is hydroxyl; R¹ cannot be hydroxyl;

provided that when R², R³, and R⁴ are H; R¹ cannot be hydroxyl; and

provided that when R², R³, and R⁴ are H; R¹ cannot be sugar.

In certain embodiments, R¹ is H, hydroxyl, alkoxyl, aryloxy, or amino.

In some embodiments, R¹ and R² taken together along with the carbon to which they are bonded, form ═O, ═N(OR), or ═S.

In other embodiments, R³ is H and/or R⁴ is H, alkyl, hydroxyl, aralkyl, —[C(R)₂]_{q}—R⁵, —[(W)—N(R)C(O)]_{q}R⁵, —[(W)—N(R)SO₂]_{q}R⁵, —[(W)—C(O)N(R)]_{q}R⁵, —[(W)—O]_{q}R⁵, —[(W)—C(O)]_{q}R⁵, or —[(W)—C(O)O]_{q}R⁵.

In yet other embodiments, R¹ is H or —OR, R² is H or alkyl, and R⁴ is H.

In yet other embodiments, R² is H or alkyl, R³ is H, alkyl, alkenyl, alkynyl, aryl, cycloalkyl, heterocycloalkyl, or aralkyl; and/or R⁴ is H, alkyl, aralkyl, —[(W)—N(R)C(O)]_{q}R⁵, —[(W)—N(R)SO₂]_{q}R⁵, —[(W)—C(O)N(R)]_{q}R⁵, —[(W)—O]_{q}R⁵, —[(W)—C(O)]_{q}R⁵, or —[(W)—C(O)O]_{q}R⁵.

In yet other embodiments, R¹ is sulfonamido.

Specific examples of hedgehog inhibitors include compounds, or pharmaceutically acceptable salts and/or solvates thereof, described in U.S. Patent Application 2008/0293754 and also provided below in Table 1:

TABLE 1

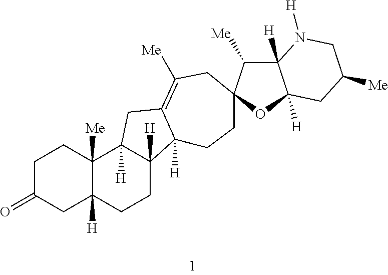

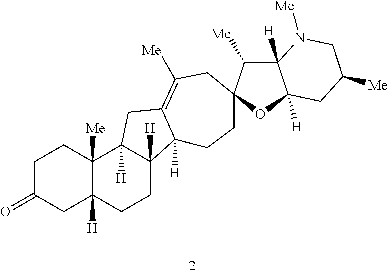

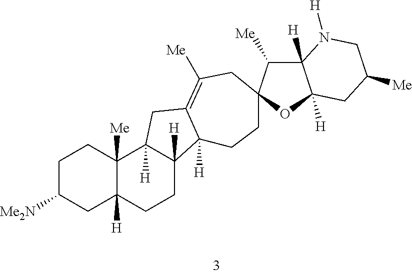

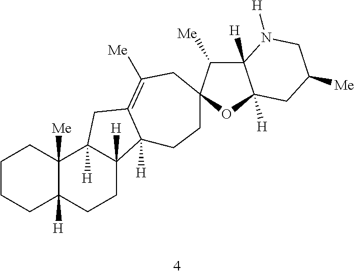

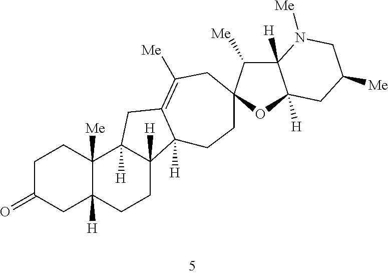

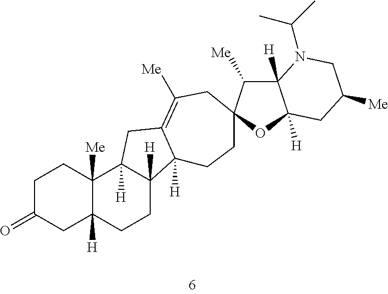

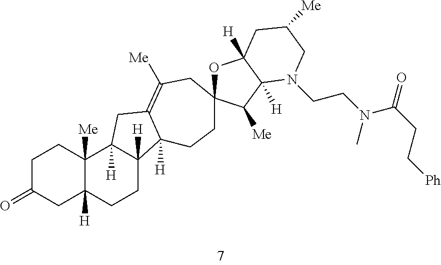

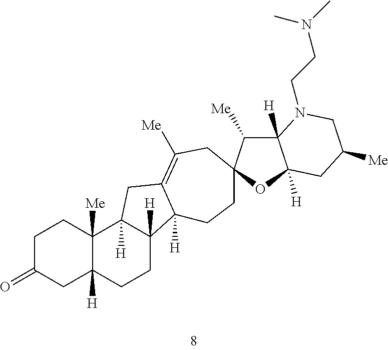

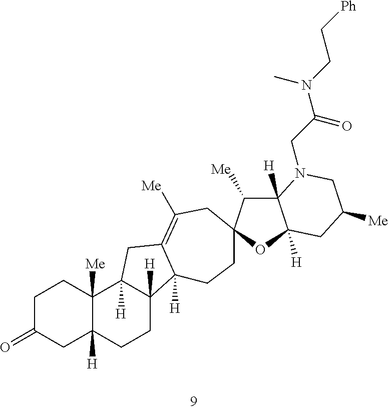

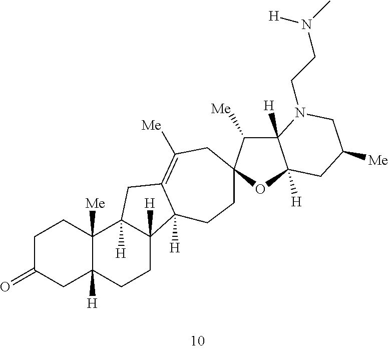

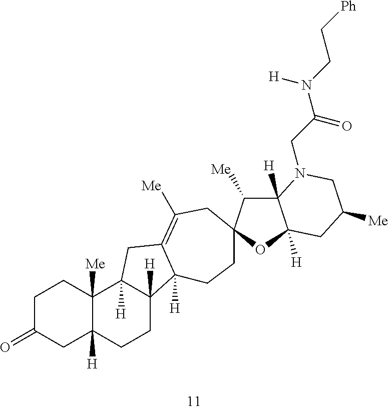

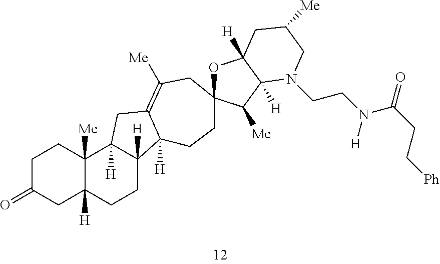

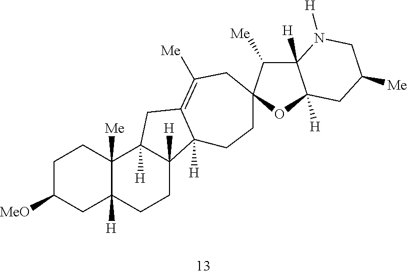

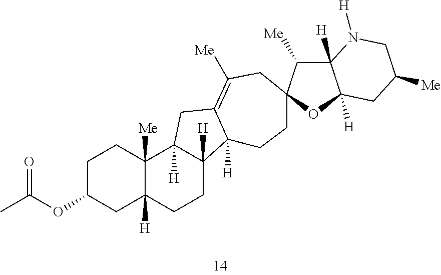

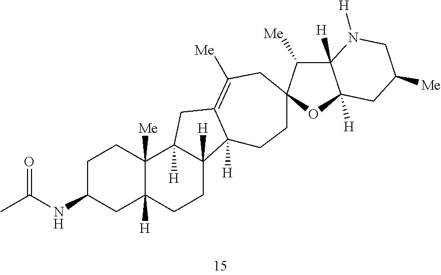

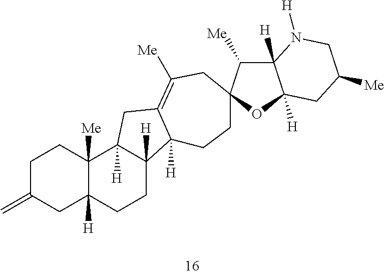

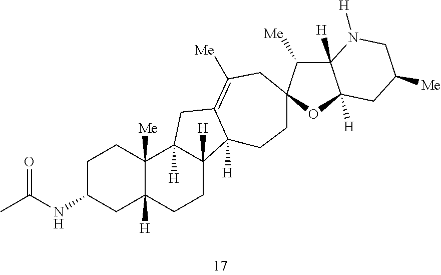

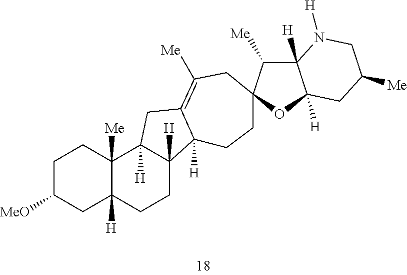

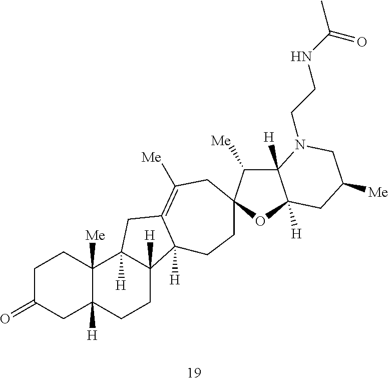

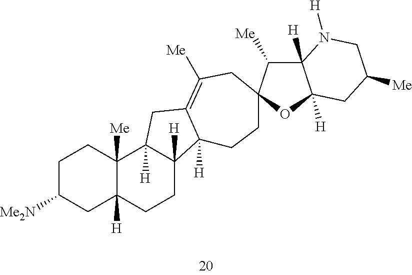

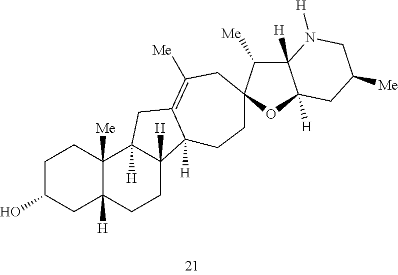

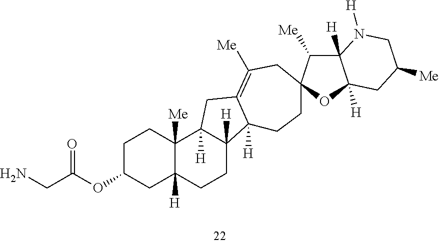

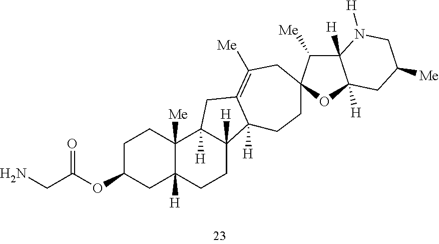

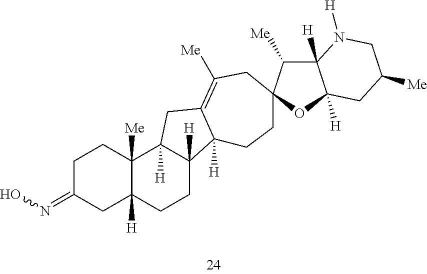

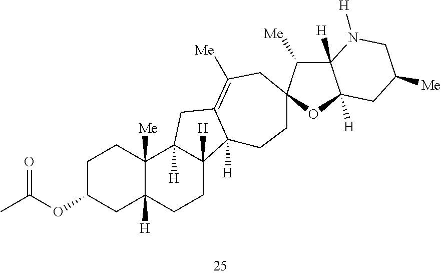

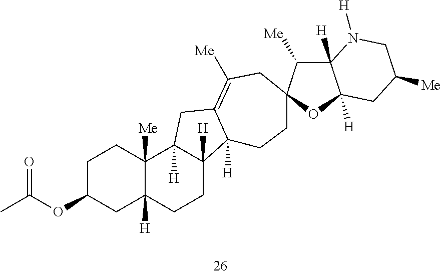

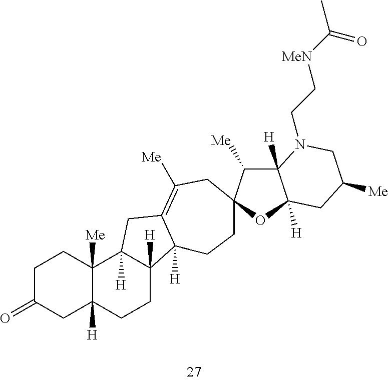

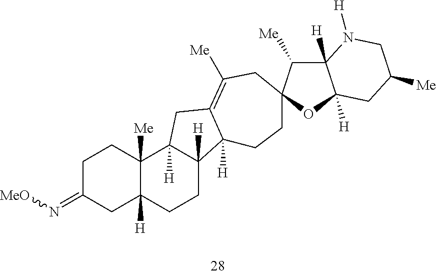

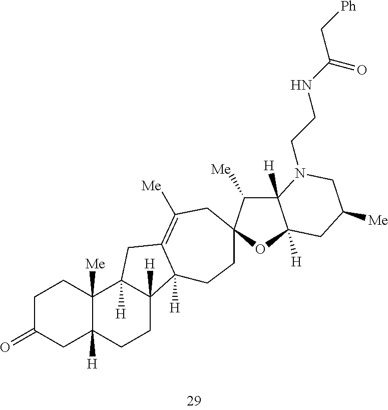

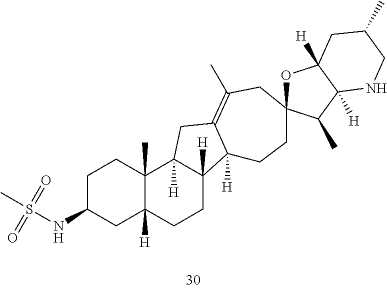

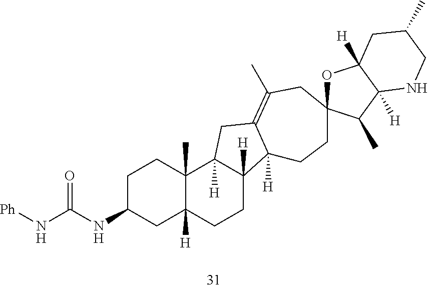

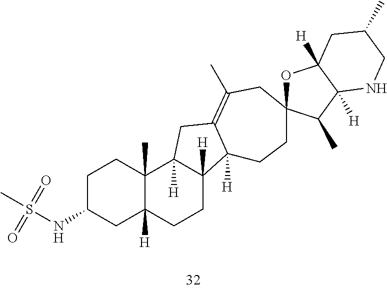

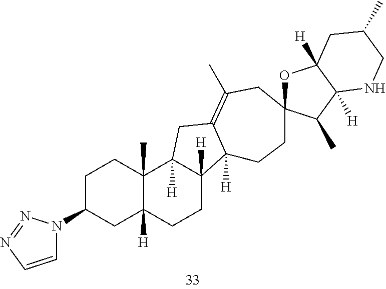

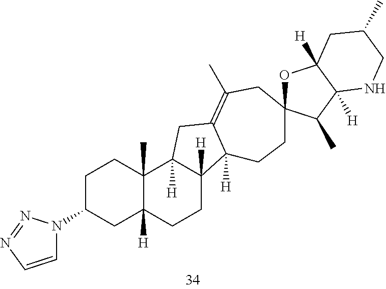

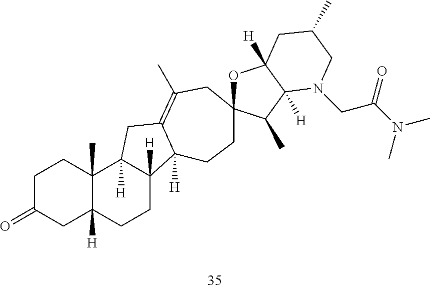

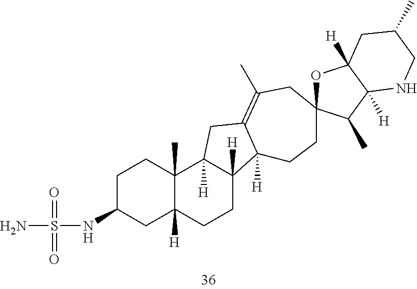

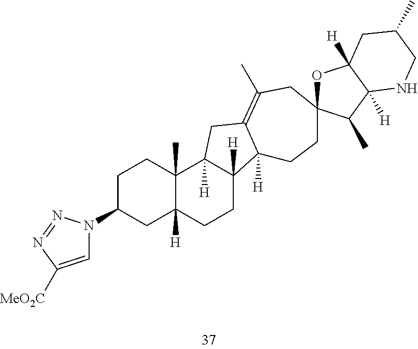

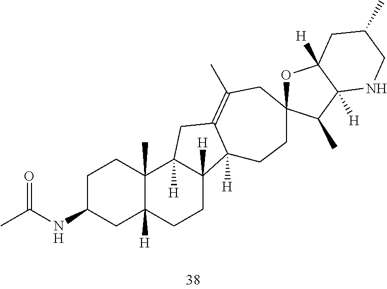

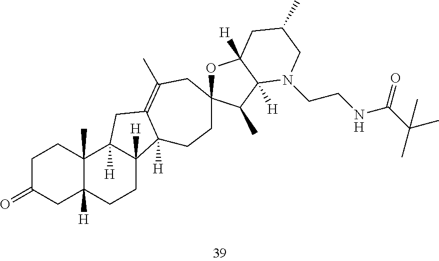

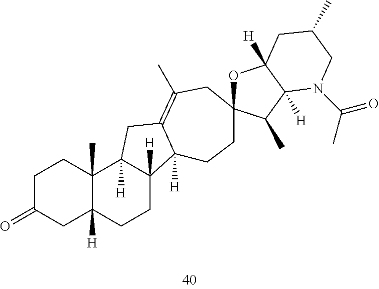

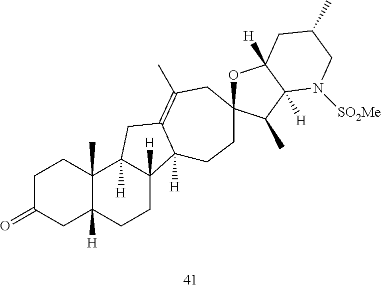

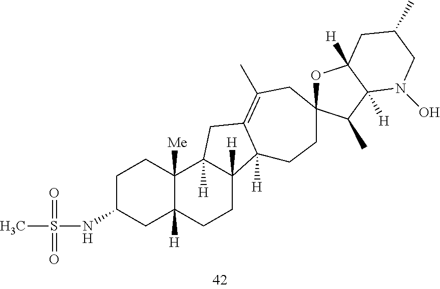

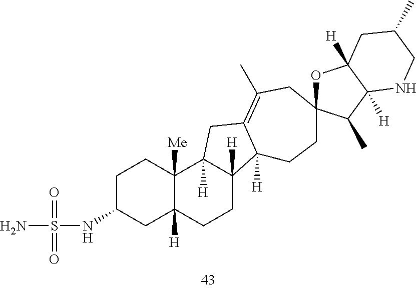

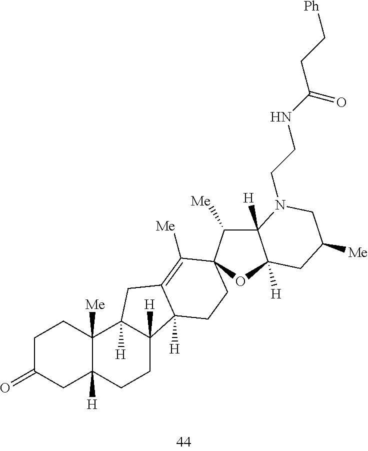

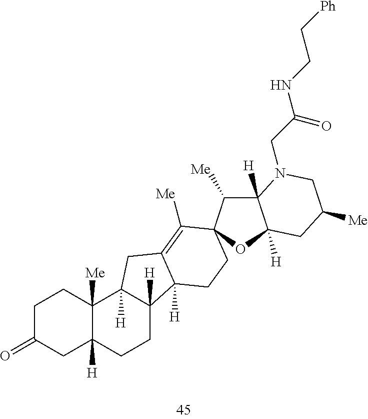

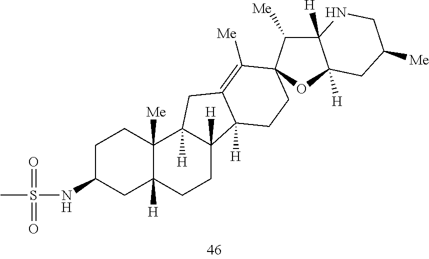

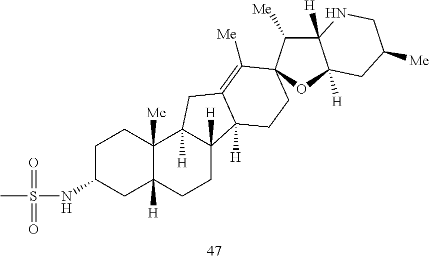

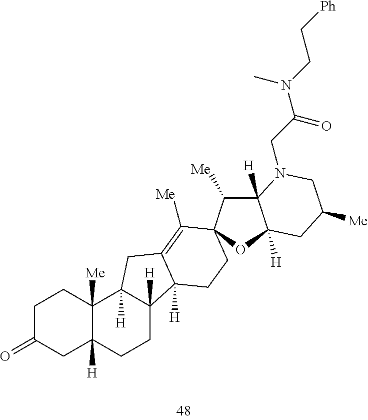

Other examples of hedgehog inhibitors include compounds, or pharmaceutically acceptable salts and/or solvates thereof, described in U.S. Pat. No. 7,230,004 and also provided below in Table 2:

TABLE 2

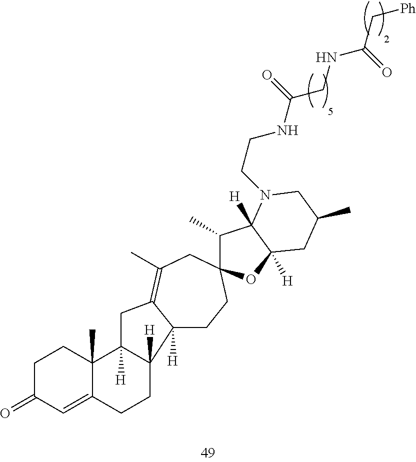

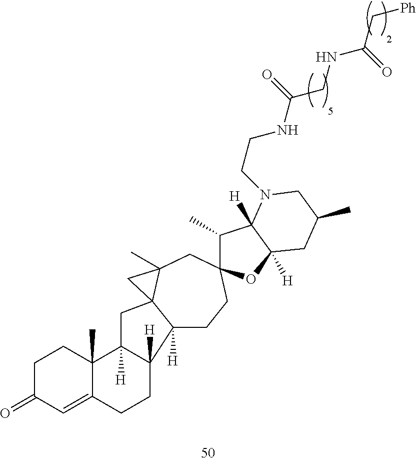

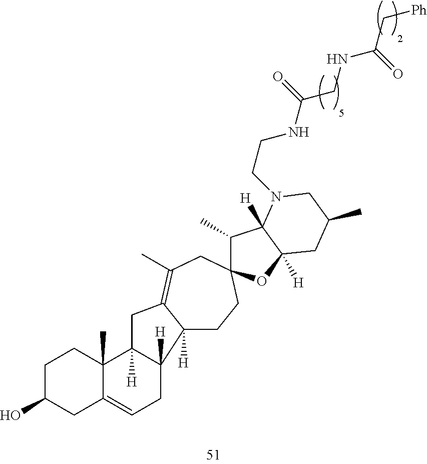

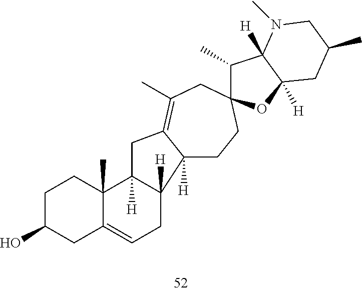

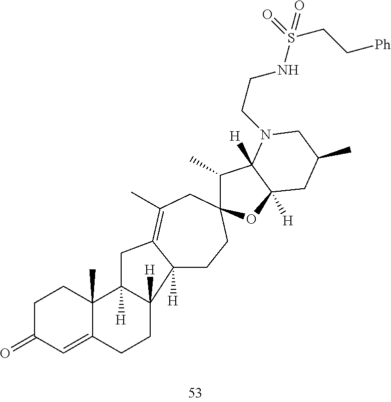

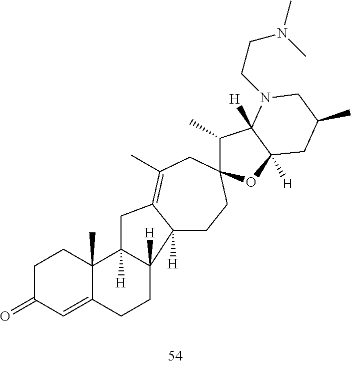

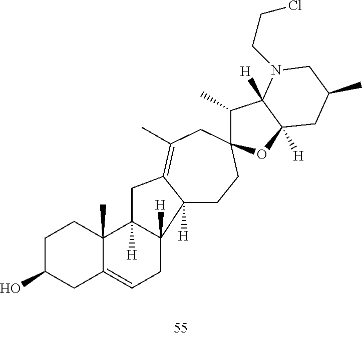

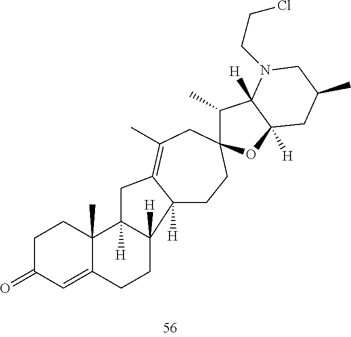

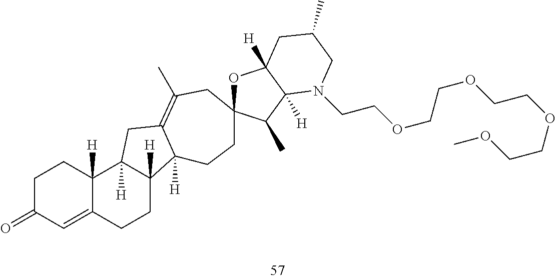

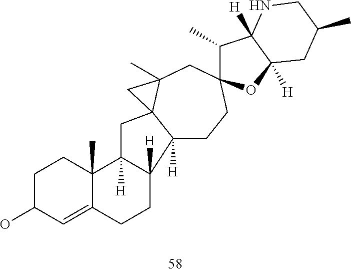

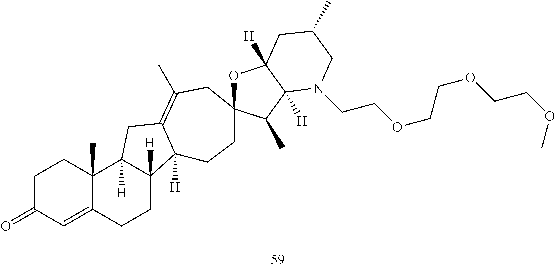

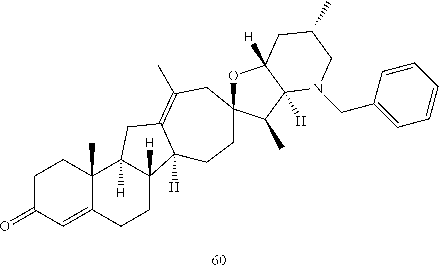

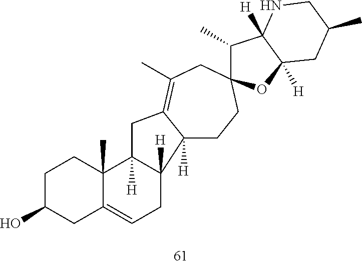

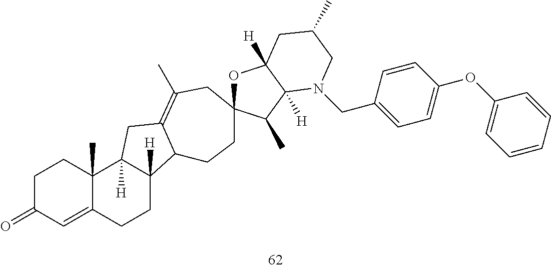

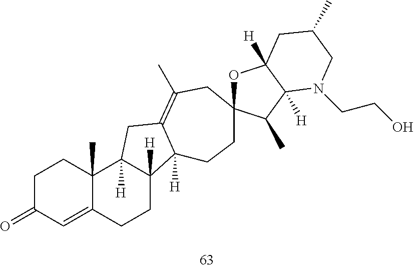

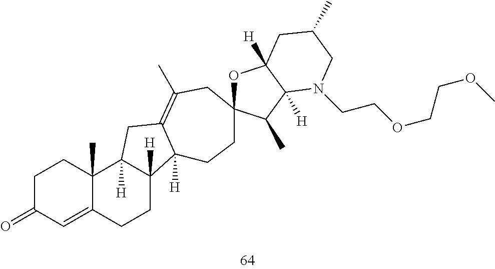

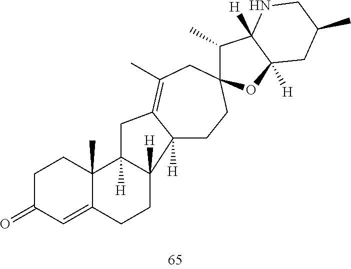

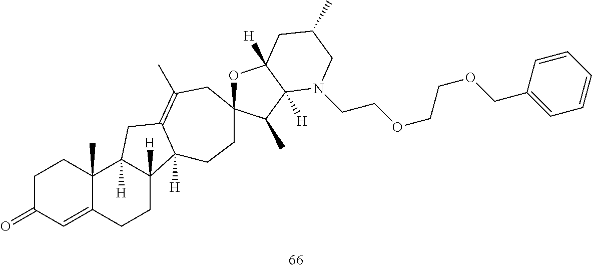

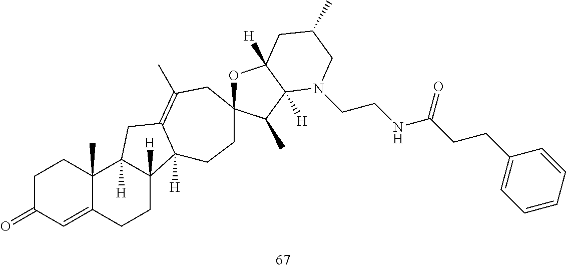

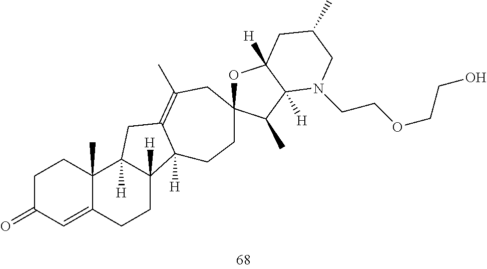

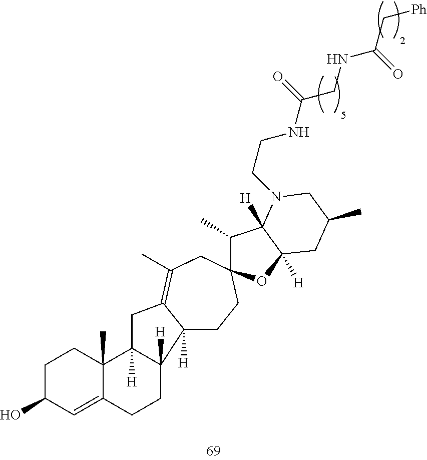

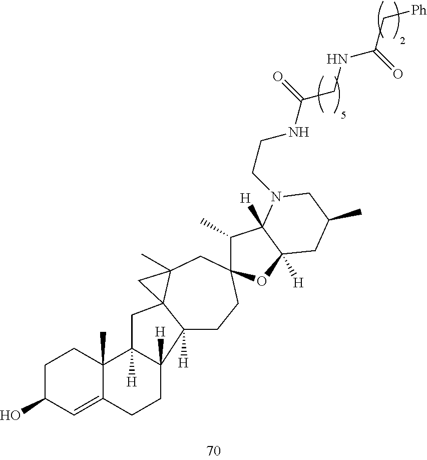

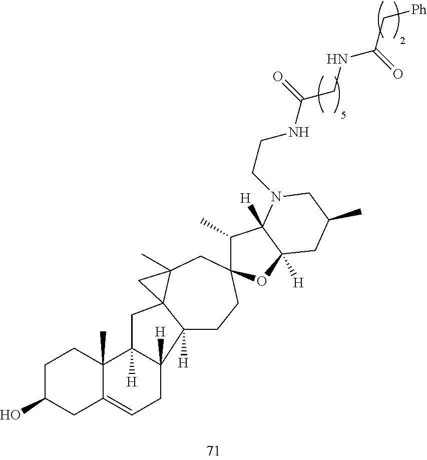

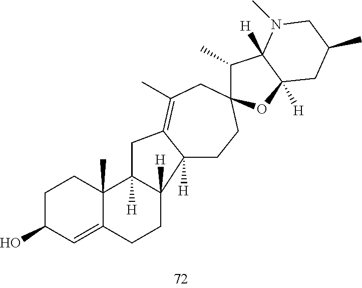

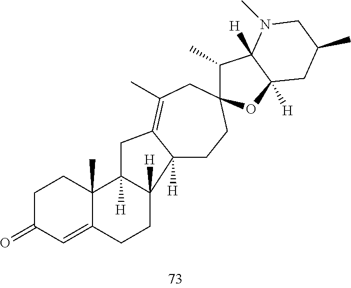

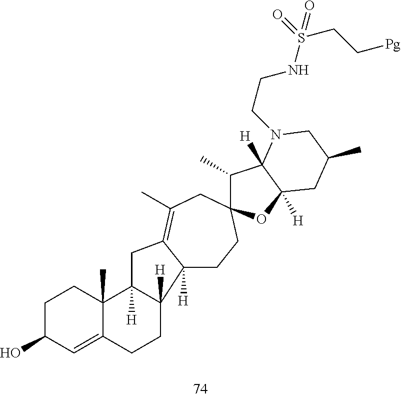

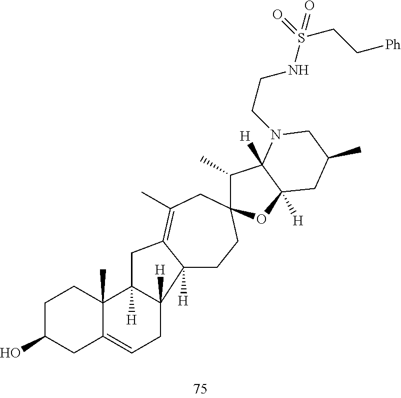

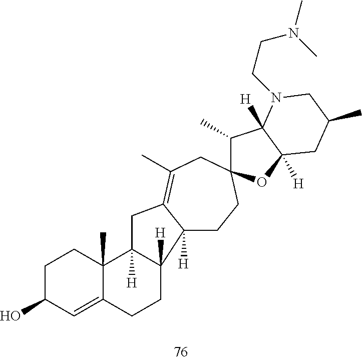

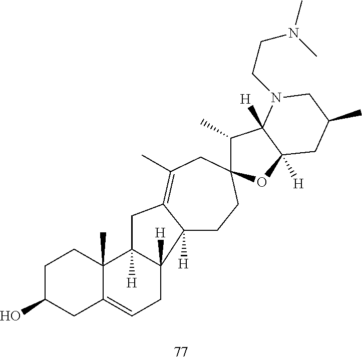

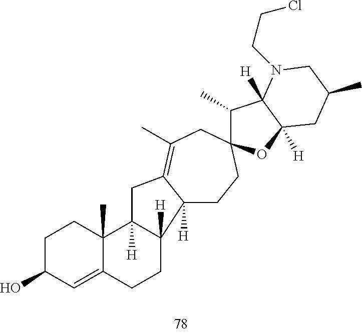

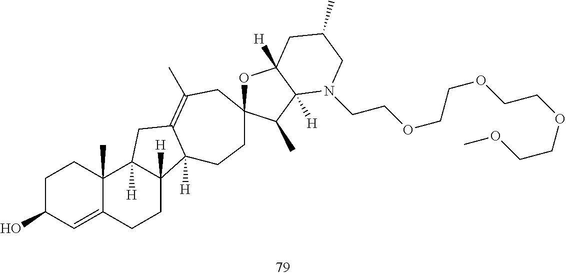

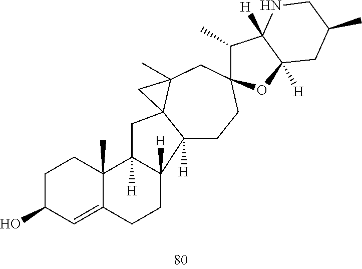

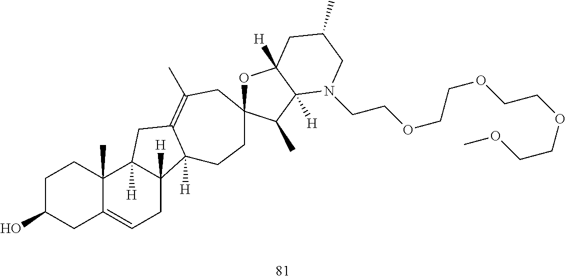

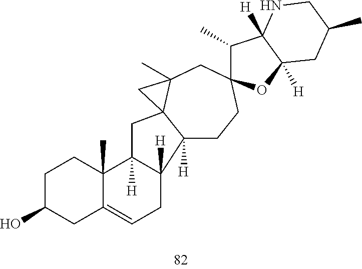

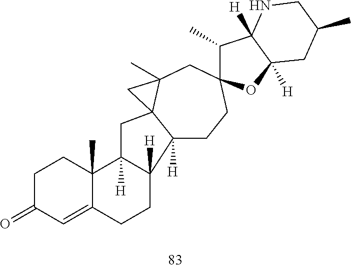

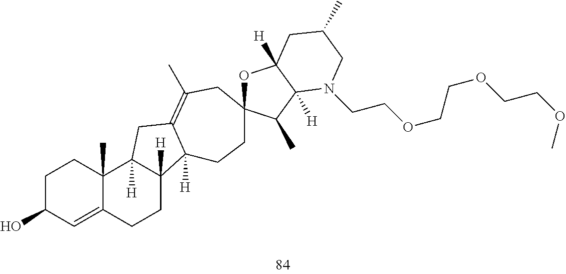

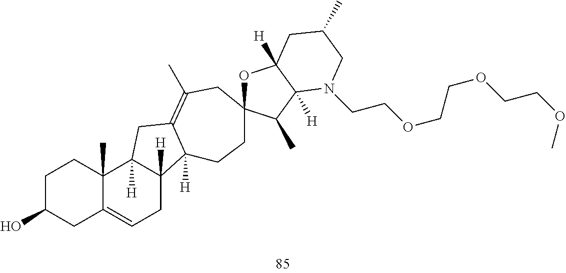

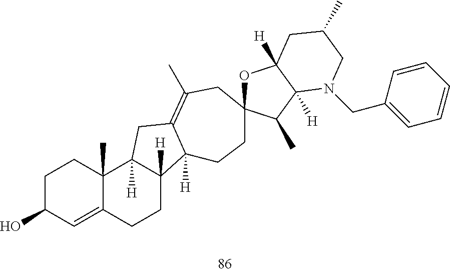

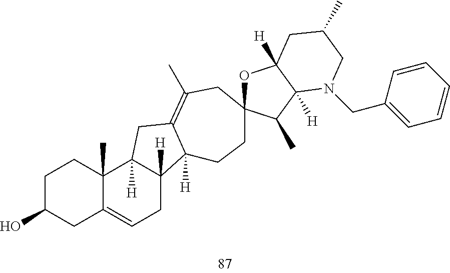

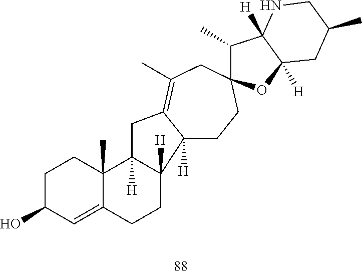

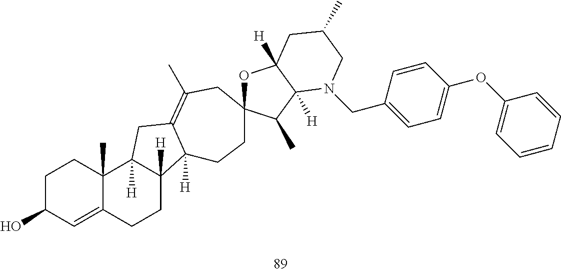

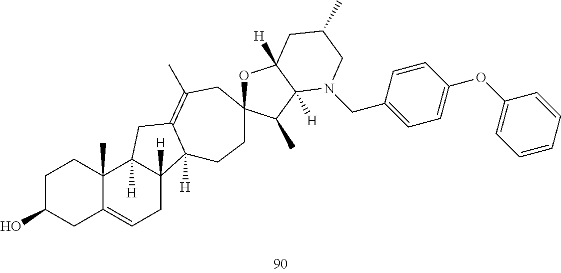

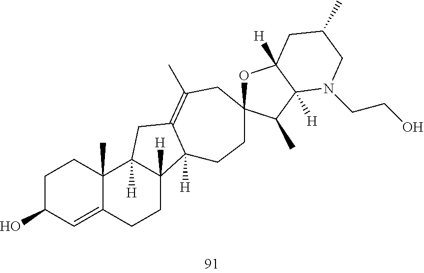

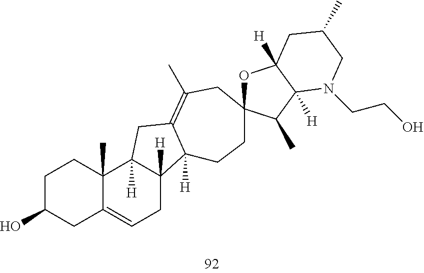

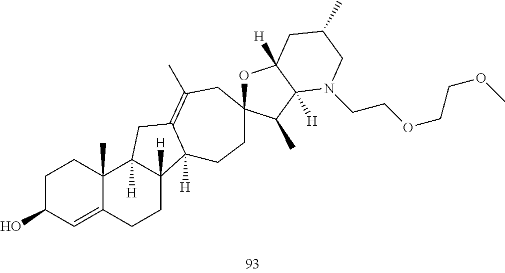

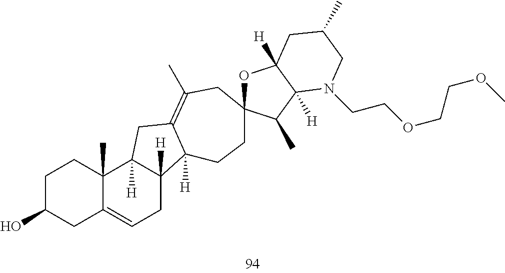

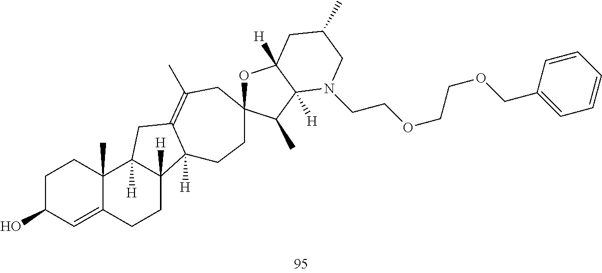

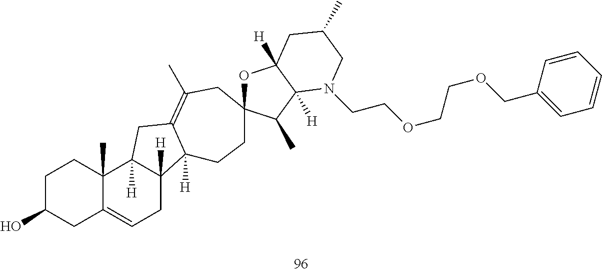

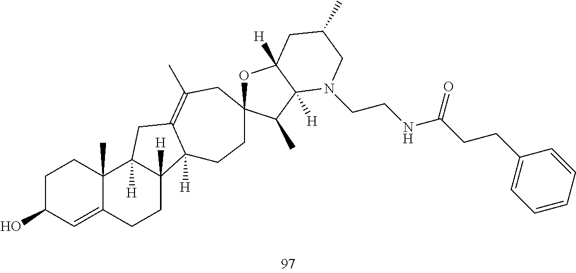

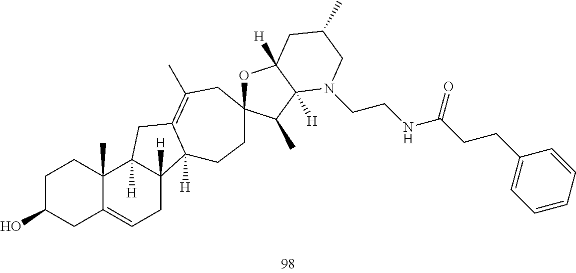

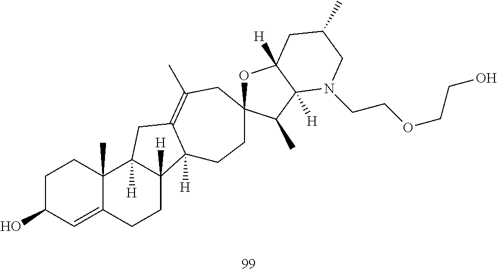

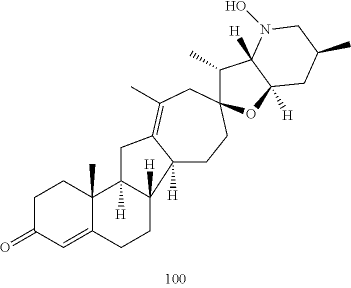

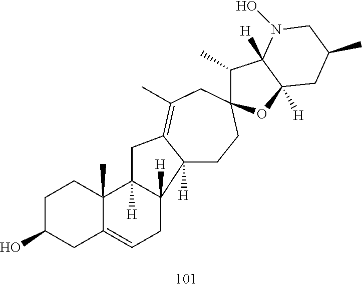

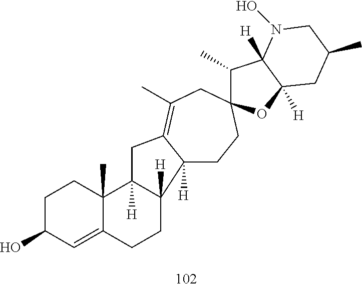

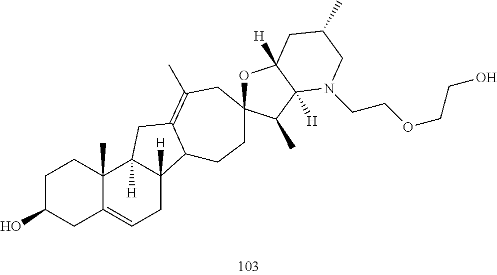

Yet other examples of hedgehog inhibitors include compounds, or pharmaceutically acceptable salts and/or solvates thereof, described in U.S. Patent Application No. 2008/0287420, and also provided below in Table 3:

TABLE 3

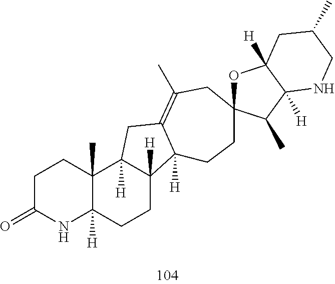

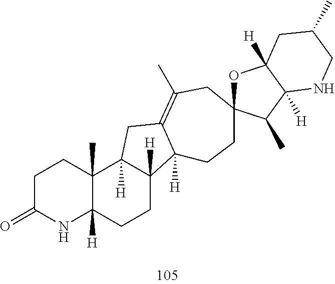

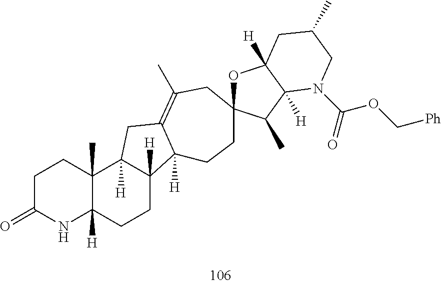

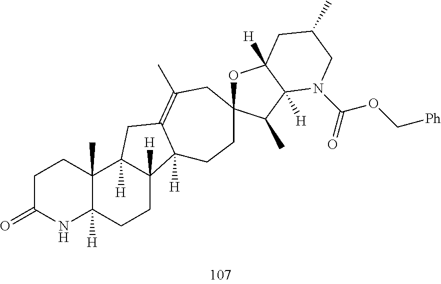

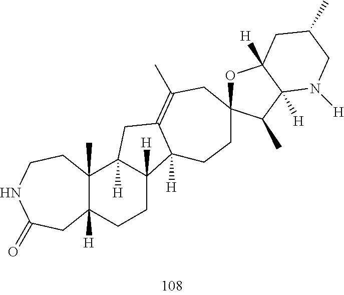

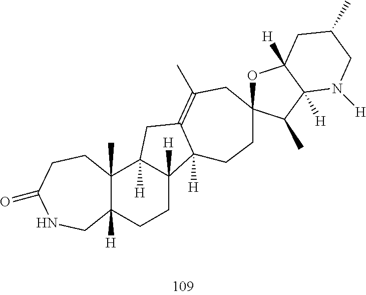

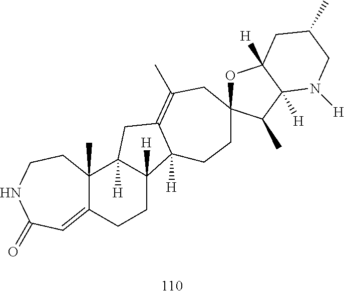

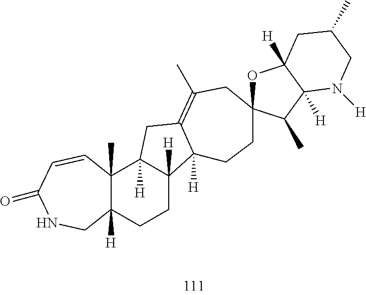

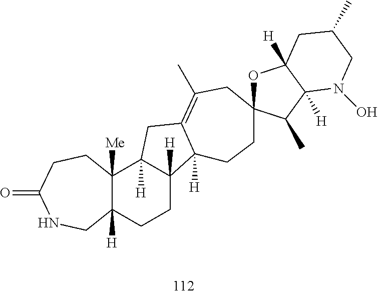

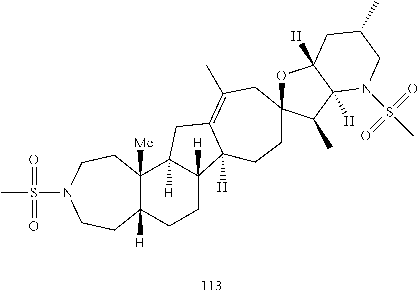

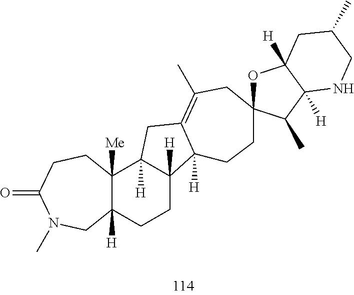

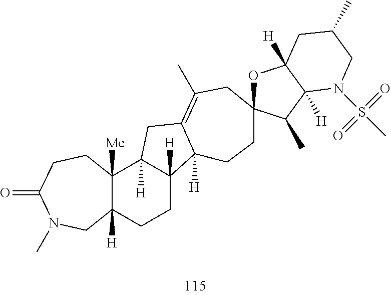

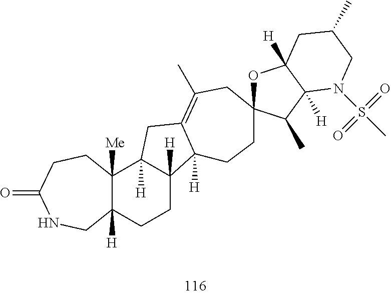

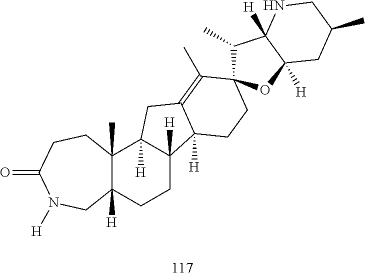

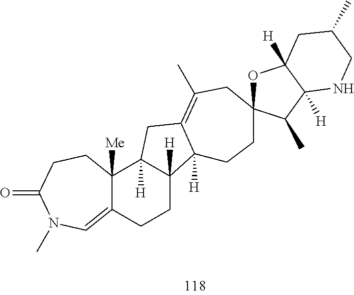

Still yet other examples of hedgehog inhibitors include compounds, or pharmaceutically acceptable salts and/or solvates thereof, described in U.S. Patent Application No. 2008/0293755, and also provided below in Table 4:

TABLE 4

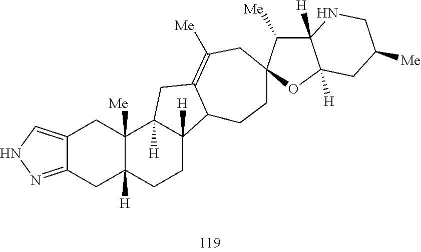

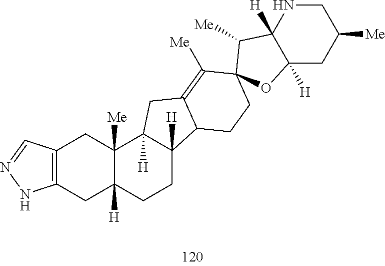

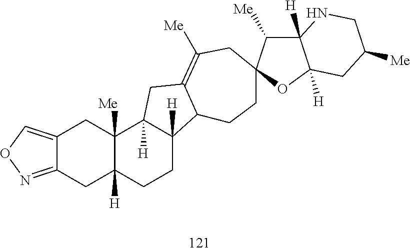

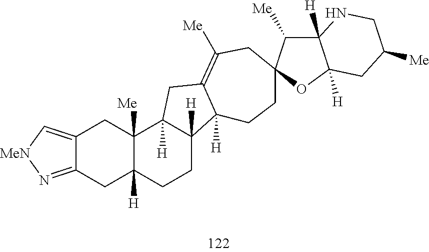

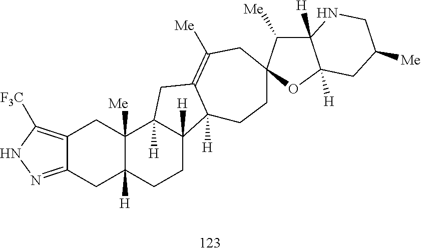

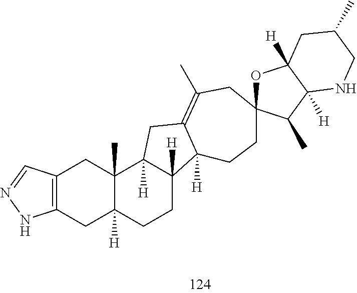

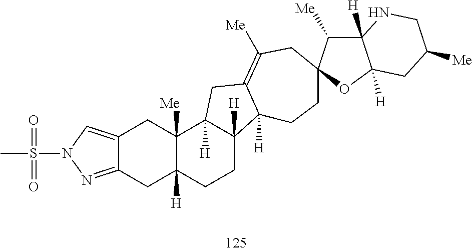

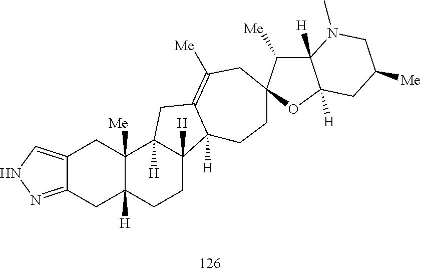

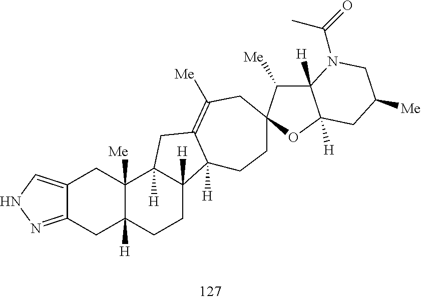

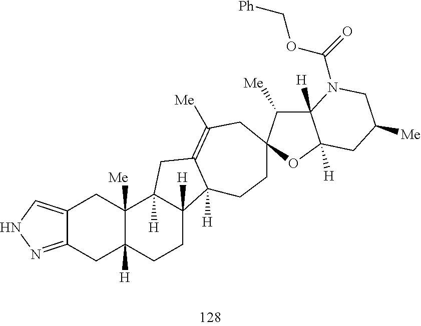

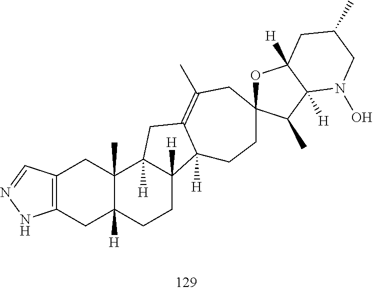

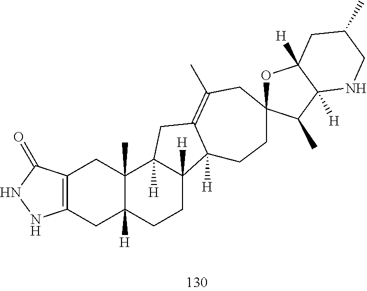

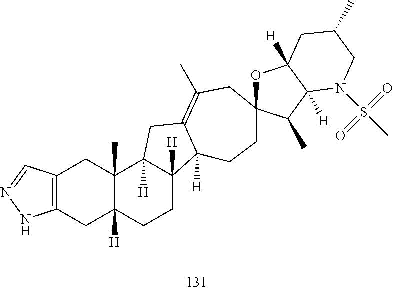

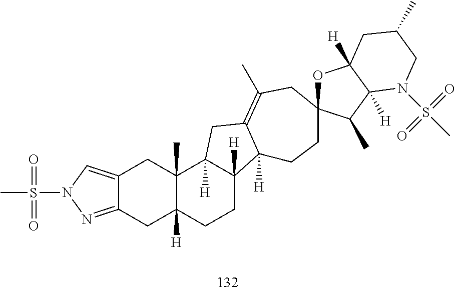

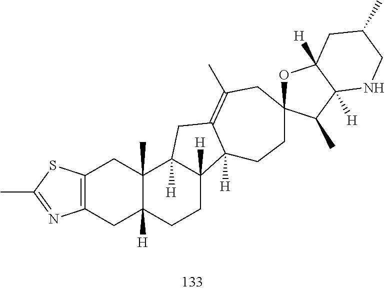

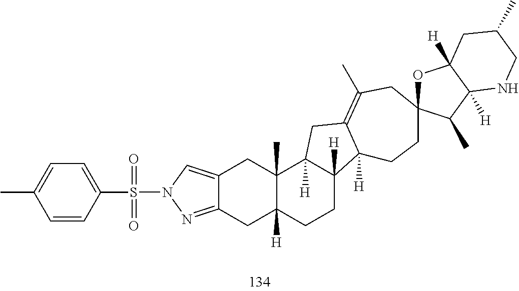

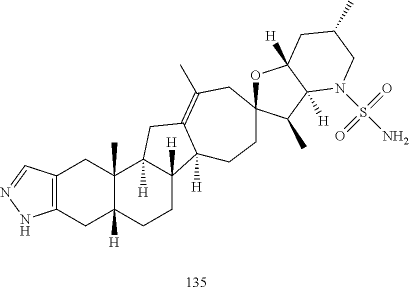

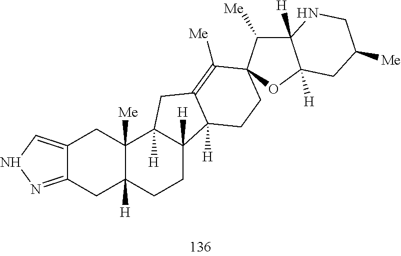

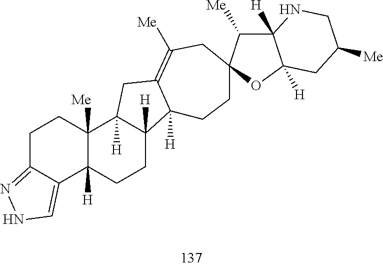

In certain embodiments, the hedgehog inhibitor is the compound 32:

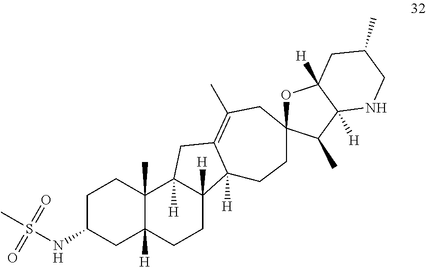

(also referred to herein as IPI-926)

or a pharmaceutically acceptable salt and/or solvate thereof.

Hedgehog inhibitors useful in the current invention can contain a basic functional group, such as amino or alkylamino, and are thus capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately treating the compound in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, besylate, glucoheptonate, lactobionate, and laurylsulphonate salts and the like (see, for example, Berge et al. (1977) “Pharmaceutical Salts”, J. Pharm. Sci. 66:1-19).

Pharmaceutically acceptable salts include, but are not limited to, conventional nontoxic salts or quaternary ammonium salts of the compounds, e.g., from non-toxic organic or inorganic acids. For example, such conventional nontoxic salts include, but are not limited to, those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, benzenesulfonic, ethane disulfonic, oxalic, isothionic, and the like.

In other cases, the compounds can contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately treating the compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

In certain embodiments, the pharmaceutically acceptable salt of IPI-926 is the hydrochloric, hydrobromic, phosphoric, sulfuric, nitric, perchloric, adipic, alginic, ascorbic, aspartic, 2-acetoxybenzoic, benzenesulfonic, benzoic, bisulfonic, boric, butyric, camphoric, camphorsulfonic, citric, cyclopentanepropionic, digluconic, dodecylsulfonic, ethanesulfonic, 1,2-ethanedisulfonic, formic, fumaric, glucoheptonic, glycerophosphonic, gluconic, hemisulfonic, heptanoic, hexanoic, hydroiodic, 2-hydroxyethanesulfonic, hydroxymaleic, isothionic, lactobionic, lactic, lauric, lauryl sulfonic, malic, maleic, malonic, methanesulfonic, 2-naphthalenesulfonic, napthylic, nicotinic, oleic, oxalic, palmitic, pamoic, pectinic, persulfonic, 3-phenylpropionic, picric, pivalic, propionic, phenylacetic, stearic, succinic, salicyclic, sulfanilic, tartaric, thiocyanic, p-toluenesulfonic, undecanoic or valeric acid addition salt.

In certain embodiments, the pharmaceutically acceptable salt of IPI-926 is the hydrochloric acid addition salt.

In certain embodiments, the hedgehog inhibitor is an isopropanol (IPA) solvate of IPI-926 or a pharmaceutically acceptable salt thereof.

Pharmaceutical Compositions

To practice the methods of the invention, the hedgehog inhibitor and/or the therapeutic agent can be delivered in the form of pharmaceutically acceptable compositions which comprise a therapeutically-effective amount of one or more hedgehog inhibitors and/or one or more therapeutic agent formulated together with one or more pharmaceutically acceptable excipients. In some instances, the hedgehog inhibitor and the therapeutic agent are administered in separate pharmaceutical compositions and can (e.g., because of different physical and/or chemical characteristics) be administered by different routes (e.g., one therapeutic is administered orally, while the other is administered intravenously). In other instances, the hedgehog inhibitor and the therapeutic agent can be administered separately, but via the same route (e.g., both orally or both intravenously). In still other instances, the hedgehog inhibitor and the therapeutic agent can be administered in the same pharmaceutical composition.

Pharmaceutical compositions can be specially formulated for administration in solid or liquid form, including those adapted for the following: oral administration, for example, drenches (aqueous or non-aqueous solutions or suspensions), tablets (e.g., those targeted for buccal, sublingual, and systemic absorption), capsules, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin; intravaginally or intrarectally, for example, as a pessary, cream or foam; sublingually; ocularly; transdermally; pulmonarily; or nasally.

Examples of suitable aqueous and nonaqueous carriers which can be employed in pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents, dispersing agents, lubricants, and/or antioxidants. Prevention of the action of microorganisms upon the compounds of the present invention can be ensured by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Methods of preparing these formulations or compositions include the step of bringing into association the hedgehog inhibitor and/or the therapeutic agent with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

The hedgehog inhibitors and the therapeutic agents of the present invention can be given per se or as a pharmaceutical composition containing, for example, about 0.1 to 99%, or about 10 to 50%, or about 10 to 40%, or about 10 to 30%, or about 10 to 20%, or about 10 to 15% of active ingredient in combination with a pharmaceutically acceptable carrier. Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient.

The selected dosage level will depend upon a variety of factors including, for example, the activity of the particular compound employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of a hedgehog inhibitor and/or a therapeutic agent will be that amount of the compound which is the lowest dose effective to produce a therapeutic effect. Such an effective dose will generally depend upon the factors described above. Generally, oral, intravenous and subcutaneous doses of the compounds of the present invention for a patient, when used for the indicated effects, will range from about 0.0001 mg to about 100 mg per day, or about 0.001 mg to about 100 mg per day, or about 0.01 mg to about 100 mg per day, or about 0.1 mg to about 100 mg per day, or about 0.0001 mg to about 500 mg per day, or about 0.001 mg to about 500 mg per day, or about 0.01 mg to about 500 mg per day, or about 0.1 mg to about 500 mg per day.

The subject receiving this treatment is any animal in need, including primates, in particular humans, equines, cattle, swine, sheep, poultry, dogs, cats, mice and rats.

The compounds can be administered daily, every other day, three times a week, twice a week, weekly, or bi-weekly. The dosing schedule can include a “drug holiday,” i.e., the drug can be administered for two weeks on, one week off, or three weeks on, one week off, or four weeks on, one week off, etc., or continuously, without a drug holiday. The compounds can be administered orally, intravenously, intraperitoneally, topically, transdermally, intramuscularly, subcutaneously, intranasally, sublingually, or by any other route.

Since the hedgehog inhibitors are administered in combination with other treatments (such as additional therapeutic agents, radiation or surgery) the doses of each agent or therapy can be lower than the corresponding dose for single-agent therapy. The dose for single-agent therapy can range from, for example, about 0.0001 to about 200 mg, or about 0.001 to about 100 mg, or about 0.01 to about 100 mg, or about 0.1 to about 100 mg, or about 1 to about 50 mg per kilogram of body weight per day. The determination of the mode of administration and the correct dosage is well within the knowledge of the skilled clinician.

### Methods of Treatment

Provided herein are methods of treating a proliferative disorder, such as cancer, comprising orally administering a formulation, as described above and herein, to a patient in need thereof.

A patient to which administration is contemplated includes, but is not limited to, humans (e.g., male, female, infant, child, adolescent, adult, elderly, etc.) and/or other primates; mammals, including commercially relevant mammals such as cattle, pigs, horses, sheep, cats, and/or dogs; and/or birds, including commercially relevant birds such as chickens, ducks, geese, and/or turkeys.

“Treating,” as used herein, refers to administering the minimal amount or concentration of a hedgehog inhibitor, e.g., IPI-926 or a compound of formula (I) or salt thereof that, when administered, confers a therapeutic effect (e.g., controls, relieves, ameliorates, alleviates, or slows the progression of); or prevents (e.g., delays the onset of or reduces the risk of developing) a disease, disorder, or condition or symptoms thereof on the treated subject. In some implementations of the subject matter described herein, treating confers a therapeutic effect (e.g., controls, relieves, ameliorates, alleviates, or slows the progression of) a disease, disorder, or condition or symptoms thereof on the treated subject. In other implementations of the subject matter described herein, treating prevents (e.g., delays the onset of or reduces the risk of developing).

IPI-926, described in PCT publications WO 2008083252 and WO 2008083248, both of which are incorporated herein by reference, has been shown to inhibit in vitro growth of human cell lines derived from patients with pancreatic cancer, medulloblastoma, lung cancer, multiple myeloma, acute lymphocytic leukemia, myelodysplastic syndrome, non-Hodgkin's type lymphoma, Hodgkin's disease and lymphocytic leukemia.

IPI-926 has also shown tumor growth inhibition in a number of preclinical in vivo models, such as medulloblastoma (Pink et al., “Activity of IPI-926, a potent HH pathway inhibitor, in a novel model of medulloblastoma derived from Ptch/HIC+/− mice” American Association for Cancer Research, 1588, 2008; Villavicencia et al., “Activity of the Hh pathway inhibitor IPI-926 in a mouse model of medulloblastoma” American Association for Cancer Research, 2009); small cell lung cancer (Travaglione et al., “A novel Hh pathway inhibitor, IPI-926, delays recurrence post-chemotherapy in a primary human SCLC xenograft model,” American Association for Cancer Research, 4611, 2008; Peacock et al., Visualization of smoothened activation supports an essential role for hedgehog signaling in the regulation of self-renewal in small cell lung cancer, American Association for Cancer Research, 2009); non-small cell lung cancer (Mandley, E., et al. The Hh inhibitor IPI-926 delays tumor re-growth of a non-small cell lung cancer xenograft model following treatment with an EGFR targeted tyrosine kinase inhibitor. American Association for Cancer Research, 2010), skin cancer, head and neck cancer, and ovarian cancer (Growdon et al, “Hedgehog pathway inhibitor cyclopamine suppresses Gli1 expression and inhibits serous ovarian cancer xenograft growth.” Society of Gynecologic Oncologists Annual Meeting on Women's Cancer, 2009).

Additionally, hedgehog inhibitors, e.g., IPI-926, have demonstrated rapid and sustained Hedgehog pathway inhibition in stromal cells, a downstream mediator of Hedgehog signaling, after single administration in a model of human pancreatic cancer (Traviglione et al., EORTC-NCI-AACR Symposium on “Molecular Targets and Cancer Therapeutics” 2008).

Inhibition of the hedgehog pathway has also been shown to reduce or inhibit the growth of a variety of cancers, such as acute lymphocytic leukemia (ALL) (Ji et al., Journal of Biological Chemistry (2007) 282:37370-37377); basal cell carcinoma (Xie et al., Nature (1998) 391:90-92; Williams et al., PNAS (2003) 100:4616-4621; Bale and Yu (2001) Human Molecular Genetics (2001) 10:757-762); biliary cancer (Berman et al., Nature (2003) 425:846-851; WO 2005/013800); brain cancer and glioma (Clement et al., Current Biology (2007) 17:1-8; Ehtesham et al., Ongogene (2007) 1-10); bladder cancer; breast cancer (Kubo et al., Cancer Research (2004) 64:6071-6074; Lewis et al., J. Mammary Gland Biology and Neoplasia (2004) 2:165-181); chondrosarcoma (Wunder et al., Lancet Oncology (2007) 513-524); chronic lymphocytic leukemia (CLL) (Hedge et al., Mol. Cancer Res. (2008) 6:1928-1936); chronic myeloid leukemia (CML) (Dierks et al., Cancer Cell (2008) 14:238-249); colon cancer (Yang and Hinds, BMC Developmental Biology (2007) 7:6); esophageal cancer (Berman et al., Nature (2003) 425:846-851; WO 2005/013800); gastric cancer (Berman et al., Nature (2003) 425:846-851; Ma et al., Carcinogenesis (2005) 26:1698-1705; WO 2005/013800; Shiotani et al., J. Gastroenterol. Hepatol. (2008) S161-S166; Ohta et al., Cancer Research (2005) 65:10822-10829; Ma et al., World J. Gastroenterol (2006) 12:3965-3969); gastrointestinal stromal tumor (GIST) (Yoshizaki et al., World J. Gastroenterol (2006) 12:5687-5691); hepatocellular cancer (Sicklick et al., Carcinogenesis (2006) 27:748-757; Patil et al., Cancer Biology & Therapy (2006) 5:111-117); kidney cancer (Cutcliffe et al., Human Cancer Biology (2005) 11:7986-7994); lung cancer (Watkins et al., Nature (2003) 422:313-317); medulloblastoma (Berman et al., Science (2002) 297:1559-1561; Pietsch et al. Cancer Research (1997) 57:2085-2088); melanoma (Stecca et al., PNAS (2007) 104:5895-5900; Geng et al., Angiogenesis (2007) 10:259-267); multiple myeloma (Peacock et al., PNAS USA (2007) 104:4048-4053; Dierks et al., Nature Medicine (2007) 13:944-951); neuroectodermal tumors (Reifenberger et al., Cancer Research (1998) 58:1798-1803); non-Hodgkin's type lymphoma (NHL) (Dierks et al., Nature Medicine (2007) 13:944-951; Lindemann, Cancer Research (2008) 68:961-964); osteosarcoma (Warzecha et al., J. Chemother. (2007) 19:554-561); ovarian cancer (Steg et al., J. Molecular Diagnostics (2006) 8:76-83); pancreatic cancer (Thayer et al., Nature (2003) 425:851-856; Berman et al., Nature (2003) 425:846-851; WO 2005/013800); prostate cancer (Karhadkar et al., Nature (2004) 431:707-712; Sheng et al., Molecular Cancer (2004) 3:29-42; Fan et al., Endocrinology (2004) 145:3961-3970); and testicular cancer (Dormeyer et al., J. Proteome Res. (2008) 7:2936-2951).

In one aspect, the invention relates to a method of treating cancer by administering to a patient a first therapeutic agent and a second therapeutic agent, wherein the second therapeutic agent is a hedgehog inhibitor. The two agents can be administered concurrently (i.e., essentially at the same time, or within the same treatment) or sequentially (i.e., one immediately following the other, or alternatively, with a gap in between administration of the two). In some embodiments, the hedgehog inhibitor is administered sequentially (i.e., after the first therapeutic). The first therapeutic agent can be a single therapeutic agent, or multiple therapeutic agents administered sequentially or in combination.

In another aspect, the invention relates to a method of treating cancer including the steps of administering to a patient a first therapeutic agent, then administering the first therapeutic agent in combination with a second therapeutic agent, wherein the second therapeutic agent is a hedgehog inhibitor.

In another aspect, the invention relates to a method of treating a condition mediated by the hedgehog pathway by administering to a patient a first therapeutic agent and a second therapeutic agent, wherein the second therapeutic agent is a hedgehog inhibitor. The two agents can be administered concurrently (i.e., essentially at the same time, or within the same treatment) or sequentially (i.e., one immediately following the other, or alternatively, with a gap in between administration of the two). In some embodiments, the hedgehog inhibitor is administered sequentially (i.e., after the first therapeutic). The first therapeutic agent can be a therapeutic agent. In another aspect, the invention relates to a method of treating a condition mediated by the hedgehog pathway including the steps of administering to a patient a first therapeutic agent, then administering the first therapeutic agent in combination with a second therapeutic agent, wherein the second therapeutic agent is a hedgehog inhibitor.

The invention also relates to methods of extending relapse free survival in a cancer patient who is undergoing or has undergone cancer therapy (for example, treatment with one or more therapeutic agents, radiation and/or surgery) by administering a therapeutically effective amount of a hedgehog inhibitor to the patient. “Relapse free survival”, as understood by those skilled in the art, is the length of time following a specific point of cancer treatment during which there is no clinically-defined relapse in the cancer. In some embodiments, the hedgehog inhibitor is administered concurrently with the cancer therapy. In instances of concurrent administration, the hedgehog inhibitor can continue to be administered after the cancer therapy has ceased. In other embodiments, the hedgehog inhibitor is administered after cancer therapy has ceased (i.e., with no period of overlap with the cancer treatment). The hedgehog inhibitor can be administered immediately after cancer therapy has ceased, or there can be a gap in time (e.g., up to about a day, a week, a month, six months, or a year) between the end of cancer therapy and the administration of the hedgehog inhibitor. Treatment with the hedgehog inhibitor can continue for as long as relapse-free survival is maintained (e.g., up to about a day, a week, a month, six months, a year, two years, three years, four years, five years, or longer).

In one aspect, the invention relates to a method of extending relapse free survival in a cancer patient who had previously undergone cancer therapy (for example, treatment with one or more therapeutic agents, radiation and/or surgery) by administering a therapeutically effective amount of a hedgehog inhibitor to the patient after the cancer therapy has ceased. The hedgehog inhibitor can be administered immediately after cancer therapy has ceased, or there can be a gap in time (e.g., up to about a day, a week, a month, six months, or a year) between the end of cancer therapy and the administration of the hedgehog inhibitor.

In some embodiments, the hedgehog inhibitor is a first line treatment for the cancer, i.e., it is used in a subject who has not been previously administered another drug intended to treat the cancer.

In other embodiments, the hedgehog inhibitor is a second line treatment for the cancer, i.e., it is used in a subject who has been previously administered another drug intended to treat the cancer.

In other embodiments, the hedgehog inhibitor is a third or fourth line treatment for the cancer, i.e., it is used in a subject who has been previously administered two or three other drugs intended to treat the cancer.

In some embodiments, a hedgehog inhibitor is administered to a subject following surgical excision/removal of the cancer.

In some embodiments, a hedgehog inhibitor is administered to a subject before, during, and/or after radiation treatment of the cancer.

Exemplary cancers include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer, esophageal cancer (e.g., adenocarcinoma of the esophagus, Barrett's adenocarcinoma), Ewing sarcoma, familiar hypereosinophilia, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphocytic leukemia (ALL), acute myelocytic leukemia (AML), chronic myelocytic leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), leiomyosarcoma (LMS), mastocytosis (e.g., systemic mastocytosis), multiple myeloma (MM), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), Paget's disease of the vulva, Paget's disease of the penis, papillary adenocarcinoma, pancreatic cancer (e.g., pancreatic adenocarcinoma, intraductal papillary mucinous neoplasm (IPMN)), pinealoma, primitive neuroectodermal tumor (PNT), prostate cancer (e.g., prostate adenocarcinoma), rhabdomyosarcoma, retinoblastoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), and Waldenström's macroglobulinemia.

In certain embodiments, the cancer is selected from biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), cervical cancer (e.g., cervical adenocarcinoma), colorectal cancer (e.g., colon cancer, rectal cancer, colorectal adenocarcinoma), gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC)), kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leukemia (e.g., acute lymphocytic leukemia (ALL), acute myelocytic leukemia (AML), chronic myelocytic leukemia (CML), chronic lymphocytic leukemia (CLL)), lymphoma (e.g., Hodgkin lymphoma (HL), non-Hodgkin lymphoma (NHL), follicular lymphoma, diffuse large B-cell lymphoma (DLBCL), mantle cell lymphoma (MCL)), multiple myeloma (MM), myelodysplastic syndrome (MDS), myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), pancreatic cancer (e.g., pancreatic adenocarcinoma, intraductal papillary mucinous neoplasm (IPMN)), prostate cancer (e.g., prostate adenocarcinoma), skin cancer (e.g., squamous cell carcinoma (SCC), keratoacanthoma (KA), melanoma, basal cell carcinoma (BCC)) and soft tissue sarcoma (e.g., malignant fibrous histiocytoma (MFH), liposarcoma, malignant peripheral nerve sheath tumor (MPNST), chondrosarcoma, fibrosarcoma, myxosarcoma).

In certain embodiments, the cancer is selected from bladder cancer, breast cancer, medulloblastoma, colorectal cancer, head and neck cancer, small cell lung cancer (SCLC), non-small cell lung cancer (NSCLC), acute lymphocytic leukemia (ALL), acute myelocytic leukemia (AML), chronic myelocytic leukemia (CML), chronic lymphocytic leukemia (CLL), Hodgkin lymphoma (HL), non-Hodgkin lymphoma (NHL), multiple myeloma (MM), osteosarcoma, ovarian cancer, pancreatic cancer, prostate cancer, basal cell carcinoma (BCC)) and chondrosarcoma.

In certain embodiments, the cancer is bladder cancer.

In certain embodiments, the cancer is breast cancer.

In certain embodiments, the cancer is medulloblastoma.

In certain embodiments, the cancer is an ovarian cancer, e.g., a platinum-resistant ovarian cancer or serous ovarian cancer.

In certain embodiments, the cancer is colorectal cancer.

In certain embodiments, the cancer is head and neck cancer.

In certain embodiments, the cancer is lung cancer. In certain embodiments, the cancer is small cell lung cancer (SCLC). In certain embodiments, the cancer is non-small cell lung cancer (NSCLC).

In certain embodiments, the cancer is leukemia. In certain embodiments, the cancer is acute lymphocytic leukemia (ALL). In certain embodiments, the cancer is acute myelocytic leukemia (AML). In certain embodiments, the cancer is chronic myelocytic leukemia (CML). In certain embodiments, the cancer is chronic lymphocytic leukemia (CLL).

In certain embodiments, the cancer is lymphoma. In certain embodiments, the cancer is Hodgkin lymphoma (HL). In certain embodiments, the cancer is non-Hodgkin lymphoma (NHL).

In certain embodiments, the cancer is multiple myeloma (MM).

In certain embodiments, the cancer is osteosarcoma.

In certain embodiments, the cancer is ovarian cancer.

In certain embodiments, the cancer is pancreatic cancer.

In certain embodiments, the cancer is prostate cancer.

In certain embodiments, the cancer is basal cell carcinoma (BCC).

In certain embodiments, the cancer is a medulloblastoma.

In certain embodiments, the cancer is chondrosarcoma.

In certain embodiments, the cancer is neuroendocrine cancer.

Neuroendocrine cancers (also known as gastroenteropancreatic tumors or gastroenteropancreatic neuroendocrine cancers), are cancers derived from cells at the interface between the endocrine (hormonal) system and the nervous system. The majority of neuroendocrine cancers fall into two categories: carcinoids and pancreatic endocrine tumors (also known as endocrine pancreatic tumors or islet cell tumors). In addition to the two main categories, other forms of neuroendocrine cancers exist, including neuroendocrine lung tumors, which arise from the respiratory rather than the gastro-entero-pancreatic system. Neuroendocrine cancers can originate from endocrine glands such as the adrenal medulla, the pituitary, and the parathyroids, as well as endocrine islets within the thyroid or the pancreas, and dispersed endocrine cells in the respiratory and gastrointestinal tract.

For example, the cancer treated can be a neuroendocrine cancer chosen from one or more of, e.g., a neuroendocrine cancer of the pancreas, lung, appendix, duodenum, ileum, rectum or small intestine. In other embodiments, the neuroendocrine cancer is chosen from one or more of: a pancreatic endocrine tumor; a neuroendocrine lung tumor; or a neuroendocrine cancer from the adrenal medulla, the pituitary, the parathyroids, thyroid endocrine islets, pancreatic endocrine islets, or dispersed endocrine cells in the respiratory or gastrointestinal tract.

Pancreatic endocrine tumors can secrete biologically active peptides (e.g., hormones) that can cause various symptoms in a subject. Such tumors are referred to functional or secretory tumors. Functional tumors can be classified by the hormone most strongly secreted. Examples of functional pancreatic endocrine tumors include gastrinoma (producing excessive gastrin and causing Zollinger-Ellison Syndrome), insulinoma (producing excessive insulin), glucagonoma (producing excessive glucagon), vasoactive intestinal peptideoma (VIPoma, producing excessive vasoactive intestinal peptide), PPoma (producing excessive pancreatic polypeptide), somatostatinoma (producing excessive somatostatin), watery diarrhea hypokalemia-achlorhydria (WDHA), CRHoma (producing excessive corticotropin-releasing hormones), calcitoninoma (producing excessive calcitonin), GHRHoma (producing excessive growth-hormone-releasing hormone), neurotensinoma (producing excessive neurotensin), ACTHoma (producing excessive adrenocorticotropic hormone), GRFoma (producing excessive growth hormone-releasing factor), and parathyroid hormone-related peptide tumor. In some instances, pancreatic endocrine tumors can arise in subjects who have multiple endocrine neoplasia type 1 (MEN1); such tumors often occur in the pituitary gland or pancreatic islet cells. Pancreatic endocrine tumors that do not secrete peptides (e.g., hormones) are called nonfunctional (or nonsecretory or nonfunctional) tumors.

In other embodiments, the cancer treated is a carcinoid tumor, e.g., a carcinoid neuroendocrine cancer. Carcinoid tumors tend to grow more slowly than pancreatic endocrine tumors. A carcinoid tumor can produce biologically active molecules such as serotonin, a biogenic molecule that causes a specific set of symptoms called carcinoid syndrome. Carcinoid tumors that produce biologically active molecules are often referred to as functional carcinoid tumors, while those that do not are referred to as nonfunctional carcinoid tumors. In some embodiments, the neuroendocrine cancer is a functional carcinoid tumor (e.g., a carcinoid tumor that can produce biologically active molecules such as serotonin). In other embodiments, the neuroendocrine cancer is a non-functional carcinoid tumor. In certain embodiments, the carcinoid tumor is a tumor from the thymus, stomach, small intestine (duodenum, jejunum, ileum), large intestine (cecum, colon), rectal, pancreatic, appendix, ovarian or testicular carcinoid.

Carcinoid tumors can be further classified depending on the point of origin, such as lung, thymus, stomach, small intestine (duodenum, jejunum, ileum), large intestine (cecum, colon), rectum, pancreas, appendix, ovaries and testes. In some embodiments, the neuroendocrine cancer is a carcinoid tumor. In other embodiments, the neuroendocrine cancer is a pancreatic endocrine tumor. In still other embodiments, the neuroendocrine cancer is a neuroendocrine lung tumor. In certain embodiments, the neuroendocrine cancers originate from the adrenal medulla, the pituitary, the parathyroids, thyroid endocrine islets, pancreatic endocrine islets, or dispersed endocrine cells in the respiratory or gastrointestinal tract.

Further examples of neuroendocrine cancers that can be treated include, but are not limited to, medullary carcinoma of the thyroid, Merkel cell cancer (trabecular cancer), small-cell lung cancer (SCLC), large-cell neuroendocrine carcinoma (of the lung), extrapulmonary small cell carcinomas (ESCC or EPSCC), neuroendocrine carcinoma of the cervix, Multiple Endocrine Neoplasia type 1 (MEN-1 or MEN1), Multiple Endocrine Neoplasia type 2 (MEN-2 or MEN2), neurofibromatosis type 1, tuberous sclerosis, von Hippel-Lindau (VHL) disease, neuroblastoma, pheochromocytoma (pheochromocytoma), paraganglioma, neuroendocrine cancer of the anterior pituitary, and/or Carney's complex.

In certain embodiments, the cancer has a fibrotic component. In one embodiment, the cancer has fibrosis of the bone marrow or a hematopoietic tissue. In certain embodiments, the fibrotic condition of the bone marrow is an intrinsic feature of a chronic myeloproliferative neoplasm of the bone marrow, such as primary myelofibrosis (also referred to herein as agnogenic myeloid metaplasia or chronic idiopathic myelofibrosis). In other embodiments, the bone marrow fibrosis is associated with (e.g., is secondary to) a malignant condition or a condition caused by a clonal proliferative disease. In other embodiments, the bone marrow fibrosis is associated with a hematologic disorder (e.g., a hematologic disorder chosen from one or more of polycythemia vera, essential thrombocythemia, myelodysplasia, hairy cell leukemia, lymphoma (e.g., Hodgkin or non-Hodgkin lymphoma), multiple myeloma or chronic myelogenous leukemia (CML)). In yet other embodiments, the bone marrow fibrosis is associated with (e.g., secondary to) a non-hematologic disorder (e.g., a non-hematologic disorder chosen from solid tumor metastasis to bone marrow, an autoimmune disorder (e.g., systemic lupus erythematosus, scleroderma, mixed connective tissue disorder, or polymyositis), an infection (e.g., tuberculosis), or secondary hyperparathyroidism associated with vitamin D deficiency.

In embodiments where a fibrotic condition of the bone marrow is treated, the hedgehog inhibitor can be administered in combination with an agent chosen from a Jak2 inhibitor (including, but not limited to, INCB018424, XL019, TG101348, or TG101209), an immunomodulator, e.g., an IMID (including, but not limited to thalidomide, lenalidomide, or panolinomide), hydroxyurea, an androgen, erythropoietic stimulating agents, prednisone, danazol, HDAC inhibitors, or other agents or therapeutic modalities (e.g., stem cell transplants, or radiation).

Certain methods of the current invention can be especially effective in treating cancers that respond well to existing chemotherapies, but suffer from a high relapse rate. In these instances, treatment with the hedgehog inhibitor can increase the relapse-free survival time or rate of the patient. The invention also encompasses the use of a therapeutic agent and a hedgehog inhibitor for preparation of one or more medicaments for use in the methods described herein. The invention also relates to the use of a hedgehog inhibitor in the preparation of a medicament for use in the methods described herein. The invention also encompasses the use of a hedgehog inhibitor in the preparation of a medicament for use in a method of treating a cancer patient as described herein.

Multiple tumor types exhibit up-regulation of Hh ligands post chemotherapy and in response to other stress, such as hypoxia. The type of Hh ligand that is up-regulated (i.e., Sonic, Indian and/or Desert) and the degree of up-regulation vary depending upon the tumor type and the therapeutic agent. Without wishing to be bound to any theory, these results suggest that stress (including chemotherapy) induces Hedgehog ligand production in tumor cells as a protective or survival mechanism. The results further suggest that up-regulation of tumor-derived Hh ligand post-chemotherapy can confer upon the surviving cell population a dependency upon the Hh pathway that is important for tumor recurrence, and thus can be susceptible to Hh pathway inhibition.

Thus, an aspect of the invention is a method of treating cancer by determining whether expression of one or more hedgehog ligands has increased during or after chemotherapy, then administering a hedgehog inhibitor. Ligand expression can be measured by detection of a soluble form of the ligand in peripheral blood and/or urine (e.g., by an ELISA assay or radioimmunoassay), in circulating tumor cells (e.g., by a fluorescence-activated cell sorting (FACS) assay, an immunohistochemistry assay, or a reverse transcription polymerase chain reaction (RT-PCR) assay), or in tumor or bone marrow biopsies (e.g., by an immunohistochemistry assay, a RT-PCR assay, or by in situ hybridization). Detection of hedgehog ligand in a given patient tumor could also be assessed in vivo, by systemic administration of a labeled form of an antibody to a hedgehog ligand followed by imaging, similar to detection of PSMA in prostate cancer patients (Bander, N H Nat Clin Pract Urol 2006; 3:216-225). Expression levels in a patient can be measured at least at two time-points to determine of ligand induction has occurred. For example, hedgehog ligand expression can be measured pre- and post-chemotherapy, pre-chemotherapy and at one or more time-points while chemotherapy is ongoing, or at two or more different time-points while chemotherapy is ongoing. If a hedgehog ligand is found to be up-regulated, a hedgehog inhibitor can be administered. Thus, measurement of hedgehog ligand induction in the patient can determine whether the patient receives a hedgehog pathway inhibitor in combination with or following other chemotherapy.

Another aspect of the invention relates to a method of treating cancer in a patient by identifying one or more therapeutic agents that elevate hedgehog ligand expression in the cancer tumor, and administering one or more of the therapeutic agents that elevate hedgehog ligand expression and a hedgehog inhibitor. To determine which therapeutic agents elevate hedgehog expression, tumor cells can be removed from a patient prior to therapy and exposed to a panel of therapeutic agents ex vivo and assayed to measure changes in hedgehog ligand expression (see, e.g., Am. J. Obstet. Gynecol. November 2003, 189(5):1301-7; J. Neurooncol., February 2004, 66(3):365-75). A therapeutic agent that causes an increase in one or more hedgehog ligands is then administered to the patient. A therapeutic agent that causes an increase in one or more hedgehog ligands can be administered alone or in combination with one or more different therapeutic agents that can or can not cause an increase in one or more hedgehog ligands. The hedgehog inhibitor and therapeutic agent can be administered concurrently (i.e., essentially at the same time, or within the same treatment) or sequentially (i.e., one immediately following the other, or alternatively, with a gap in between administration of the two). Treatment with the hedgehog inhibitor can continue after treatment with the therapeutic agent ceases. Thus, the therapeutic agent is chosen based upon its ability to up-regulate hedgehog ligand expression (which, in turn, renders the tumors dependent upon the hedgehog pathway), which can make the tumor susceptible to treatment with a hedgehog inhibitor.

### Combination Therapy

It will be appreciated that the compositions, e.g., one or more hedgehog inhibitors described herein or pharmaceutical compositions thereof, can be administered in combination with one or more additional therapies, e.g., such as radiation therapy, surgery and/or in combination with one or more therapeutic agents, to treat the cancers described herein.

By “in combination” or “in combination with,” it is not intended to imply that the therapy or the therapeutic agents must be administered at the same time and/or formulated for delivery together, although these methods of delivery are within the scope of the invention. The compositions, e.g., one or more hedgehog inhibitors described herein, can be administered concurrently with, prior to, or subsequent to, a cancer therapy (e.g., a primary cancer therapy, e.g., a cancer therapy that includes one or more other additional therapies or therapeutic agents). In general, each agent will be administered at a dose and/or on a time schedule determined for that agent. In will further be appreciated that the additional therapeutic agent utilized in this combination may be administered together in a single composition or administered separately in different compositions. The particular combination to employ in a regimen will take into account compatibility of the inventive pharmaceutical composition with the additional therapeutically active agent and/or the desired therapeutic effect to be achieved.

In general, it is expected that additional therapeutic agents utilized in combination be utilized at levels that do not exceed the levels at which they are utilized individually. In some embodiments, the levels utilized in combination are expected to be lower than those utilized individually.

In certain embodiments, the hedgehog inhibitor and the additional therapeutic agent are administered concurrently (i.e., administration of the two agents at the same time or day, or within the same treatment regimen) or sequentially (i.e., administration of one agent over a period of time followed by administration of the other agent for a second period of time, or within different treatment regimens).

In certain embodiments, the hedgehog inhibitor and the additional therapeutic agent are administered concurrently. For example, in certain embodiments, the hedgehog inhibitor and the additional therapeutic agent are administered at the same time. In certain embodiments, the hedgehog inhibitor and the additional therapeutic agent are administered on the same day. In certain embodiments, the hedgehog inhibitor is administered after the additional therapeutic agent on the same day or within the same treatment regimen. In certain embodiments, the hedgehog inhibitor is administered before the additional therapeutic agent on the same day or within the same treatment regimen.

In certain embodiments, a hedgehog inhibitor is concurrently administered with additional therapeutic agent for a period of time, after which point treatment with the additional therapeutic agent is stopped and treatment with the hedgehog inhibitor continues.

In other embodiments, a hedgehog inhibitor is concurrently with the additional therapeutic agent for a period of time, after which point treatment with the hedgehog inhibitor is stopped and treatment with the additional therapeutic agent continues.

In certain embodiments, the hedgehog inhibitor and the additional therapeutic agent are administered sequentially. For example, in certain embodiments, the hedgehog inhibitor is administered after the treatment regimen of the additional therapeutic agent has ceased. In certain embodiments, the additional therapeutic agent is administered after the treatment regimen of the hedgehog inhibitor has ceased.

In yet other embodiments, the hedgehog inhibitor, alone or combination with the therapeutic agent is administered in a therapeutically effective amount, e.g., at a predetermined dosage schedule.

In other embodiments, a hedgehog inhibitor and a therapeutic agent can be used in combination with one or more of other therapeutic agents, radiation, and/or surgical procedures.

Cancer therapies include, but are not limited to, surgery and surgical treatments, radiation therapy, and therapeutic agents (e.g., biotherapeutic agents and chemotherapeutic agents).

In certain embodiments, the cancer treated by the methods described herein can be selected from, for example, medulloblastoma, chondrosarcoma, osteosarcoma, pancreatic cancer, lung cancer (e.g., small cell lung cancer (SCLC) or non-small cell lung cancer (NSCLC)), ovarian cancer, head and neck squamous cell carcinoma (HNSCC), chronic myelogenous leukemia (CML), chronic lymphocytic leukemia (CLL), acute lymphoblastic leukemia (ALL), acute myeloid leukemia (AML), multiple myeloma, and prostate cancer.

An example of suitable therapeutics for use in combination with one or more hedgehog inhibitors for treatment of medulloblastoma includes, but is not limited to, a chemotherapeutic agent (e.g., lomustine, cisplatin, carboplatin, vincristine, and cyclophosphamide), radiation therapy, surgery, and a combination thereof.

An example of suitable therapeutics for use in combination with one or more hedgehog inhibitors for treatment of chondrosarcoma includes, but is not limited to, a chemotherapeutic agent (e.g., trabectedin), radiation therapy (e.g., proton therapy), surgery, and a combination thereof.

An example of suitable therapeutics for use in combination with one or more hedgehog inhibitors for treatment of osteosarcoma includes, but is not limited to, a chemotherapeutic agent (e.g., methotrexate (e.g., alone or in combination with leucovorin rescue), cisplatin, adriamycin, ifosfamide (e.g., alone or in combination with mesna), BCG (Bacillus Calmette-Guerin), etoposide, muramyl tri-peptite (MTP)), radiation therapy, surgery, and a combination thereof.

An example of suitable therapeutics for use in combination with one or more hedgehog inhibitors for treatment of pancreatic cancer includes, but is not limited to, a chemotherapeutic agent, e.g., paclitaxel or a paclitaxel agent (e.g., a paclitaxel formulation such as TAXOL®, an albumin-stabilized nanoparticle paclitaxel formulation (e.g., ABRAXANE®) or a liposomal paclitaxel formulation); gemcitabine (e.g., gemcitabine alone or in combination with AXP107-11); other chemotherapeutic agents such as oxaliplatin, 5-fluorouracil, capecitabine, rubitecan, epirubicin hydrochloride, NC-6004, cisplatin, docetaxel (e.g., TAXOTERE®), mitomycin C, ifosfamide; interferon; tyrosine kinase inhibitor (e.g., EGFR inhibitor (e.g., erlotinib, panitumumab, cetuximab, nimotuzumab); HER2/neu receptor inhibitor (e.g., trastuzumab); dual kinase inhibitor (e.g., bosutinib, saracatinib, lapatinib, vandetanib); multikinase inhibitor (e.g., sorafenib, sunitinib, XL184, pazopanib); VEGF inhibitor (e.g., bevacizumab, AV-951, brivanib); radioimmunotherapy (e.g., XR303); cancer vaccine (e.g., GVAX, survivin peptide); COX-2 inhibitor (e.g., celecoxib); IGF-1 receptor inhibitor (e.g., AMG 479, MK-0646); mTOR inhibitor (e.g., everolimus, temsirolimus); IL-6 inhibitor (e.g., CNTO 328); cyclin-dependent kinase inhibitor (e.g., P276-00, UCN-01); Altered Energy Metabolism-Directed (AEMD) compound (e.g., CPI-613); HDAC inhibitor (e.g., vorinostat); TRAIL receptor 2 (TR-2) agonist (e.g., conatumumab); MEK inhibitor (e.g., AS703026, selumetinib, GSK1120212); Raf/MEK dual kinase inhibitor (e.g., RO5126766); Notch signaling inhibitor (e.g., MK0752); monoclonal antibody-antibody fusion protein (e.g., L19IL2); curcumin; HSP90 inhibitor (e.g., IPI-493, IPI-504, tanespimycin, STA-9090); rIL-2; denileukin diftitox; topoisomerase 1 inhibitor (e.g., irinotecan, PEP02); statin (e.g., simvastatin); Factor VIIa inhibitor (e.g., PCI-27483); AKT inhibitor (e.g., RX-0201); hypoxia-activated prodrug (e.g., TH-302); metformin hydrochloride, gamma-secretase inhibitor (e.g., RO4929097); ribonucleotide reductase inhibitor (e.g., 3-AP); immunotoxin (e.g., HuC242-DM4); PARP inhibitor (e.g., KU-0059436, veliparib); CTLA-4 inhibitor (e.g., CP-675,206, ipilimumab); AdV-tk therapy; proteasome inhibitor (e.g., bortezomib (Velcade), NPI-0052); thiazolidinedione (e.g., pioglitazone); NPC-1C; Aurora kinase inhibitor (e.g., R763/AS703569), CTGF inhibitor (e.g., FG-3019); siG12D LODER; and radiation therapy (e.g., tomotherapy, stereotactic radiation, proton therapy), surgery, and a combination thereof. In certain embodiments, a combination of paclitaxel or a paclitaxel agent, and gemcitabine can be used with the pharmaceutical compositions of the invention.

An example of suitable therapeutics for use in combination with one or more hedgehog inhibitors for treatment of small cell lung cancer includes, but is not limited to, a chemotherapeutic agent, e.g., etoposide, carboplatin, cisplatin, irinotecan, topotecan, gemcitabine, liposomal SN-38, bendamustine, temozolomide, belotecan, NK012, FR901228, flavopiridol); tyrosine kinase inhibitor (e.g., EGFR inhibitor (e.g., erlotinib, gefitinib, cetuximab, panitumumab); multikinase inhibitor (e.g., sorafenib, sunitinib); VEGF inhibitor (e.g., bevacizumab, vandetanib); cancer vaccine (e.g., GVAX); Bcl-2 inhibitor (e.g., oblimersen sodium, ABT-263); proteasome inhibitor (e.g., bortezomib (Velcade), NPI-0052), paclitaxel or a paclitaxel agent; docetaxel; IGF-1 receptor inhibitor (e.g., AMG 479); HGF/SF inhibitor (e.g., AMG 102, MK-0646); chloroquine; Aurora kinase inhibitor (e.g., MLN8237); radioimmunotherapy (e.g., TF2); HSP90 inhibitor (e.g., IPI-493, IPI-504, tanespimycin, STA-9090); mTOR inhibitor (e.g., everolimus); Ep-CAM-/CD3-bispecific antibody (e.g., MT110); CK-2 inhibitor (e.g., CX-4945); HDAC inhibitor (e.g., belinostat); SMO antagonist (e.g., BMS 833923); peptide cancer vaccine, and radiation therapy (e.g., intensity-modulated radiation therapy (IMRT), hypofractionated radiotherapy, hypoxia-guided radiotherapy), surgery, and combinations thereof.

An example of suitable therapeutics for use in combination with one or more hedgehog inhibitors for treatment of non-small cell lung cancer includes, but is not limited to, a chemotherapeutic agent, e.g., vinorelbine, cisplatin, docetaxel, pemetrexed disodium, etoposide, gemcitabine, carboplatin, liposomal SN-38, TLK286, temozolomide, topotecan, pemetrexed disodium, azacitidine, irinotecan, tegafur-gimeracil-oteracil potassium, sapacitabine); tyrosine kinase inhibitor (e.g., EGFR inhibitor (e.g., erlotinib, gefitinib, cetuximab, panitumumab, necitumumab, PF-00299804, nimotuzumab, RO5083945), MET inhibitor (e.g., PF-02341066, ARQ 197), PI3K kinase inhibitor (e.g., XL147, GDC-0941), Raf/MEK dual kinase inhibitor (e.g., RO5126766), PI3K/mTOR dual kinase inhibitor (e.g., XL765), SRC inhibitor (e.g., dasatinib), dual inhibitor (e.g., BIBW 2992, GSK1363089, ZD6474, AZD0530, AG-013736, lapatinib, MEHD7945A, linifanib), multikinase inhibitor (e.g., sorafenib, sunitinib, pazopanib, AMG 706, XL184, MGCD265, BMS-690514, R935788), VEGF inhibitor (e.g., endostar, endostatin, bevacizumab, cediranib, BIBF 1120, axitinib, tivozanib, AZD2171), cancer vaccine (e.g., BLP25 liposome vaccine, GVAX, recombinant DNA and adenovirus expressing L523S protein), Bcl-2 inhibitor (e.g., oblimersen sodium), proteasome inhibitor (e.g., bortezomib, carfilzomib, NPI-0052, MLN9708), paclitaxel or a paclitaxel agent, docetaxel, IGF-1 receptor inhibitor (e.g., cixutumumab, MK-0646, OSI 906, CP-751,871, BIIB022), hydroxychloroquine, HSP90 inhibitor (e.g., IPI-493, IPI-504, tanespimycin, STA-9090, AUY922, XL888), mTOR inhibitor (e.g., everolimus, temsirolimus, ridaforolimus), Ep-CAM-/CD3-bispecific antibody (e.g., MT110), CK-2 inhibitor (e.g., CX-4945), HDAC inhibitor (e.g., MS 275, LBH589, vorinostat, valproic acid, FR901228), DHFR inhibitor (e.g., pralatrexate), retinoid (e.g., bexarotene, tretinoin), antibody-drug conjugate (e.g., SGN-15), bisphosphonate (e.g., zoledronic acid), cancer vaccine (e.g., belagenpumatucel-L), low molecular weight heparin (LMWH) (e.g., tinzaparin, enoxaparin), GSK1572932A, melatonin, talactoferrin, dimesna, topoisomerase inhibitor (e.g., amrubicin, etoposide, karenitecin), nelfinavir, cilengitide, ErbB3 inhibitor (e.g., MM-121, U3-1287), survivin inhibitor (e.g., YM155, LY2181308), eribulin mesylate, COX-2 inhibitor (e.g., celecoxib), pegfilgrastim, Polo-like kinase 1 inhibitor (e.g., BI 6727), TRAIL receptor 2 (TR-2) agonist (e.g., CS-1008), CNGRC peptide-TNF alpha conjugate, dichloroacetate (DCA), HGF inhibitor (e.g., SCH 900105), SAR240550, PPAR-gamma agonist (e.g., CS-7017), gamma-secretase inhibitor (e.g., RO4929097), epigenetic therapy (e.g., 5-azacitidine), nitroglycerin, MEK inhibitor (e.g., AZD6244), cyclin-dependent kinase inhibitor (e.g., UCN-01), cholesterol-Fus1, antitubulin agent (e.g., E7389), farnesyl-OH-transferase inhibitor (e.g., lonafarnib), immunotoxin (e.g., BB-10901, SS1 (dsFv) PE38), fondaparinux, vascular-disrupting agent (e.g., AVE8062), PD-L1 inhibitor (e.g., MDX-1105, MDX-1106), beta-glucan, NGR-hTNF, EMD 521873, MEK inhibitor (e.g., GSK1120212), epothilone analog (e.g., ixabepilone), kinesin-spindle inhibitor (e.g., 4SC-205), telomere targeting agent (e.g., KML-001), P70 pathway inhibitor (e.g., LY2584702), AKT inhibitor (e.g., MK-2206), angiogenesis inhibitor (e.g., lenalidomide), Notch signaling inhibitor (e.g., OMP-21M18), radiation therapy, surgery, and combinations thereof.

An example of suitable therapeutics for use in combination with one or more hedgehog inhibitors for treatment of ovarian cancer includes, but is not limited to, a chemotherapeutic agent (e.g., paclitaxel or a paclitaxel agent; docetaxel; carboplatin; gemcitabine; doxorubicin; topotecan; cisplatin; irinotecan, TLK286, ifosfamide, olaparib, oxaliplatin, melphalan, pemetrexed disodium, SJG-136, cyclophosphamide, etoposide, decitabine); ghrelin antagonist (e.g., AEZS-130), immunotherapy (e.g., APC8024, oregovomab, OPT-821), tyrosine kinase inhibitor (e.g., EGFR inhibitor (e.g., erlotinib), dual inhibitor (e.g., E7080), multikinase inhibitor (e.g., AZD0530, JI-101, sorafenib, sunitinib, pazopanib), ON 01910.Na), VEGF inhibitor (e.g., bevacizumab, BIBF 1120, cediranib, AZD2171), PDGFR inhibitor (e.g., IMC-3G3), paclitaxel, topoisomerase inhibitor (e.g., karenitecin, Irinotecan), HDAC inhibitor (e.g., valproate, vorinostat), folate receptor inhibitor (e.g., farletuzumab), angiopoietin inhibitor (e.g., AMG 386), epothilone analog (e.g., ixabepilone), proteasome inhibitor (e.g., carfilzomib), IGF-1 receptor inhibitor (e.g., OSI 906, AMG 479), PARP inhibitor (e.g., veliparib, AG014699, iniparib, MK-4827), Aurora kinase inhibitor (e.g., MLN8237, ENMD-2076), angiogenesis inhibitor (e.g., lenalidomide), DHFR inhibitor (e.g., pralatrexate), radioimmunotherapeutic agent (e.g., Hu3S193), statin (e.g., lovastatin), topoisomerase 1 inhibitor (e.g., NKTR-102), cancer vaccine (e.g., p53 synthetic long peptides vaccine, autologous OC-DC vaccine), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, Polo-like kinase 1 inhibitor (e.g., BI 6727), gamma-secretase inhibitor (e.g., RO4929097), Wee-1 inhibitor (e.g., MK-1775), antitubulin agent (e.g., vinorelbine, E7389), immunotoxin (e.g., denileukin diftitox), SB-485232, vascular-disrupting agent (e.g., AVE8062), integrin inhibitor (e.g., EMD 525797), kinesin-spindle inhibitor (e.g., 4SC-205), revlimid, HER2 inhibitor (e.g., MGAH22), ErrB3 inhibitor (e.g., MM-121), radiation therapy; and combinations thereof.

An example of suitable therapeutics for use in combination with one or more hedgehog inhibitors for treatment of chronic myelogenous leukemia (AML) according to the invention includes, but is not limited to, a chemotherapeutic (e.g., cytarabine (Ara-C), hydroxyurea, clofarabine, melphalan, thiotepa, fludarabine, busulfan, etoposide, cordycepin, pentostatin, capecitabine, azacitidine, cyclophosphamide, cladribine, topotecan), tyrosine kinase inhibitor (e.g., BCR/ABL inhibitor (e.g., imatinib, nilotinib), ON 01910.Na, dual inhibitor (e.g., dasatinib, bosutinib), multikinase inhibitor (e.g., DCC-2036, ponatinib, sorafenib, sunitinib, RGB-286638)), interferon alfa, steroids, apoptotic agent (e.g., omacetaxine mepesuccinat), immunotherapy (e.g., allogeneic CD4+ memory Th1-like T cells/microparticle-bound anti-CD3/anti-CD28, autologous cytokine induced killer cells (CIK), AHN-12), CD52 targeting agent (e.g., alemtuzumab), HSP90 inhibitor (e.g., IPI-493, IPI-504, tanespimycin, STA-9090, AUY922, XL888), mTOR inhibitor (e.g., everolimus), SMO antagonist (e.g., BMS 833923), ribonucleotide reductase inhibitor (e.g., 3-AP), JAK-2 inhibitor (e.g., INCB018424), Hydroxychloroquine, retinoid (e.g., fenretinide), cyclin-dependent kinase inhibitor (e.g., UCN-01), HDAC inhibitor (e.g., belinostat, vorinostat, JNJ-26481585), PARP inhibitor (e.g., veliparib), MDM2 antagonist (e.g., RO5045337), Aurora B kinase inhibitor (e.g., TAK-901), radioimmunotherapy (e.g., actinium-225-labeled anti-CD33 antibody HuM195), Hedgehog inhibitor (e.g., PF-04449913), STATS inhibitor (e.g., OPB-31121), KB004, cancer vaccine (e.g., AG858), bone marrow transplantation, stem cell transplantation, radiation therapy, and combinations thereof. In one embodiment, the AML treatment includes one or more hedgehog inhibitors in combination with high dose Ara-C (HDAC). An exemplary HDAC treatment includes high-dose cytarabine at a dose of 3000 mg/m2 every 12 (q12) hours on days 1, 3 and 5 (total of 6 doses).

An example of suitable therapeutics for use in combination with one or more hedgehog inhibitors for treatment of chronic lymphocytic leukemia (CLL) includes, but is not limited to, a chemotherapeutic agent (e.g., fludarabine, cyclophosphamide, doxorubicin, vincristine, chlorambucil, bendamustine, chlorambucil, busulfan, gemcitabine, melphalan, pentostatin, mitoxantrone, 5-azacytidine, pemetrexed disodium), tyrosine kinase inhibitor (e.g., EGFR inhibitor (e.g., erlotinib), BTK inhibitor (e.g., PCI-32765), multikinase inhibitor (e.g., MGCD265, RGB-286638), CD20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., ABT-263), immunotherapy (e.g., allogeneic CD4+ memory Th1-like T cells/microparticle-bound anti-CD3/anti-CD28, autologous cytokine induced killer cells (CIK)), HDAC inhibitor (e.g., vorinostat, valproic acid, LBH589, JNJ-26481585, AR-42), XIAP inhibitor (e.g., AEG35156), CD-74 targeting agent (e.g., milatuzumab), mTOR inhibitor (e.g., everolimus), AT-101, immunotoxin (e.g., CAT-8015, anti-Tac(Fv)-PE38 (LMB-2)), CD37 targeting agent (e.g., TRU-016), radioimmunotherapy (e.g., 131-tositumomab), hydroxychloroquine, perifosine, SRC inhibitor (e.g., dasatinib), thalidomide, PI3K delta inhibitor (e.g., CAL-101), retinoid (e.g., fenretinide), MDM2 antagonist (e.g., RO5045337), plerixafor, Aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), hypoxia-activated prodrug (e.g., TH-302), paclitaxel or a paclitaxel agent, HSP90 inhibitor, AKT inhibitor (e.g., MK2206), HMG-CoA inhibitor (e.g., simvastatin), GNKG186, radiation therapy, bone marrow transplantation, stem cell transplantation, and a combination thereof.

An example of suitable therapeutics for use in combination with one or more hedgehog inhibitors for treatment of acute lymphocytic leukemia (ALL) includes, but is not limited to, a chemotherapeutic agent (e.g., prednisolone, dexamethasone, vincristine, asparaginase, daunorubicin, cyclophosphamide, cytarabine, etoposide, thioguanine, mercaptopurine, clofarabine, liposomal annamycin, busulfan, etoposide, capecitabine, decitabine, azacitidine, topotecan, temozolomide), tyrosine kinase inhibitor (e.g., BCR/ABL inhibitor (e.g., imatinib, nilotinib), ON 01910.Na, multikinase inhibitor (e.g., sorafenib)), CD-20 targeting agent (e.g., rituximab), CD52 targeting agent (e.g., alemtuzumab), HSP90 inhibitor (e.g., STA-9090), mTOR inhibitor (e.g., everolimus, rapamycin), JAK-2 inhibitor (e.g., INCB018424), HER2/neu receptor inhibitor (e.g., trastuzumab), proteasome inhibitor (e.g., bortezomib), methotrexate, asparaginase, CD-22 targeting agent (e.g., epratuzumab, inotuzumab), immunotherapy (e.g., autologous cytokine induced killer cells (CIK), AHN-12), blinatumomab, cyclin-dependent kinase inhibitor (e.g., UCN-01), CD45 targeting agent (e.g., BC8), MDM2 antagonist (e.g., RO5045337), immunotoxin (e.g., CAT-8015, DT2219ARL), HDAC inhibitor (e.g., JNJ-26481585), JVRS-100, paclitaxel or a paclitaxel agent, STATS inhibitor (e.g., OPB-31121), PARP inhibitor (e.g., veliparib), EZN-2285, radiation therapy, steroid, bone marrow transplantation, stem cell transplantation, or a combination thereof.

An example of suitable therapeutics for use in combination with one or more hedgehog inhibitors for treatment of acute myeloid leukemia (AML) includes, but is not limited to, a chemotherapeutic agent (e.g., cytarabine, daunorubicin, idarubicin, clofarabine, decitabine, vosaroxin, azacitidine, clofarabine, ribavirin, CPX-351, treosulfan, elacytarabine, azacitidine), tyrosine kinase inhibitor (e.g., BCR/ABL inhibitor (e.g., imatinib, nilotinib), ON 01910.Na, multikinase inhibitor (e.g., midostaurin, SU 11248, quizartinib, sorafinib)), immunotoxin (e.g., gemtuzumab ozogamicin), DT388IL3 fusion protein, HDAC inhibitor (e.g., vorinostat, LBH589), plerixafor, mTOR inhibitor (e.g., everolimus), SRC inhibitor (e.g., dasatinib), HSP90 inhibitor (e.g., STA-9090), retinoid (e.g., bexarotene, Aurora kinase inhibitor (e.g., BI 811283), JAK-2 inhibitor (e.g., INCB018424), Polo-like kinase inhibitor (e.g., BI 6727), cenersen, CD45 targeting agent (e.g., BC8), cyclin-dependent kinase inhibitor (e.g., UCN-01), MDM2 antagonist (e.g., RO5045337), mTOR inhibitor (e.g., everolimus), LY573636-sodium, ZRx-101, MLN4924, lenalidomide, immunotherapy (e.g., AHN-12), histamine dihydrochloride, radiation therapy, bone marrow transplantation, stem cell transplantation, and a combination thereof.

An example of suitable therapeutics for use in combination with one or more hedgehog inhibitors for treatment of multiple myeloma (MM) includes, but is not limited to, a chemotherapeutic agent (e.g., melphalan, amifostine, cyclophosphamide, doxorubicin, clofarabine, bendamustine, fludarabine, adriamycin, SyB L-0501), thalidomide, lenalidomide, dexamethasone, prednisone, pomalidomide, proteasome inhibitor (e.g., bortezomib, carfilzomib, MLN9708), cancer vaccine (e.g., GVAX), CD-40 targeting agent (e.g., SGN-40, CHIR-12.12), perifosine, zoledronic acid, Immunotherapy (e.g., MAGE-A3, NY-ESO-1, HuMax-CD38), HDAC inhibitor (e.g., vorinostat, LBH589, AR-42), aplidin, cycline-dependent kinase inhibitor (e.g., PD-0332991, dinaciclib), arsenic trioxide, CB3304, HSP90 inhibitor (e.g., KW-2478), tyrosine kinase inhibitor (e.g., EGFR inhibitor (e.g., cetuximab), multikinase inhibitor (e.g., AT9283)), VEGF inhibitor (e.g., bevacizumab), plerixafor, MEK inhibitor (e.g., AZD6244), IPH2101, atorvastatin, immunotoxin (e.g., BB-10901), NPI-0052, radioimmunotherapeutic (e.g., yttrium Y 90 ibritumomab tiuxetan), STATS inhibitor (e.g., OPB-31121), MLN4924, Aurora kinase inhibitor (e.g., ENMD-2076), IMGN901, ACE-041, CK-2 inhibitor (e.g., CX-4945), radiation therapy, bone marrow transplantation, stem cell transplantation, and a combination thereof.

An example of suitable therapeutics for use in combination with one or more hedgehog inhibitors for treatment of head and neck cancer includes, but is not limited to, a chemotherapeutic (e.g., paclitaxel or a paclitaxel agent, carboplatin, docetaxel, amifostine, cisplatin, oxaliplatin, docetaxel), tyrosine kinase inhibitors (e.g., EGFR inhibitor (e.g., erlotinib, gefitinib, icotinib, cetuximab, panitumumab, zalutumumab, nimotuzumab, necitumumab, matuzumab, cetuximab), dual inhibitor (e.g., lapatinib, neratinib, vandetanib, BIBW 2992, multikinase inhibitor (e.g., XL-647)), VEGF inhibitor (e.g., bevacizumab), reovirus, radiation therapy, surgery, and a combination thereof.

An example of suitable therapeutics for use in combination with one or more hedgehog inhibitors for treatment of prostate cancer includes, but is not limited to, a chemotherapeutic agent (e.g., docetaxel, carboplatin, fludarabine), abiraterone, hormonal therapy (e.g., flutamide, bicalutamide, nilutamide, cyproterone acetate, ketoconazole, aminoglutethimide, abarelix, degarelix, leuprolide, goserelin, triptorelin, buserelin), tyrosine kinase inhibitor (e.g., dual kinase inhibitor (e.g., lapatanib), multikinase inhibitor (e.g., sorafenib, sunitinib)), VEGF inhibitor (e.g., bevacizumab), TAK-700, cancer vaccine (e.g., BPX-101, PEP223), lenalidomide, TOK-001, IGF-1 receptor inhibitor (e.g., cixutumumab), TRC105, Aurora A kinase inhibitor (e.g., MLN8237), proteasome inhibitor (e.g., bortezomib), OGX-011, radioimmunotherapy (e.g., HuJ591-GS), HDAC inhibitor (e.g., valproic acid, SB939, LBH589), hydroxychloroquine, mTOR inhibitor (e.g., everolimus), dovitinib lactate, diindolylmethane, efavirenz, OGX-427, genistein, IMC-3G3, bafetinib, CP-675,206, radiation therapy, surgery, or a combination thereof.

In some embodiments, the one or more hedgehog inhibitors described herein is used in combination with a mTOR inhibitor, e.g., one or more mTOR inhibitors chosen from one or more of rapamycin, temsirolimus (TORISEL®), everolimus (RAD001, AFINITOR®), ridaforolimus, AP23573, AZD8055, BEZ235, BGT226, XL765, PF-4691502, GDC0980, SF1126, OSI-027, GSK1059615, KU-0063794, WYE-354, INK128, temsirolimus (CCI-779), Palomid 529 (P529), PF-04691502, or PKI-587. In one embodiment, the mTOR inhibitor inhibits TORC1 and TORC2. Examples of TORC1 and TORC2 dual inhibitors include, e.g., OSI-027, XL765, Palomid 529, and INK128.

In some embodiments, the one or more hedgehog inhibitors described herein is used in combination with an inhibitor of insulin-like growth factor receptor (IGF-1R), e.g., BMS-536924, GSK1904529A, AMG 479, MK-0646, cixutumumab, OSI 906, figitumumab (CP-751,871), or BIIB022.

In some embodiments, the one or more hedgehog inhibitors described herein is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-B inhibitor)), a RAF-1 inhibitor, a KIT inhibitor and a RET inhibitor. In some embodiments, the anti-cancer agent used in combination with the hedgehog inhibitor is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074, nSorafenib Tosylate (Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In some embodiments, the one or more hedgehog inhibitors described herein is used in combination with folfirinox comprising oxaliplatin 85 mg/m2 and irinotecan 180 mg/m2 plus leucovorin 400 mg/m2 followed by bolus fluorouracil (5-FU) 400 mg/m2 on day 1, then 5-FU 2,400 mg/m2 as a 46-hour continuous infusion.

In some embodiments, the one or more hedgehog inhibitors described herein is used in combination with a PI3K inhibitor. In one embodiment, the PI3K inhibitor is an inhibitor of delta and gamma isoforms of PI3K. Exemplary PI3K inhibitors that can be used in combination are described in, e.g., WO 09/088,990; WO 09/088,086; WO 2011/008302; WO 2010/036380; WO 2010/006086, WO 09/114,870, WO 05/113556; US 2009/0312310, US 2011/0046165. Additional PI3K inhibitors that can be used in combination with the hedgehog inhibitors, include but are not limited to, GSK 2126458, GDC-0980, GDC-0941, Sanofi XL147, XL756, XL147, PF-46915032, BKM 120, CAL-101, CAL 263, SF1126, PX-886, and a dual PI3K inhibitor (e.g., Novartis BEZ235). In one embodiment, the PI3K inhibitor is an isoquinolinone. In one embodiment, the PI3K inhibitor is INK1197 or a derivative thereof. In other embodiments, the PI3K inhibitor is INK1117 or a derivative thereof.

In some embodiments, the one or more hedgehog inhibitors described herein is used in combination with a HSP90 inhibitor. The HSP90 inhibitor can be a geldanamycin derivative, e.g., a benzoquinone or hydroquinone ansamycin HSP90 inhibitor (e.g., IPI-493 and/or IPI-504). Non-limiting examples of HSP90 inhibitors include IPI-493, IPI-504, 17-AAG (also known as tanespimycin or CNF-1010), BIIB-021 (CNF-2024), BIIB-028, AUY-922 (also known as VER-49009), SNX-5422, STA-9090, AT-13387, XL-888, MPC-3100, CU-0305, 17-DMAG, CNF-1010, Macbecin (e.g., Macbecin I, Macbecin II), CCT-018159, CCT-129397, PU-H71, or PF-04928473 (SNX-2112).

In some embodiments, the one or more hedgehog inhibitors described herein is administered in combination with a BRAF inhibitor, e.g., GSK2118436, RG7204, PLX4032, GDC-0879, PLX4720, and sorafenib tosylate (Bay 43-9006).

In some embodiments, the one or more hedgehog inhibitors described herein is administered in combination with a MEK inhibitor, e.g., ARRY-142886, GSK1120212, RDEA436, RDEA119/BAY 869766, AS703026, AZD6244 (selumetinib), BIX 02188, BIX 02189, CI-1040 (PD184352), PD0325901, PD98059, and U0126.

In some embodiments, the one or more hedgehog inhibitors described herein is administered in combination with a JAK2 inhibitor, e.g., CEP-701, INCB18424, CP-690550 (tasocitinib).

In one embodiment, the second agent is a taxane, e.g. paclitaxel or a formulation thereof (e.g., albumin-bound paclitaxel (ABRAXANE®), nab-paclitaxel), docetaxel (e.g., as an injectable Docetaxel (Taxotere®)), or taxol).

In some embodiments, the one or more hedgehog inhibitors described herein is administered in combination with paclitaxel or a paclitaxel agent, e.g., TAXOL®, protein-bound paclitaxel (e.g., ABRAXANE®). A “paclitaxel agent” as used herein refers to a formulation of paclitaxel (e.g., for example, TAXOL®) or a paclitaxel equivalent (e.g., for example, a prodrug of paclitaxel). Exemplary paclitaxel equivalents include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX®, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620). In certain embodiments, the paclitaxel agent is a paclitaxel equivalent. In certain embodiments, the paclitaxel equivalent is ABRAXANE®.

Radiation therapy can be administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner as disclosed herein include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Inhibition of the Hedgehog Pathway

Cancer cell killing by inhibition of a component of the hedgehog pathway can be ascertained using the following assay. C3H10T1/2 cells differentiate into osteoblasts when contacted with the sonic hedgehog peptide (Shh-N). Upon differentiation, these osteoblasts produce high levels of alkaline phosphatase (AP) which can be measured in an enzymatic assay (Nakamura et al., 1997 BBRC 237: 465). Compounds that block the differentiation of C3H10T1/2 into osteoblasts (a Shh dependent event) can therefore be identified by a reduction in AP production (van der Horst et al., 2003 Bone 33: 899). The assay details are described below. Additional assays to ascertain the activity of hedgehog inhibitors, including IPI-926, are described in US 2009/0181997 by Grayzel et al.; U.S. Ser. No. 61/327,373 and 61/331,365, filed on Apr. 23, 2010 and May 4, 2010, respectively; the entire contents of the aforesaid applications are incorporated herein by reference.

### Cell Culture

Mouse embryonic mesoderm fibroblasts C3H10T1/2 cells (obtained from ATCC) were cultured in Basal MEM Media (Gibco/Invitrogen) supplemented with 10% heat inactivated FBS (Hyclone), 50 units/ml penicillin and 50 ug/ml streptomycin (Gibco/Invitrogen) at 37° C. with 5% CO₂ in air atmosphere.

### Alkaline Phosphatase Assay

C3H10T1/2 cells were plated in 96 wells with a density of 8×10³ cells/well. Cells were grown to confluence (72 hrs.). After sonic hedgehog (250 ng/ml) and/or compound treatment, the cells were lysed in 110 μL of lysis buffer (50 mM Tris pH 7.4, 0.1% TritonX100), plates were sonicated and lysates spun through 0.2 μm PVDF plates (Corning). 40 μL of lysates was assayed for AP activity in alkaline buffer solution (Sigma) containing 1 mg/ml p-Nitrophenyl Phosphate. After incubating for 30 min at 37° C., the plates were read on an Envision plate reader at 405 nm. Total protein was quantified with a BCA protein assay kit from Pierce according to manufacturer's instructions. AP activity was normalized against total protein. Using the above-described assay, IPI-926 (HCl salt) was shown to be an antagonist of the hedgehog pathway with an IC₅₀ less than 20 nM.

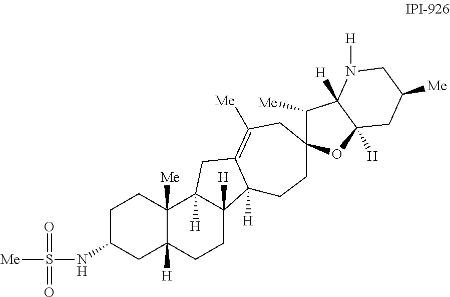

### Example 2

### Improved Efficacy of the Combination of Chemotherapy and Hedgehog Inhibition

This example demonstrates that IPI-926 used as a single agent following cyto-reductive chemotherapy has a growth inhibitory effect on the re-growth of tumors. The data shown herein underscore the importance of continuity between therapeutic agent treatment and subsequent IPI-926 treatment to optimize the inhibitory tumor effects.

IPI-926 was shown to delay primary small cell lung (LX22) tumor recurrence following chemotherapy in xenograft tumor models. Briefly, LX22 primary small cell lung model was treated with 1.5 cycles of etoposide/carboplatin (E/P). Administration of IPI-926 was initiated 24 hours after the last dose of chemotherapy. In the normal course of these studies, IPI-926 was administered on the final day of chemotherapy dosing.

FIG. 1 shows the effect in tumor size as a function of time of treatment of LX22 primary small cell lung model treated with IPI-926 alone (“IPI-926”), etoposide/carboplatin followed by vehicle control (“E/P→Vehicle”), E/P followed by IPI-926 (“E/P→IPI-926”) and vehicle control. IPI-926 used as a single agent following cyto-reductive chemotherapy has an inhibitory effect on the re-growth of tumors.

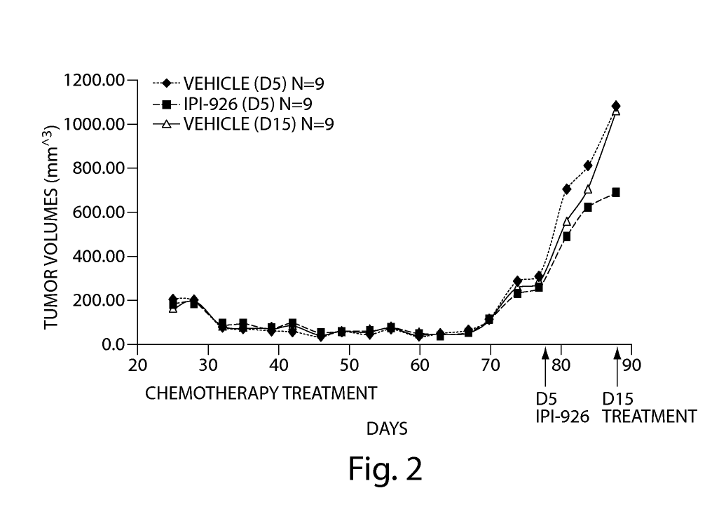

The effect of delaying the onset of IPI-926 administration following chemotherapy was further characterized. Delaying IPI-926 administration by either 5 or 14 days resulted in a loss of the IPI-926 effect in tumor growth inhibition following chemotherapy. FIG. 2 is a linear graph depicting the effect in tumor size as a function of time of chemotherapy treatment and following with IPI-926 treatment on day 5 (D5) and day 15 (D15) following chemotherapy treatment. Thus, a narrow window of intervention is necessary to maximize the beneficial effects of IPI-926 in tumor inhibition following chemotherapy.

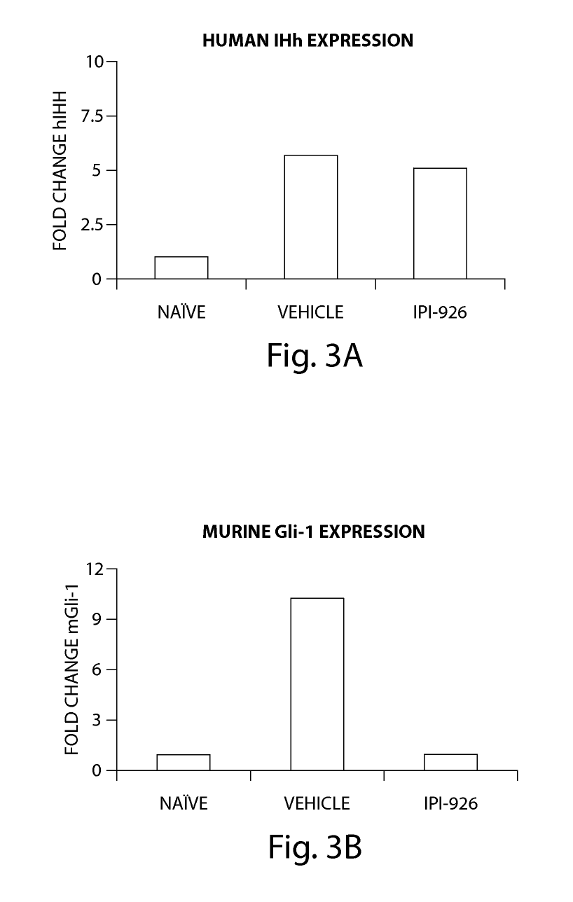

In the days following chemotherapy, there is an increase in the amount of stroma in the tumors following the cessation of chemotherapy, and that this stromal reaction resolves by 10-14 days (data not shown). Expression of Indian Hedgehog (IHH), one of the ligands in the Hh pathway, is induced as a consequence of therapeutic agent treatment (FIG. 3A). FIG. 3A is a bar graph depicting the change in human IHH expression in naïve, vehicle-treated and IPI-926-treated tumors. Expression of the stromal marker, Gli-1 was elevated in vehicle-treated control sample, and was inhibited after treatment with IPI-926 (FIG. 3B). These results confirm that there is an increase of Hh signaling, as measured by mouse Gli1, in the stroma of the tumors after chemotherapy, and that this signaling is inhibited by IPI-926 (FIGS. 3A-3B).

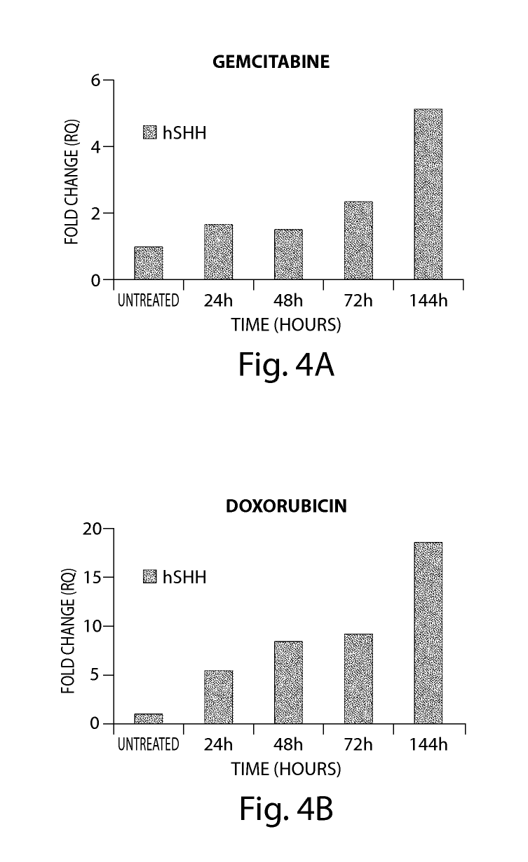

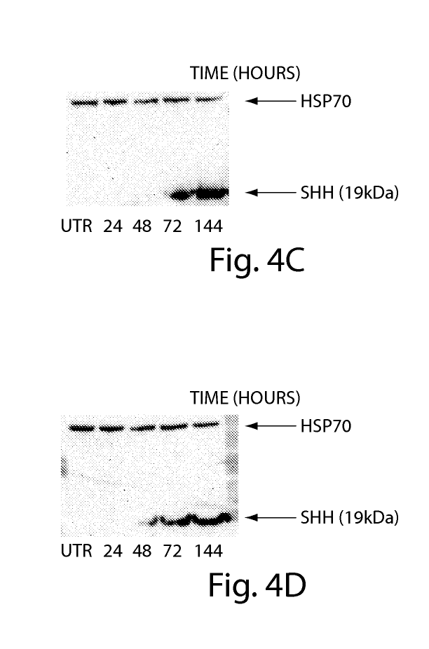

Similar results showing increases in Hh ligand expression in response to chemotherapy have been found in other tumor cells. For example, chemotherapy increases in Sonic Hedgehog (SHH) ligand expression in bladder cancer cells are depicted in FIGS. 4A-4D. More specifically, chemotherapy with Gemcitabine and Doxorubicin show an increase in expression over time as depicted in FIGS. 4A-4B, respectively. Photographs of representative corresponding Western blots are shown in FIGS. 4C-4D, respectively.

The experiments shown herein demonstrate that IPI-926 shows a marked growth inhibitory activity toward primary small cell lung (LX22) tumor recurrence following chemotherapy. Co-incident with IPI-926 activity at least the following activities are detected: Upregulation of IHH ligand expressed by the tumor cells; down regulation of murine Gli-1 in the tumor stroma; and a marked but transient stromal response. These experiments demonstrate the importance of continuity between therapeutic agent treatment and subsequent IPI-926 treatment. Thus, concurrent therapy (e.g., having at least some period of overlap between the therapeutic agent treatment and the IPI-926 treatment) is preferable over a sequential therapy with an interval between the therapeutic agent treatment and the subsequent IPI-926 treatment.

### Example 3

### Use of Hedgehog Inhibitor(s) as Maintenance Therapy

This example shows that IPI-926 can be used following cyto-reductive chemotherapy as maintenance therapy in several different therapeutic agent treatments.

To examine whether the effects of IPI-926 as effective maintenance therapy for a wide number of chemoresponsive tumor types, the effects of IPI-926 administered following different therapeutic agent treatments were examined in ovarian cancer, prostate cancer and non-small cell lung cancer.

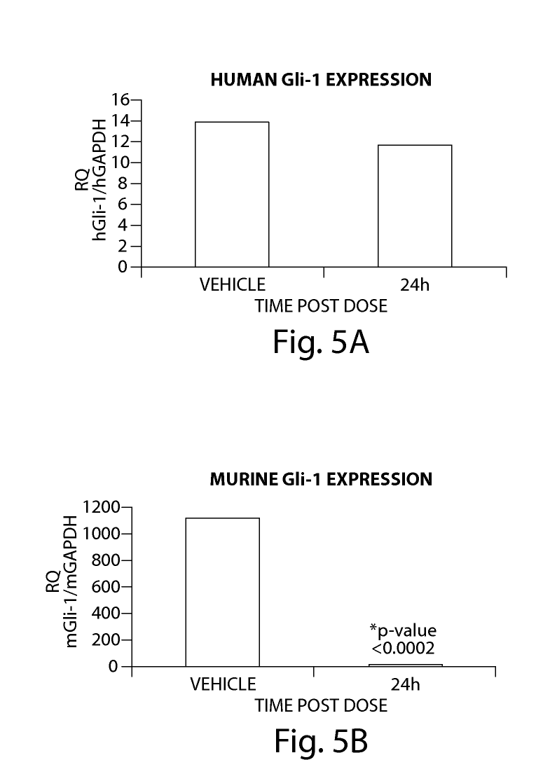

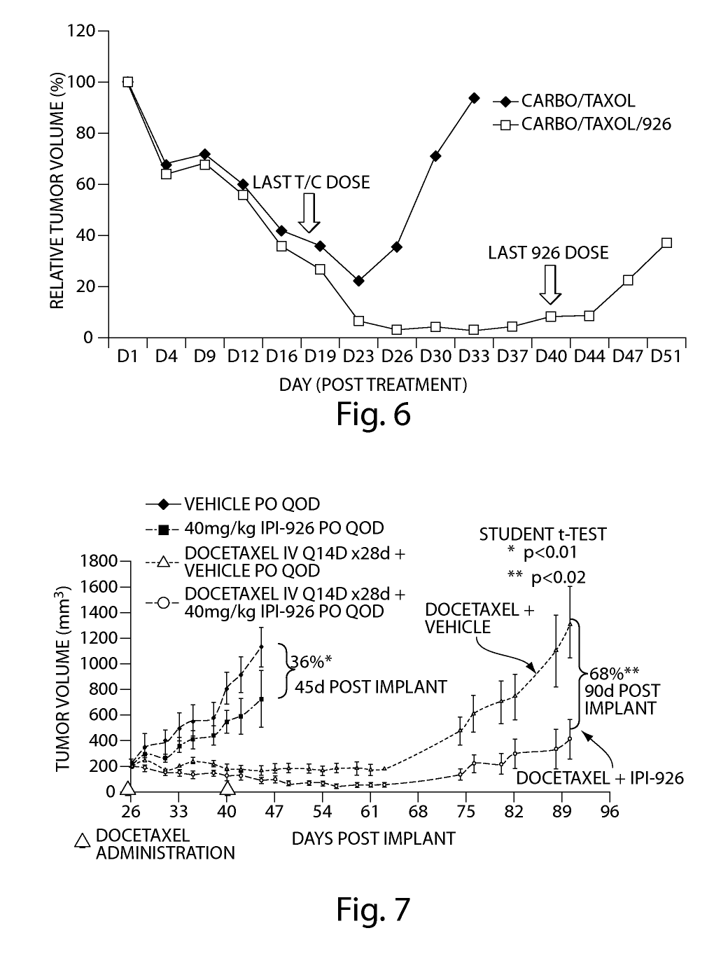

The effects of IPI-926 were examined following carboplatin/taxol combination chemotherapy in a series of primary ovarian cancer xenograft models. IPI-926 was shown to modulate mGLI-1 in primary xenograft model of ovarian cancer (FIGS. 5A-5B). FIG. 6 shows a maintained decrease in ovarian tumor volume by administration of IPI-926 following carboplatin/taxol chemotherapy.

In the OvCa studies:

1) IPI-926 was given daily, oral at 40 mg/kg

2) The taxol/carboplatinum was given:
a. Intraperitoneal Carboplatinum 50 mg/kg every 7 days
b. Intraperitoneal Paclitaxel 15 mg/kg every 7 days

Days of carboplatin/taxol and IPI-926 administration are indicated by the arrows. Tumor reoccurrence was detected after day 23 (about 4-5 days after cessation of carboplatin/taxol chemotherapy) in vehicle treated samples, whereas a prolonged duration of the tumor inhibition was observed in samples treated with IPI-926 following cessation of carboplatin/taxol chemotherapy. The inhibitory effects of IPI-926 persisted after discontinuing administration of IPI-926. Thus, IPI-926 can be useful as maintenance therapy in ovarian cancer.

The effects of IPI-926 were examined following docetaxel chemotherapy in a model of castration resistant prostate cancer. Prostate cancer is known to be a highly desmoplastic cancer, and one that preferentially metastasizes to bone. Sonic hedgehog ligand is expressed in clinical specimens and primary xenograft models. For example, human prostate cancer TMA revealed about 77% positive staining for sonic hedgehog ligand. These facts suggest that hedgehog might be involved in the pathogenesis of prostate cancer. FIG. 7 summarizes the effects of IPI-926 in LuCaP35V (Castration Resistant) primary prostate cancer model. Days of docetaxel and IPI-926 administration are indicated by the arrowheads at the indicated days post-implant. The following samples were tested: Vehicle control (administered orally once a day), 40 mg/kg of IPI-926 (administered orally once a day), docetaxel (administered intravenously Q14D for 28 days), or docetaxel (administered intravenously Q14D for 28 days) followed by 40 mg/kg of IPI-926, as shown in FIG. 7. Both vehicle control and IPI-926 alone showed a marked increase in tumor volume at the indicated time intervals post implant examined. Tumor reoccurrence was detected after cessation of docetaxel chemotherapy (see docetaxel+vehicle samples). A prolonged duration of the tumor inhibition was observed in samples treated with IPI-926 following cessation of docetaxel chemotherapy. Thus, IPI-926 can be useful as maintenance therapy in prostate cancer.

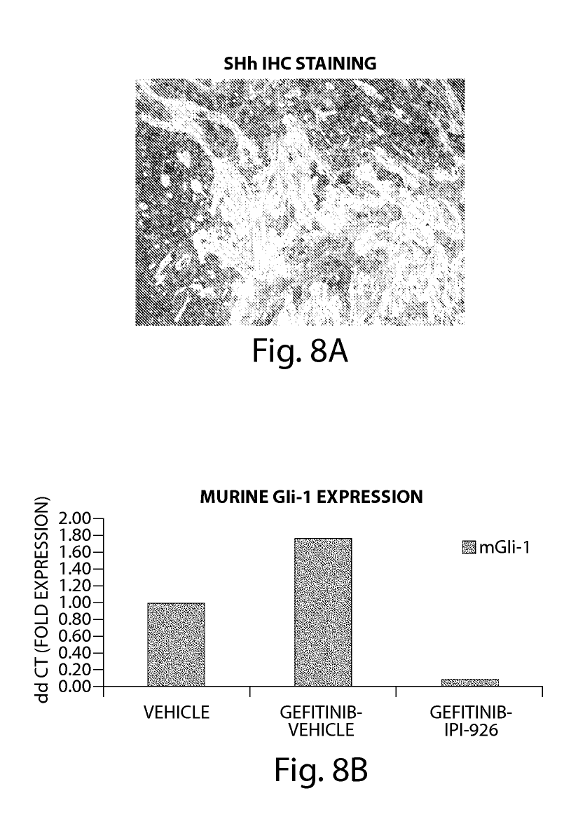

The efficacy of IPI-926 was evaluated when applied as a maintenance therapy following treatment of a xenograft non-small cell lung cancer model with a tyrosine kinase inhibitor. H1650 is a mutant EGFR xenograft model sensitive to Gefitinib in vivo. Sonic hedgehog ligand is detected by immunohistochemical staining (IHC) of sections of non-small cell lung cancer (FIG. 8A). IPI-926 was shown to inhibit mGLI-1 mRNA expression in lung tumor samples treated with IPI-926 in combination with Gefitinib, but not Gefitinib vehicle (FIG. 8B), thus demonstrating an effect of IPI-926 in the lung tumor and its microenvironment. FIG. 9 shows the activity of IPI-926 in H1650 xenograft following treatment with Gefitinib. The following samples were tested: Vehicle control; 40 mg/kg of Gefitinib administered orally for one week; 40 mg/kg of Gefitinib administered orally for one week followed by vehicle control; and 40 mg/kg of Gefitinib administered orally for one week followed by IPI-926 (administered once a day for three weeks). Vehicle control showed a marked increase in tumor volume at the indicated time intervals post implant examined. Tumor reoccurrence was detected after cessation of Gefitinib chemotherapy (see Gefitinib+vehicle samples). A prolonged duration of the tumor inhibition was observed in samples treated with IPI-926 following cessation of Gefitinib chemotherapy. Thus, IPI-926 can be useful as maintenance therapy in lung cancer.

Thus, IPI-926 can be used following cyto-reductive chemotherapy as maintenance therapy in a wide number of chemoresponsive tumor types, including ovarian cancer, prostate cancer and non-small cell lung cancer.

### Example 4

### Prevention of Tumor Metastasis Using Hedgehog Inhibitors

This example summarizes two similar studies (Study #1 and Study #2) showing that pretreatment with IPI-926 every-other day for between 7 and 14 days, limits the outgrowth and formation of L3.6pl P-lucky metastasis in a model of liver metastasis. Concurrently, with a decrease in metastasis burden, an overall survival benefit is observed with this pretreatment as well. Study 2 illustrates that although 14 days of pre-treatment alone can limit out-growth of metastasis; continued dosing after implantation provides a greater protective effect against the formation of metastasis as well as an overall survival benefit, greater than that observed when treatment is stopped on the day of implant.

### Study #1: Prevention of Liver Metastasis

### Experimental Design

Cells: L3.6pl is a pancreatic cancer cell line that has been tagged with the bioluminescence marker, luciferase, and is known to form metastasis in the liver (pl—pancreas to liver). Cells were cultured in RPMI+10% FBS and harvested on the day of implant. A single cell suspension was prepared in PBS at a concentration of 10 million cells per 1 ml of PBS. These cells were kept at 4° C. until implantation.

Model Procedure: Animals were anesthetized and prepared for surgery. The animal's spleen was exposed and 100 μl (1 million cells) of the L3.6pl single cell suspension was injected directly in the spleen towards the splenic vein (intra splenic injection). Once the injection was complete, the splenic artery and splenic vein were ligated, the spleen was excised, and the animals' wounds were closed. Post-op care and analgesia was given for 4 consecutive days and animals were monitored for recovery.

Prior to the procedure listed above 40 animals were separated into 4 groups consisting of 10 animals each. The groups were designated as seen in Table 1 below.

TABLE 1

| | | |
|---|---|---|
| Group 1 | N = 10 | Vehicle Treatment (Day 0) |
| Group 2 | N = 10 | Pre Treatment (Day-14) |
| Group 3 | N = 10 | Day of Treatment (Day 0) |
| Group 4 | N = 10 | Post Treatment (Day 7) |

Animals in group 1 (Vehicle), received IPI-926 vehicle every-other-day beginning on the day of the intra splenic injection procedure, continuing until the study end.

Animals in group 2 (Pre-Treatment), received IPI-926 every-other-day for 14 days, prior to the intra splenic injection procedure, continuing until the study end.

Animals in group 3 (Day-of-Treatment), received IPI-926 every-other-day beginning on the day of the intra splenic injection procedure, continuing until the study end.

Animals in group 4 (Post-Treatment), received IPI-926 every-other-day beginning 7 days after the intra splenic injection procedure, continuing until the study end.

Results Procedure: Animals were injected with 10 ml/kg of luciferin concentrated at 15 μg/ml via I.P. injection. This luciferin binds to the L3.6pl P-Lucky cells injected on day 0. Utilizing Calipers Xenogen© machine, this cell-luciferin association emits bioluminescence that can be read and quantified (total flux).

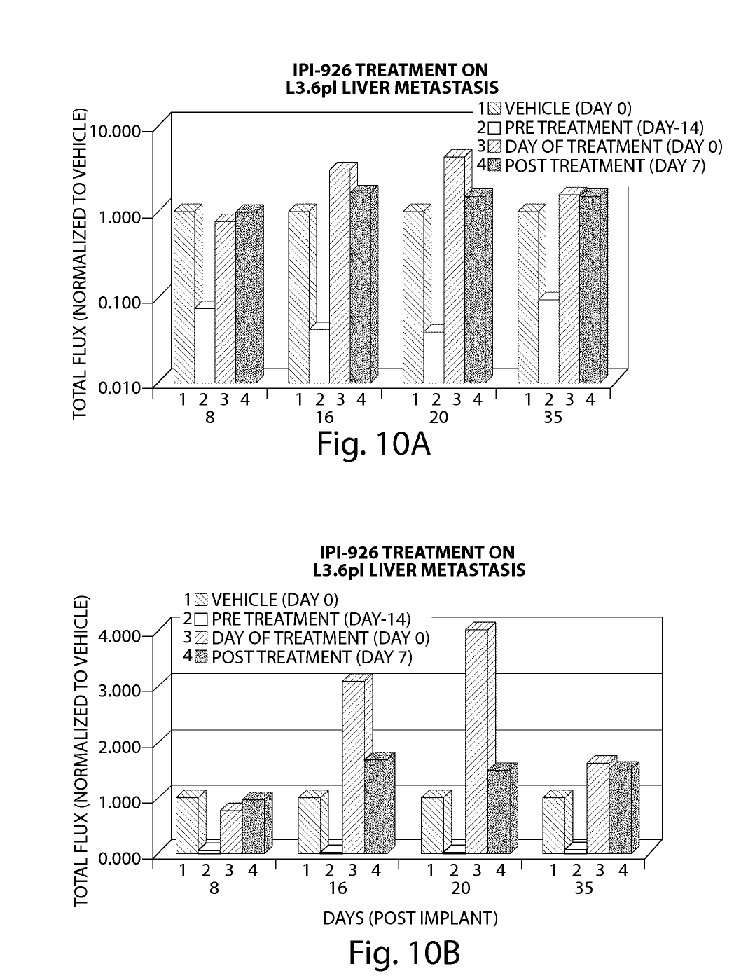

Results: The graphs in FIGS. 10A-10B represent the quantification, normalized on each day to the average of vehicle treated animals. This normalization was done using the formula: (1/(average flux of vehicle animals/average flux of group X (group being compared))). FIG. 10A shows the data on a log scale, and FIG. 10B shows this data on a normal scale.

The results summarized in FIGS. 10A and 10B shows that treatment with IPI-926 for 14 days prior to implant significantly reduces the growth and formation of metastasis within the liver. The reduction in flux, or bioluminescence, is 20-25 fold below vehicle. Day of implant treatment and post implant treatment has no detectable effect on take or growth of metastasis within the liver compared to vehicle treated animals.

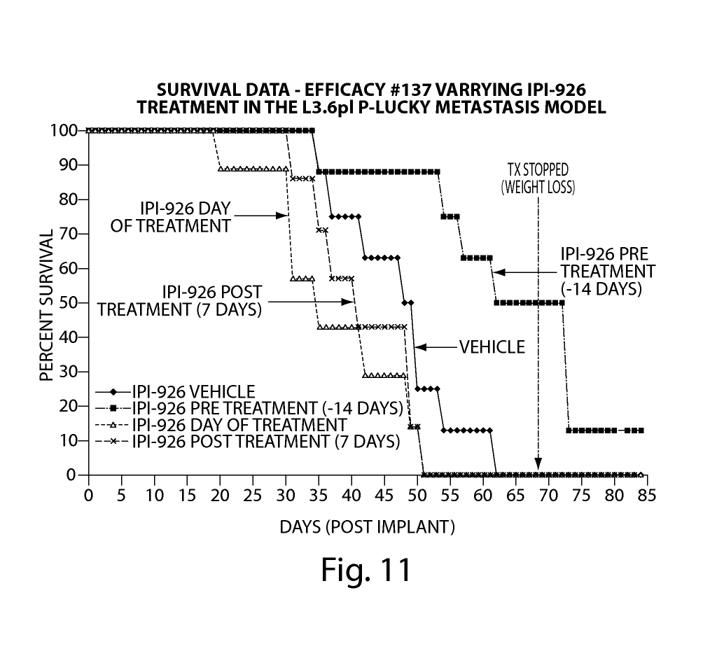

Survival Results: FIG. 11 represents the overall percent survival observed from each group within this study. Treatment with IPI-926 for 14 days prior to implant, doubles the overall survival rate compared to vehicle treated animals. This is likely a direct correlation and can be attributed to the reduction of the growth and formation of metastasis within the liver seen via Xenogen readings. Both the day of treatment group and the post treatment group had survival rates similar to vehicle treated animals.

Immunohistochemistry results of H&E staining performed on FFPE livers taken from 1 animal from each group prior to study end on day 21 show that vehicle treated, day of treated, and post treated groups all had visible metastasis and tumors cells present. In contrast, H&E staining from pre-treatment animal had no detectable tumor cells or metastasis.

### Study #2: Prevention of Liver Metastasis

Model Procedure: Animals were anesthetized and prepared for surgery. The animal's spleen was exposed and 100 μl (1 million cells) of the L3.6pl single cell suspension was injected directly in the spleen towards the splenic vein (intra splenic injection). Once the injection was complete, the splenic artery and splenic vein were ligated, the spleen was excised, and the animals' wounds were closed. Post-op care and analgesia was given for 4 consecutive days and animals were monitored for recovery.

Prior to the procedure listed above 48 animals were separated into 4 groups consisting of 8 animals each. The groups were designated as seen in the Table 2 below.

TABLE 2

| | | |
|---|---|---|
| Group 1 | N = 8 | Vehicle Treatment (Day 0) |
| Group 2 | N = 8 | Day of Treatment (Day 0) |
| Group 3 | N = 8 | Pre Treatment (Day −2) |
| Group 4 | N = 8 | Pre Treatment (Day −7) |
| Group 5 | N = 8 | Pre Treatmen (Day −14)-Treatment Stop |
| | | Day 0 |
| Group 6 | N = 8 | Pre Treatment (Day −14) |

Animals in group 1 (Vehicle), received IPI-926 vehicle every-other-day beginning on the day of the intra splenic injection procedure, continuing until the study end.

Animals in group 2 (Day-of-Treatment), received IPI-926 every-other-day beginning on the day of the intra splenic injection procedure, continuing until the study end.

Animals in group 3 (Pre-Treatment Day −2), received IPI-926 every-other-day starting two days prior to the day of the intra splenic injection procedure, continuing until the study end.

Animals in group 4 (Pre-Treatment Day −7), received IPI-926 every-other-day starting seven days prior to the day of the intra splenic injection procedure, continuing until the study end.

Animals in group 5 (Pre-Treatment Day −14—treatment stopped on Day 0), received IPI-926 every-other-day starting fourteen days prior to the day of the intra splenic injection procedure and ending on the day of implant.

Animals in group 6 (Pre-Treatment Day −14), received IPI-926 every-other-day starting fourteen days prior to the day of the intra splenic injection procedure, continuing until the study end.

Results Procedure: Animals were injected with 10 ml/kg of luciferin concentrated at 15 μg/ml via I.P. injection. This luciferin binds to the L3.6pl P-Lucky cells injected on day 0. Utilizing Calipers Xenogen© machine, this cell-luciferin association emits bioluminescence that can be read and quantified.

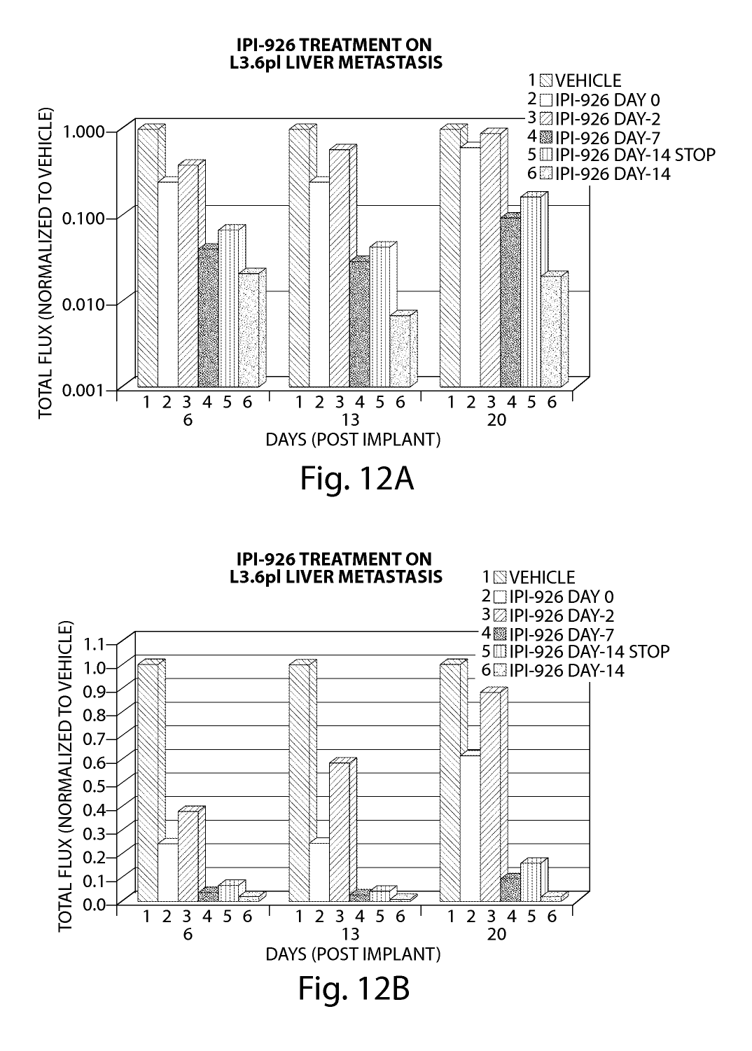

Results: FIGS. 12A-12B represent the results from the quantification, normalized on each day to the average of vehicle treated animals. This normalization was done using the formula: (1/(average flux of vehicle animals/average flux of group X (group being compared))). FIG. 12A shows the data on a log scale, and FIG. 12B shows the data on a normal scale.

Treatment with IPI-926 for 14 days prior to implant drastically reduces the growth and formation of metastasis within the liver. This reduction in flux, or bioluminescence, is 20-25 fold below vehicle. It is also noted that treatment with IPI-926 for 7 days prior to implant drastically reduces the growth and formation of metastasis within the liver. This reduction in bioluminescence is 10-15 fold below vehicle. Treatment with IPI-926 for 14 days prior to implant then stopping dosing similarly reduces the growth and formation of metastasis within the liver although growth is seen at the latest time point. This reduction in luminescence begins roughly 15 fold below vehicle in the early time point, and decreases to a 5 fold decrease in bioluminescence compared to vehicle, at the latest time point. Day of implant treatment and 2 days of pre treatment have no effect on take or growth of metastasis within the liver when compared to vehicle.

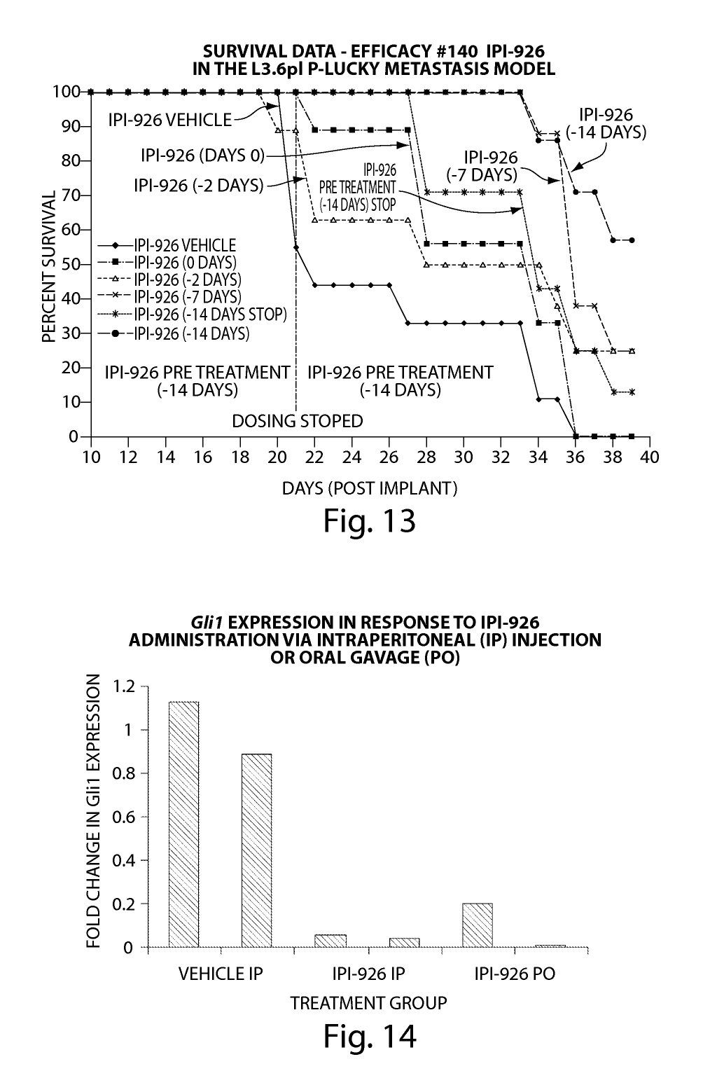

Survival Results: FIG. 13 represents the overall survival observed from each group within this study. Treatment with IPI-926 for 14 days prior to implant, increases the overall survival rate by at least a factor of 2. This is likely a direct correlation and can be attributed to the reduction of the growth and formation of metastasis within the liver seen via Xenogen. Similarly, 7 days of pre-treatment and 14 days of pre-treatment then stopping, also provides a survival benefit when compared to vehicle treated animals. Treatment starting on the day of implant, day 0, shows no benefit regarding overall survival. Pre-treatment beginning at day −2 provided limited survival benefits.

In summary, two similar studies (Study #1 and #2) have shown that pretreatment with IPI-926 every-other day for between 7 and 14 days, limits the outgrowth and formation of L3.6pl P-lucky metastasis in a model of liver metastasis. This is seen via bioluminescence readings captured via Xenogen. Concurrently, with a decrease in metastasis burden, an overall survival benefit is observed with this pretreatment as well. Study 2 illustrates that although 14 days of pre-treatment alone can limit out-growth of metastasis; continued dosing after implantation provides a greater protective effect against the formation of metastasis as well as an overall survival benefit, greater than that observed when treatment is stopped on the day of implant.

### Example 5

### IPI-926 is Active in Medulloblastoma Cells, Including Medulloblastoma Cells Resistant to Other Hedgehog Inhibitors

This Example shows that IPI-926 reduces tumor growth and the tumor-initiating capacity of medulloblastoma tumors, including cells with a point mutation that rendered them resistant to another Shh antagonist GDC-0449.

### 5.1 Summary

The Sonic hedgehog (Shh) pathway drives cancer progression in about 20-25% of medulloblastomas, a common type of pediatric brain cancer. Small molecule Shh pathway inhibitors have induced tumor regression in mice and patients with medulloblastoma; however, drug resistance rapidly emerges, in some cases via de novo mutation of the drug target. In this example, response and resistance mechanisms to IPI-926, in an aggressive mouse medulloblastoma model were evaluated. IPI-926 induced tumor reduction and significantly prolonged survival. The drug resistance encountered was not mutation-dependent and IPI-926 was found to be active in cells with a point mutation that rendered them resistant to another Shh antagonist GDC-0449.

Given the significant toxicities associated with standard medulloblastoma therapies, there is a strong need to improve treatment options. Novel therapies that target specific pathways underlying medulloblastoma genesis and progression are currently being developed. The progression of medulloblastoma treated with IPI-926, a small molecule that targets the hedgehog pathway by inhibiting Smoothened, was evaluated in an Shh-driven mouse medulloblastoma model using magnetic resonance imaging (MRI) to measure tumor effect, as well as survival endpoints. IPI-926 crossed the blood brain barrier, and displayed therapeutic efficacy at well tolerated doses. The significant activity of IPI-926 in cells resistant to GDC-0449 as well as the absence of genetic based resistance to IPI-926 indicates the utility of IPI-926 as a first or second line therapy for medulloblastoma.

### 5.2 Introduction

Medulloblastoma is a common malignant brain cancer in children. Recent genome-wide analyses revealed that medulloblastomas fall into four molecular categories; those driven by sonic hedgehog (Shh), those driven by Wnt, and two other subtypes for which the molecular drivers have not yet been identified (Kool et al., (2008) PloS ONE 3, e3088); Thompson et al., (2006) Journal of Clinical Oncology 24:1924-1931; Cho et al., 2010; Northcott et al., (2010) Neurosurg Focus 28(1):E6). Shh-driven tumors represent about 20-25% of medulloblastomas overall and are the predominant tumor type in infant and young adult medulloblastoma patients (Taylor et al. (2002) Nature Genetics 31: 306-410; Zurawel et al. (2000) Genes, Chromosomes and Cancer 28: 77-81; Pomeroy et al. (2002) Nature 415: 436-442; Northcott et al., (2010) Neurosurg Focus 28(1):E6). While long term survival for standard- and high-risk medulloblastoma patients is now greater than 70% and 50% respectively, this comes at a significant cost of toxicity due to surgery, radiation, and chemotherapy (Rossi et al. (2008) Clinical Cancer Research 14: 971-976; Packer et al.). The overall survival rates in the recurrent disease setting range from 9% to 26% with the median survival of two years (Zeltzer et al. (1999) Journal of Clinical Oncology 17: 832-845; Saunders et al., 2003; Bower et al., 2007). Shh pathway activation also drives several other types of cancer through cell autonomous oncogenic mechanisms or induction of micro-environment properties that provide a growth advantage to tumor cells (Katoh et al., (2009) Current Molecular Medicine 9: 873-886; Yauch et al. (2008) Nature 455: 406-410). Pathway inhibitors are being actively investigated for Shh-driven medulloblastoma in both the pre-clinical and clinical level.

To date, therapeutic candidates consist primarily of molecules that target the Smoothened protein. In normal Shh signaling, smoothened (Smo) is released from inhibition by the Patched (Ptch) receptor by surface binding of Shh. Smo then activates downstream Shh targets such as the Gli transcription factors. KAAD-cyclopamine, a modified plant alkaloid that targets Smo, induces remission in a mouse medulloblastoma model and causes apoptosis in primary human medulloblastoma cell cultures established from re-sected pediatric tumors (Berman et al., (2002) Science 297: 1159-1561). HhAntag, the first synthetic small molecule Smo antagonist reported, induces dramatic resolution of autochthonous brain tumors and flank medulloblastoma xenografts in a Ptch1^{+/-}; p53^{-/-} mouse model (Romer et al., (2004) Cancer Cell 6: 229-240). Newer generation synthetic small molecules are now being used in patients. GDC-0449 was reported to induce significant reduction in tumor burden in an adult medulloblastoma patient with Shh-driven disease, and clinical responses in pediatric patients with Shh-driven medulloblastoma have been reported (Yauch et al. (2009) Science 326: 572-574). The same molecule induces tumor regression in basal cell carcinoma patients (Von Hoff et al. (2009) N. Engl. J. Med. 361: 1164-1172). While these are important first steps toward effectively targeting the Shh pathway in cancer, responses are sometimes short-lived due to the emergence of drug resistance. It remains to be determined whether these drugs confer a survival benefit to medulloblastoma patients.

As is the case with many targeted therapies that interact with a single protein in the cell, point mutations that confer drug resistance and provide a growth advantage to cancer cells have been reported in response to Shh antagonism (Yauch et al. (2009) Science 326: 572-574). This mechanism of resistance has been observed both in mice and humans. In addition, like many oncology drugs, GDC-0449 is a p-glycoprotein (Pgp) substrate, which can theoretically lead to drug resistance through selective growth advantage of cells that inhibit drug entry, or elevated expression of ATP-Binding Cassette (ABC) multidrug efflux transporters at the blood brain barrier. Unfortunately, these are nearly universal challenges associated with treating brain cancer and previous attempts to block ABC transporters as a chemosensitizing measure have not yet achieved clinical success.

IPI-926 is a selective, potent, small molecule that targets the Hh pathway by inhibiting Smo. IPI-926 is orally bioavailable, has a long plasma half-life, a long duration of action, and has demonstrated biological activity in multiple preclinical animal models of cancer (Tremblay et al., (2009) Journal of Medicinal Chemistry 52: 4400-4418; Olive et al., (2009) Science 324: 1457-1461). In this study, IPI-926 activity in a very aggressive mouse medulloblastoma model was assessed. This mouse medulloblastoma model has a targeted loss of the Shh pathway negative regulator, Patched 1 (Ptch1), in Math1-expressing cerebellar granule neuron precursors (Math1-cre/Ptc^{C/C}) (Yang et al. (2008) Cancer Cell 14: 135-145). At doses that were well tolerated, rapid autochthonous brain tumor regression accompanied by restoration of normal neurologic function was observed in mice that were generally impaired at the time of study enrollment. Survival from the time of entry was increased 5-fold by the most effective dosing regimen. At the time of tumor progression, there was no evidence of genetic mutations that rendered the cancer cells resistant to IPI-926. However, there was a modest increase in Pgp, indicating one possible mechanism by which the cancer cells might evade IPI-926-mediated Shh pathway suppression.

### 5.3 Experimental Procedures

### Generation and Maintenance of Conditional Patched1 Null (Ptc^{C/C}) Mice

Transgenic mice were maintained in accordance with the NIH Guide for the Care and Use of Experimental Animals with approval from our Institutional Animal Care and Use Committee. Conditional Patched1 null mice (Ptc^{C/C}) were generated on a mixed background by breeding mice homozygous for the floxed Ptch1 allele (Adolphe et al., (2006) Cancer Res 66: 2081-2088) to Math1-Cre mice, as previously described (Yang et al. (2008) Cancer Cell 14: 135-145). Mice were genotyped by PCR using genomic DNA using the following primers:

| Ptch1-Floxed (Fwd): | |
|---|---|
| CCACCAGTGATTTCTGCTCA; | (SEQ ID NO: 1) |
| Ptch1-Floxed (Rvs): | |
| AGTACGAGCCATGCAAGACC; | (SEQ ID NO: 2) |
| Cre (Fwd): | |
| TCCGGGCTGCCACGACCAA; | (SEQ ID NO: 3) |
| Cre (Rvs): | |
| GGCGCGGCAACACCATTTT. | (SEQ ID NO: 4) |

### IPI-926 Dose Administration

Animal experiments were performed in accordance with the NIH Guide for the Care and Use of Experimental Animals with approval from our Institutional Animal Care and Use Committee. 100% penetrance of medulloblastoma in the Ptc^{C/C} mice was observed, with all mice displaying symptoms of tumor formation by the time of weaning. Ptc^{C/C} mice (age ranging from 21-36 days) were randomized to receive either IPI-926 (Infinity Pharmaceuticals) or vehicle control (5% (2-Hydroxylpropyl)-B-Cyclodextrin (HPBCD), Sigma Aldrich) administered via intraperitoneal (IP) injection. IPI-926 was originally optimized for oral bioavailability, and IP administration was equally effective at achieving Shh pathway inhibition in Ptc^{C/C} medulloblastomas (FIG. 14). FIG. 14 shows inhibition of Gli1 expression in response to IPI-926 administration via intraperitoneal (IP) injection or oral gavage (PO).

### Ptc^{C/C} Medulloblastoma Allografts

Animal experiments were performed in accordance with the NIH Guide for the Care and Use of Experimental Animals with approval from the Institutional Animal Care and Use Committee. Freshly excised medulloblastoma tumors from symptomatic Ptc^{C/C} mice were placed in cooled phosphate buffered saline (PBS), minced with a scalpel, and filtered through a 100 μm cell strainer (BD Bioscience, San Jose, Calif.). The cells were pelleted at 1000 rpm for 5 minutes at 4° C., and resuspended in equal parts DMEM and Matrigel (BD Biosciences, San Diego, Calif.). Recipient mice (wild type littermates) were anesthetized with isoflurane and a suspension of 1×10⁶ cells in total volume of 200 μL was injected subcutaneously into the flank using a 30G needle. Tumor growth was measured in two dimensions using digital calipers every 24-48 hours, and the tumor volumes were calculated according to the following formula: 0.5×length×width², with width being the smaller of the two dimensions measured. Tumors greater than 2.5 cm in length were harvested and either snap-frozen or fixed in 10% formalin.

### Tumor Pathology

Mice were euthanized using CO₂ inhalation, brains were removed and tissue snap frozen for RNA studies or fixed in 10% buffered formalin and processed for histopathological examination. Formalin-fixed tissues were paraffin embedded, cut into 4 um sections and stained with Haematoxylin and Eosin using standard methods.

Tumor and brain tissue from the cohort of mice analyzed by MRI were processed using a novel method that preserved the tissue while maintaining the spatial integrity of the brain and ventricular spaces within the skull. This technique enabled good histologic comparison to MRI images and analysis of secondary pathologic changes such as hydrocephalus. Whole brains within the skull were fixed in 10% buffered formalin, decalcified using Formical 4 (Decal Chemical Corporation) and processed for paraffin embedding. Tissues were sectioned along the horizontal plane to match MRI orientation. A cohort of samples was serially sectioned through the entire brain (up to 150 sections per animal) and H&E-stained to generate computerized three-dimensional (3D) renderings of the tumors. Tissue sections were digitally scanned at 5× magnification using the TissueFax scanning platform (TissueGnostics, Vienna, Austria) and images captured with a Pixelink digital camera. Images were stitched using the TissueFax software and stacked and aligned using the StackReg function of the imaging program ImageJ. Imaris was used to process each of the stacks into a 3D model. These models validated the MRI-based renderings (details below) and provide an additional tool for assessing tumor volume at a single end point.

Magnetic Resonance Imaging and Cholorotoxin: Cy5.5 (Ctx:Cy5.5) imaging analysis

Magnetic resonance imaging (MRI) was performed using a 3 Tesla MRI system (Philips Achieva, Philips Healthcare, Andover, Mass.) and a custom mouse head coil. Serial MR scans were performed using a 35 minute coronal high-resolution T2-weighted sequence (TE=110 ms, TR=2000 ms, bandwidth=212, 2 NEX or signal averages, a matrix of 256×256 pixels in-plane, slice thickness of 320 microns and an interslice gap of 160 microns). Mice were scanned under halothane anesthesia at enrollment, after 3 weeks of treatment, and after 6 weeks of treatment. Ctx:Cy5.5 bioconjugate (Tumor Paint) was given by intravenous tail vein injection one day prior to animal sacrifice. Biophotonic images were obtained using the Xenogen Spectrum imaging system (Caliper Life Sciences) as previously described (Veiseh et al. (2007) Cancer 67: 6882-6888).

DICOM images were exported from the MRI scanner to a web-based repository (BioScribe) and then imported into ITK-SNAP (version 2.0, available on the world wide web at itksnap.org). Images were first windowed to accentuate tumor/brain contrast, easily observed on the T2-weighted scans. Images demonstrated diffuse cerebellar involvement with striking posterior fossa enlargement and loss of foliar pattern. Effacement of the fourth ventricle was accompanied by lateral and third ventriculomegaly and transependymal CSF flow. Tumor and enlarged cerebellum was seen to herniate into the internal auditory canals (IAC) bilaterally. After each imaging time-point, using the manual tracing tool, tumor tissue (including the IAC components) was painted in three planes excluding frank cerebrospinal fluid or cystic regions. The resultant segmentation file was saved for later use. Three-dimensional surface-rendered reconstructions were then performed and saved in two standard projections for each tumor analyzed, with the aim of delineating a consistent view of the tumor for comparison between pre- and post-treatment scans and between treatment groups. Automated pixel counting multiplied by image pixel dimensions yielded volumetric measures for each segmentation analysis dataset. In addition to quantitative analysis, pre and post-treatment scans were compared for evaluation of secondary changes such as hydrocephalus, prominent extra-axial spaces, cystic change/necrosis in treated tumor and any evidence of hemorrhage.

### Gene Expression Analysis

Pharmacodynamic activity of IPI-926 in Ptc^{C/C} tumors was confirmed by analysis of Gli1 mRNA by RT-PCR. Mice were treated daily with 20 mg/kg/dose IPI-926 for 2 days, 2 weeks or 6 weeks and tumor tissue isolated and snap frozen 24 hours after the last dose. Total RNA was extracted using the Qiagen RNeasy Plus Kit and converted to cDNA using the Taqman Reverse Transcription kit (ABI). Quantitative Real Time PCR was set up using Taqman Master Mix and run on the Applied Biosystems 7300HT Real-Time PCR (384-well qPCR) System. Taqman primers for mouse Gli1 and Gapdh controls were used (ABI). Data was analyzed using SDS2.3 software (ABI). All conditions were run in triplicate and normalized to mouse Gapdh controls. Expression of IPI-926 treated (n=3 per time point) samples were normalized to vehicle control (n=3 per time point) samples.

### Immunohistochemistry and Immunofluorescence

4 μm paraffin-embedded cerebellar sections from Ptc^{C/C} tumors were stained with monoclonal antibodies recognizing Gli1 (1:250, Novus Biologicals, Littleton, Colo., USA), Ki67 (1:200, Novo Castra, Burlington, Ontario, Canada), BrdU (Accurate Chemical and Scientific Corporation, Westbury, N.Y.), activated caspase 3 (1:200, Cell Signaling Technology, Inc., Beverly, Mass.), Pgp (1:100, C219, Covance Research Products, Dedham, Mass.) and ABCG2/BCRP (1:50, Abcam). Secondary antibodies were applied according to the Vectastain Elite avidin-biotin complex method instructions and detection was carried out with 3,3′-diaminobenzidine reagent (Vector Laboratories, Burlingame, Calif.). Sections were visualized with a Zeiss Axioscope 40 microscope and images were captured with a Qimaging MicroImager II digital camera.

For double immunofluorescent staining, cerebellar sections from Ptc^{C/C} tumors were stained with antibodies recognizing Pgp and Gli1 using the same antibody concentrations for each. The M.O.M immunodetection kit (Vector Laboratories) was used to block nonspecific binding of mouse primary antibody. Incubation with anti-Pgp antibody was performed overnight at 4° C., followed by secondary antibody for 2 h at room temperature. The nuclei were counterstained with DAPI mounting media (Vector Laboratories), and the slides were observed using a Zeiss Axioscope 40 microscope and images were captured with a Qimaging MicroImager II digital camera.

### HPLC/Mass Spectrometry

IPI-926 drug levels in tumor and brain samples were determined as described previously (Olive et al., (2009) Science 324: 1457-1461). Briefly, samples were homogenized in 4 volumes of CAN:PBS buffer and homogenized using a Geno/Grinder from SPEX CertiPrep (Metuchen, N.J.) for 2 minutes. Homogenates were then filtered using 0.45 mM low binding hydrophilic multiscreen solvinert late (Millipore) and collected in a 96-well plate. The tissue filtrates were diluted 1:1 and IPI-926 levels were determined. Sample analysis was performed on an Agilent 1200 from Agilent Technologies (Santa Clara, Calif.) coupled with an API-4000 mass spectrometer from Applied Biosystems (Foster City, Calif.) for detection. Data were acquired and processed using the software Analyst 1.4.1 (Applied Biosystems). Sample concentrations, as measured by their peak area ratios (analyte divided by internal standard), were determined from the calibration curves.

### DNA Sequencing Analysis

Frozen tumor samples were lysed in a Geno/Grinder 2000 (SPEX CertiPrep) followed by DNA isolation using a QIAamp DNA mini kit (Qiagen). Samples were quantitated with a Nanodrop 2000c (Thermo). PCR primers were designed with Primer3 and incorporated either M13 forward (TGTAAAACGACGGCCAGT (SEQ ID NO: 5)) or reverse (CAGGAAACAGCTATGAC (SEQ ID NO: 6)) priming sites. Forward primers for SMO exons 1-12 (each prefixed with M13F):

| | |
|---|---|
| AAGCTGGCCCCAGACTTTC, | (SEQ ID NO: 7) |
| GCATAAGGCAACCCTTAGCA, | (SEQ ID NO: 8) |
| GCCCTATGAGGTAGGGGCTA, | (SEQ ID NO: 9) |
| CACCAGGACATGCACAGCTA, | (SEQ ID NO: 10) |
| AGCATTGCCCTGTTGTGTTC, | (SEQ ID NO: 11) |
| CTATGCCTTGATGGCTGGAG, | (SEQ ID NO: 12) |
| AGGCTCTGTCCCAGTTACCG, | (SEQ ID NO: 13) |
| TGTAGCCACCCTGGACTCAG, | (SEQ ID NO: 14) |
| CCATGAGAATCACGCAGTGG, | (SEQ ID NO: 15) |
| CTGTGAAGGCCTCAGCTCCT, | (SEQ ID NO: 16) |
| GCTCCAGGGTGGAATCTCTC, | (SEQ ID NO: 17) |
| ACCTGAAGGAGATGCCAAGG. | (SEQ ID NO: 18) |

Reverse primers for SMO exons 1-12 (each prefixed with M13R):

| | |
|---|---|
| CAACAGTTTGAGGCCTGAGC, | (SEQ ID NO: 19) |
| GCTTGACAACCATGCTCCAT, | (SEQ ID NO: 20) |
| AGCCACAAAGGTGGCCTAAA, | (SEQ ID NO: 21) |
| GGACACAGGTCGGATTTGAA, | (SEQ ID NO: 22) |
| CCAGCACGGTACCGATAGTTC, | (SEQ ID NO: 23) |
| GAACCTTGGTCATGGCTTTG, | (SEQ ID NO: 24) |
| CCCCTTCTCAGAGGGAGTTG, | (SEQ ID NO: 25) |
| ACCTGCTCCTGTGCATTGAC, | (SEQ ID NO: 26) |
| GGCTCCTGTGGCTCCTACTT, | (SEQ ID NO: 27) |
| CAGAGAAGAAGGAAGAGAGAGCAA, | (SEQ ID NO: 28) |
| CACTGTCAGGGGGACAAAGA, | (SEQ ID NO: 29) |
| CAGACACTTGGCCCACAGAC. | (SEQ ID NO: 30) |

100 ng gDNA was used in a 50 ul PCR reaction with 0.2 uM of each primer and Platinum PCR Supermix High Fidelity (Invitrogen). PCRs were run on a Dyad DNA Engine (MJ Research/Bio-Rad) using the following conditions: 95 degrees Celsius (° C.) for five minutes followed by 35 cycles of 95 degrees for 30 seconds, 60 degrees for 30 seconds and 68 degrees for 45 seconds, the program ended with a final extension step of 68 degrees for ten minutes. A portion of the reaction was visualized on e-gels (Invitrogen) and the remainder was sequenced by the Sanger method (GeneWiz, Cambridge Mass.). Mutations were identified using Mutation Surveyor version 3.23 (SoftGenetics, State College Pa.). Mutations were called only if found in reads in each orientation.

### Gli-Luciferase Reporter Assay

Wild type human SMO was subcloned into pcDNA3.1 from an expression construct in pCMV6 (Origene #SC122724). D473H SMO was then generated by site-directed mutagenesis using the Stratagene QuikChange kit (Agilent #200519) and sequence verified.

Gli-Luciferase reporter assays were performed as described. (Yauch et al. (2009) Science 326: 572-574) Briefly, C3H10 T1/2 cells (ATCC, #CCL-226) were plated in six-well plates at 1×10 to 5th cells per well in BME (Gibco #21010) with 10% FBS (Hyclone #SH30070.03), 2 mM Glutamine (Gibco #25030) and 50 units penicillin/50 ug streptomycin (Gibco #15140). The next morning, cells were transfected with 400 ng SMO expression construct, 400 ng 8× Gli-Luc, and 200 ng pRL-TK per well with GeneJuice transfection reagent (Novagen #70967). Cells in each well were lifted six hours later and replated into four wells of a 12 well plate and allowed to attach overnight. Medium was then changed to low (0.5%) serum and compounds were added in quadruplicate in a range of concentrations. After a 48 hour incubation, firefly and renilla luciferase were assayed using the Promega Dual-Glo Luciferase Assay System (Promega #E2940) and ratios were used to determine percent of control using Prism graphing software.

### Statistical Considerations

Studies were designed to detect differences in event rates that approximately corresponded to a doubling of median improvement in survival of (3 weeks) with 90% power based on simulated power experiments. These calculations assume a vehicle median survival rate of 3 weeks, 12 animals per arm and that a level 0.05 (two-side logrank test statistic; Kalbfleisch et al., 2002) would be used to test differences between arms. Survival analyses used animal death times and censoring times when animals were sacrificed at approximately 6 weeks or as otherwise stated. The Kaplan-Meier (Kaplan et al., 1958) method was used to estimate survival distributions, and differences between groups were assessed using the logrank test statistic. All P-values quoted are two sided. Ultimately, there were 4 comparisons of groups based on survival; therefore, the Bonferroni multiple comparison adjusted P-value is 0.0125. All statistical analyses were performed using the R suite of software facilities (available on the world wide web at r-project.org).

### 5.3 Results

### Mouse Model Selection and Pharmacodynamic Studies

Two mouse medulloblastoma models were considered for these studies. In the Smo/Smo model, the constitutively active Smoothened (SmoA1) transgene is driven by a fragment of the mouse NeuroD2 promoter (Hallahan et al., (2004) Cancer Research 64: 7794-7800). Over 90% of Smo/Smo mice develop subclinical, localized medulloblastomas by one month of age and they typically become symptomatic and moribund 3-5 months later (Hatton et al., (2008) Cancer Research 68: 1768-1776). In this model, Smoothened is activated by a W539L point mutation (SmoA1) in the seventh transmembrane domain (Xie et al. (1998) Nature 391: 90-92; Taipale et al., (2000) Nature 406:1005-1009). While this model is ideal for many pre-clinical medulloblastoma therapeutic trials, it was previously reported that the introduced SmoA1 point mutation reduced the affinity of cyclopamine for Smoothened in tissue culture cells (Taipale et al., (2000) Nature 406:1005-1009). Tests were performed to determine whether doses of IPI-926 that were effective in other mouse model studies were sufficient to block the Shh pathway in Smo/Smo mouse tumors. The expression of Gli1, a downstream target of Shh, was unaffected by IPI-926, indicating that like cyclopamine, IPI-926 is not active against Smoothened bearing the A1 point mutation. In addition, there was also no detectable effect of IPI-926 on proliferation or apoptosis in this mouse model (not shown). Therefore, a different medulloblastoma model was used.

The conditional Patched1-null mice (hereafter referred to as Ptc^{C/C}) were generated by interbreeding with Math1-Cre animals, lacking both alleles of Patched1 (Ptch1) specifically in cerebellar granule neuron precursors (GNPs) (Yang et al. (2008) Cancer Cell 14: 135-145). The mice have no other engineered modifications of oncogenes or tumor suppressors. The Ptc^{C/C} model is notable for massive hyperproliferation of granule cells throughout the cerebellum and the evolution of highly aggressive tumors that are clinically evident as early as 3 weeks of age and induce death within weeks after becoming symptomatic. These are multifocal tumors that have malignant tumor initiating potential evidenced by growth of transplanted tumors in wild type recipient mice. This model poses challenges for pre-clinical drug studies because mice are moribund soon after weaning and cerebellar granule neuron precursors exhibit unbridled Shh-driven proliferation.

In initial studies in the Ptc^{C/C} mice intracranial pressures were sufficiently high in some mice that brainstem herniation into the spinal canal and subsequent death occurred by tipping the head back for gavage feeding. Pharmacodynamic studies were conducted with intraperitoneal (IP) drug injection as well as oral gavage to see whether the IP route offered a safe and effective alternative to gavage drug administration. Both oral and IP routes induced approximately 90% reduction in Gli1 mRNA levels (FIG. 14), so IP administration was used for all subsequent studies.

### IPI-926 Induces Clinical Remission and Extends Survival of Mouse Medulloblastoma

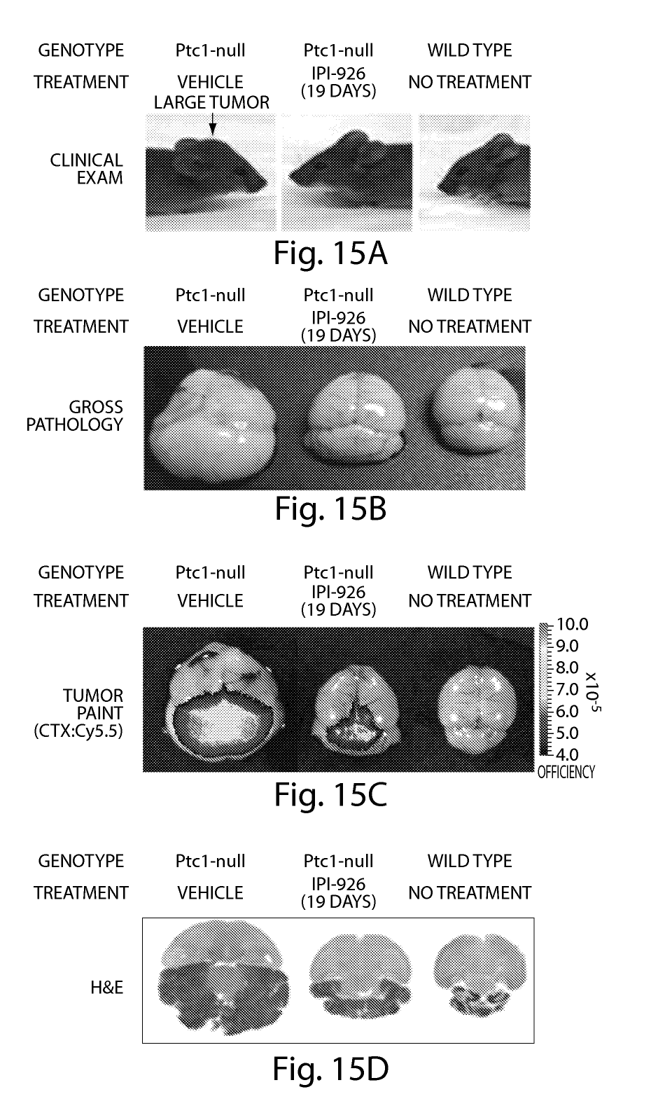

The Smo inhibitor IPI-926 causes dramatic regression of mouse medulloblastoma and resolution of advanced clinical symptoms. The efficacy of IPI-926 was evaluated in a pilot study using Ptc^{C/C} mice. A dramatic response to IPI-926 was apparent by gross pathology (FIG. 15B), ex vivo imaging with Tumor Paint (Ctx-Cy5.5), a tumor-tracking molecular imaging agent (FIG. 15C), and haematoxylin and eosin (H&E) stained tissue sections (FIG. 15D).

More specifically, a pilot study was performed to evaluate the efficacy of IPI-926 in 21-day old Ptc^{C/C} mice with clinical evidence of medulloblastoma (dome-shaped skull due to tumor burden). Three-week old mice symptomatic for medulloblastoma were randomized to receive daily intraperitoneal IPI-926 (20 mg/kg/dose, n=3) or vehicle (n=2) for 19 days. By seven days of treatment, treated mice began demonstrating substantial tumor regression, and a full resolution of clinical symptoms was evident by 19 days of treatment (FIG. 15A). Compared to a representative vehicle-treated mouse with a large tumor (left panels) and a wild-type littermate with no tumor (right panels), a representative mouse treated with IPI-926 (center panels) showed complete resolution of clinical symptoms after 19 days of IPI-926 treatment (FIG. 15A). The arrow in FIG. 15A denotes the bulging skull, symptomatic evidence of medulloblastoma formation. In contrast, vehicle treated mice showed progressive tumor growth.

Analysis of gross tumor pathology following treatment demonstrated a strong response to IPI-926 therapy, with decreased cerebellar tumor size in treated mice (FIG. 15B). Imaging with Tumor Paint (Ctx:Cy5.5), a tumor-tracking molecular bioconjugate (Veiseh et al. (2007) Cancer Res. 67: 6882-6888), exhibited a reduction in tumor burden in IPI-926 treated mice (FIG. 15C), and histopathological analysis of cerebellar tumor sections also revealed a decrease in tumor burden and regions of nuclear condensation and cell debris. (FIG. 15D). The foliation pattern in the cerebellum was completely obliterated in vehicle treated tumors, whereas IPI-926 treated animals manifested regions of tumor cell death, as indicated by pyknotic nuclei with retention of normal cerebellar architecture.

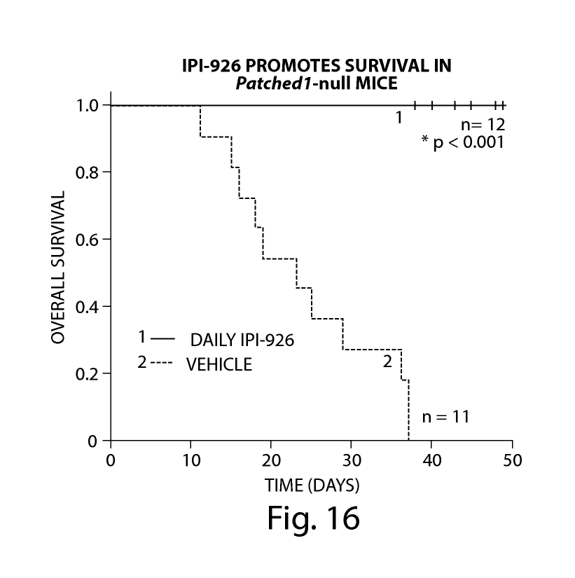

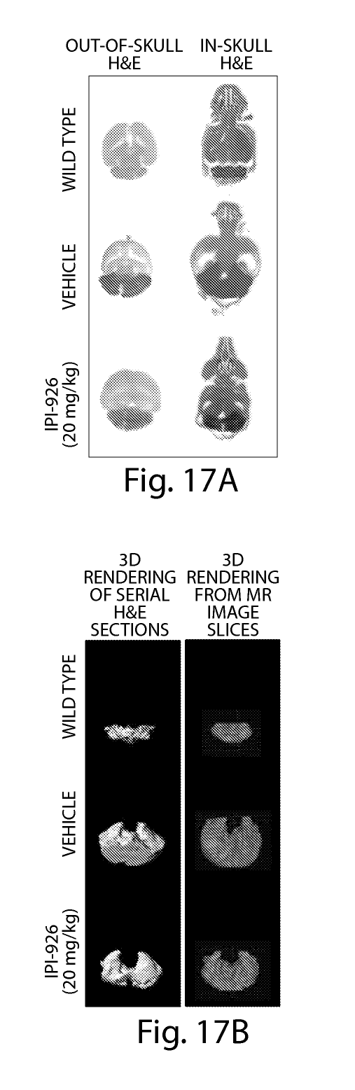

Given these promising results, a larger scale study was performed. The duration of therapy was extended. Study animals received 6 weeks of daily IPI-926 (n=12) versus vehicle control (n=11). Three- to five-week-old mice symptomatic for medulloblastoma were randomized to receive vehicle or intraperitoneal IPI-926 (20 mg/kg/dose). Tumor growth was monitored twice weekly, and mice were sacrificed for histopathological analysis at a 6 week end point or earlier as required by disease burden. Kaplan-Meier analysis demonstrates that all mice receiving daily IPI-926 (20 mg/kg, line) (shown as #1) survived, while all vehicle-treated mice (line shown as #2) succumbed to their disease prior to the six-week time point (P<0.001). Kaplan-Meier survival curves and P values were generated using the survival package from R (FIG. 16). These results show that IPI-926 dramatically improves survival in the Ptc^{C/C} medulloblastoma model.

Clinical symptoms were resolved in many of the IPI-926 treated mice, accompanied by restored neurologic function and increased activity. The profound difference between 100% survival and neurologic recovery in IPI-926-treated mice compared to 100% death in vehicle-treated mice prompted in depth analyses of tumor response.

### Magnetic Resonance Imaging (MRI) Detects Sub-Clinical Disease Progression

In human brain tumor clinical trials, non-invasive MRI is used to detect disease progression earlier than can be detected by clinical exam or survival endpoints. Brain tumor volume was assessed by MRI at 3 week intervals during this study. Motion artifact from breathing, sensitivity of brain tumor bearing mice to anesthesia and other technical challenges were overcome. One unanticipated challenge was that initially, MRI-estimated tumor volumes did not match those predicted by histological analyses of resected tumors at the end of the study. The discrepancy was due to dramatic changes in brain shape that occurred when the organ was removed from the restrictive confines of the skull for histological processing (FIG. 17A). A technique was developed for preserving the brain within the skull. Tissues processed within the skull were from tumors that were monitored via MRI during the course of the 6-week IPI-926 study. These tissues were sectioned along the horizontal plane to match the MRI orientation. A cohort of samples was serially sectioned through the entire brain and H&E-stained to generate computerized 3D renderings of the tumors. Images were stitched using the TissueFax software and stacked and aligned using the StackReg function of the imaging program ImageJ. Imaris was used to process each of the stacks into a 3D model. These H&E-based 3D tumor models are matched to representative H&E stained slides from each sample as well as to the MRI-generated volume model for the same sample in the panels in FIG. 17B. The H&E-based volume models validated the MRI-based renderings and provide an additional tool for assessing tumor volume at a single end point. With this method, three dimensional reconstruction of histologically stained brain sections matched MRI findings for tumor shape and volume and also for ventricular size and shape, which reflects the degree of hydrocephalus in tumor-bearing mice (FIG. 17B). The experiments suggest that MRI, rather than histology, should be the standard and that in-skull fixation should be used to accurately capture tumor and brain data for histological analyses. Thus, in-skull tissue processing preserves intracranial integrity enabling accurate 3D tumor volume rendering and analysis of pathology.

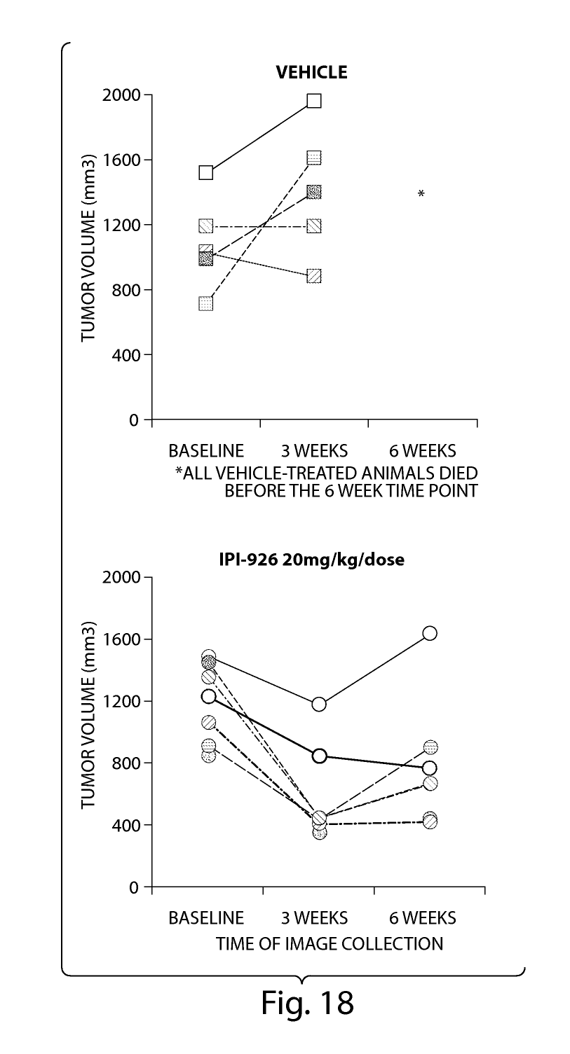

MRI scans demonstrate decreasing tumor volumes at multiple treatment time-points during daily IPI-926 administration, but indicate tumor progression despite prolonged therapy. MRI analyses showed that IPI-926 treatment induced substantial tumor regression after three weeks of daily administration (summarized in FIG. 18). Hydrocephalus was commonly noted in vehicle-treated mice, as well as enlarged ventricles and trans-ependymal cerebral spinal fluid (CSF) flow resulting from fourth ventricular obstruction secondary to cerebellar tumor progression between enrollment and the three-week time point. In contrast, treated mice showed that IPI-926-induced tumor regression reduced the extent of hydrocephalus and minimized the extent of ventriculomegaly and transependymal CSF flow, contributing to the normal physical appearance (no prominent skull bulging) of treated mice. Nevertheless, despite neurological improvement, approximately half of the mice treated with 20 mg/kg/day IPI-926 exhibited a rebound in tumor growth by 6 weeks following maximal size reduction at the 3 week MRI time point (FIG. 18).

More specifically, MR scans of each mouse were performed at enrollment, after 3 weeks of daily IPI-926 treatment, and after 6 weeks of daily IPI-926 treatment. T2-weighted axial images were acquired at 3 Tesla, using a Philips MRI system with a custom mouse head coil. Vehicle treated mice were imaged in parallel, although no vehicle treated mice survived until the six-week imaging time point. A wild-type mouse was scanned as a reference. MR images demonstrate the enlarged ventricles and trans-ependymal cerebral spinal fluid (CSF) flow resulting from cerebellar tumor progression in a vehicle treated mouse (data not shown). MR images from IPI-926 treated mice demonstrate a significant reduction in ventricle size and a resolution of transependymal CSF flow, resulting from decreased tumor burden and a lesser degree of fourth ventricle obstruction. Histopathological evaluation at the final six-week time point validated the radiological findings, with a significant reduction in ventricle size evident in IPI-926 treated mice (see FIG. 17). Tumor volume was estimated from MR scans taken at enrollment, after 3 weeks of treatment and after 6 weeks of treatment. Analysis of tumor volume showed tumors initially receded in response to daily IPI-926 treatment, but this response was limited after three weeks. Graphs in FIG. 18 show estimated tumor volumes (mm³) at each time point for vehicle treated (n=5) and IPI-926 treated Ptc^{C/C} mice (n=7). Note that none of the vehicle-treated mice survived until the 6 week imaging time point.

Histopathological evaluation of vehicle treated mice showed distorted cerebellar architecture as a result of unencapsulated and infiltrative neoplastic growth, consisting of elongate, spindle-shaped cells with indistinct cell boundaries, abnormally shaped nuclei and stippled chromatin. In IPI-926-treated tumors, reduced tumor volume and a moderate reduction in tumor cell density were observed. More of the normal cerebellar architecture was visible in IPI-926 treated mice, along with multi-focal regions of malacia, necrosis and inflammation, which appear to be secondary to tumor cell death. The MRI and histological findings prompted two sets of experiments, one to assess the impact of maintenance treatment regimens on survival and the other to establish the mechanism(s) underlying disease progression during treatment.

IPI-926 Maintenance Administration Prolongs Survival in Mice Bearing Intracranial Medulloblastomas, while Continued IPI-926 Administration Induces Regression of Flank Allografts from Drug Resistant Donors

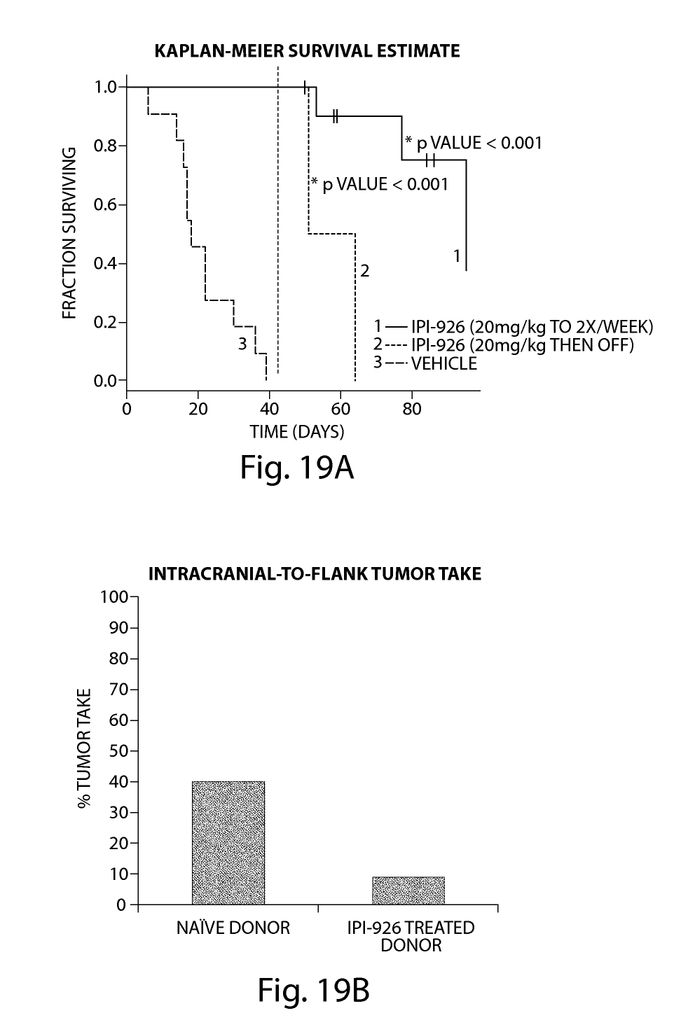

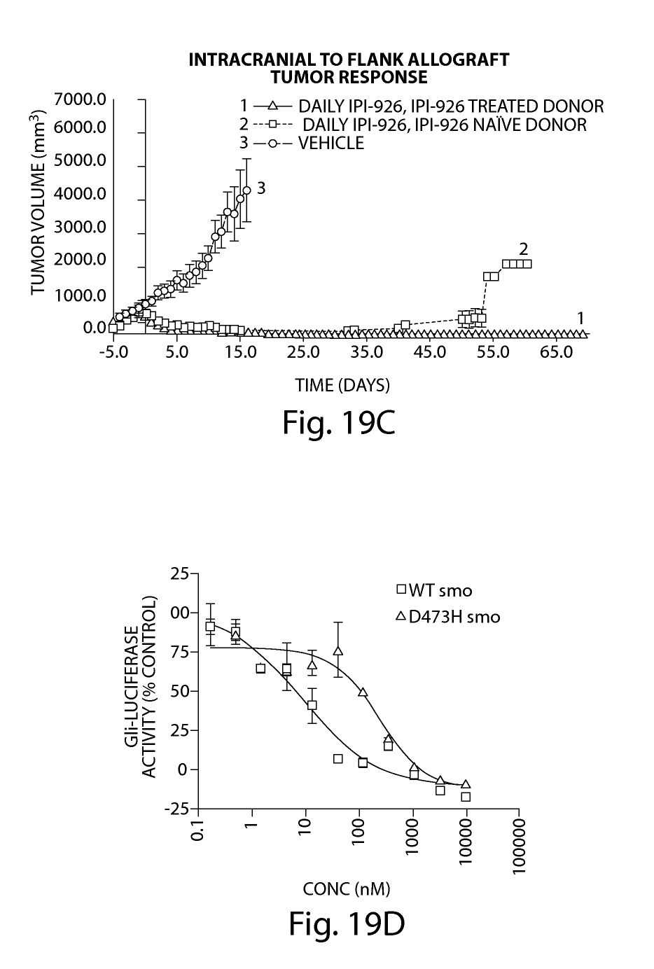

To further establish the extent to which IPI-926 can prolong survival, several dosing regimens in trials with overall survival as the primary endpoint were assessed. In one study, three- to five-week-old Ptc^{C/C} mice symptomatic for medulloblastoma were randomized to receive vehicle (line #3) or intraperitoneal IPI-926. Mice were initially given daily IPI-926 (20 mg/kg/dose) for six weeks (n=24), and were then taken off the drug (n=12; line #2) or given maintenance dosing (20 mg/kg twice per week) for six additional weeks (n=12; line #1; FIG. 19A). Tumors progressed rapidly after the withdrawal of drug following the initial six weeks of daily IPI-926 therapy (line #2) and mice died within an average of 10 days after stopping treatment (FIG. 19A). In contrast, 77% of mice receiving maintenance dosing (20 mg/kg IPI-926 twice per week, line #1) were still alive six weeks after starting twice-a-week therapy. Thus, continued IPI-926 treatment following six weeks of daily therapy prolonged median survival five-fold compared to vehicle treated control animals. Having established tumor regression, neurologic improvement, and a survival advantage conferred to Ptc^{C/C} mice, it was sought to determine whether tumor initiating capacity, which is important for metastases generation, was impaired by drug treatment.

### IPI-926 Reduces Medulloblastoma Tumor Initiating Capacity

Medulloblastoma cells from the Ptc^{C/C} mice have tumor initiating potential, as evidenced by their ability to form new tumors when transplanted to wild type recipient mice. To confirm this, aliquots of 1 million cells from the cerebellar tumors of 9 donor Ptc^{C/C} mice were transplanted to the flanks of 110 recipients. Tumors were established from 7 of 9 donors and a total of 40 of the 110 recipients grew flank tumors. In contrast, the same approach yielded tumors in only 9 of 51 recipients when donors were exposed to daily treatment with 20 mg/kg IPI-926 for 6 weeks prior to transplantation (FIG. 19B). Flank allografts were generated from either drug-naïve Ptc^{C/C} tumors or Ptc^{C/C} tumors from mice treated with IPI-926 for 6 weeks and the tumor take rates are shown in FIG. 19B (P values were generated using Fisher's exact test). This demonstrated that IPI-926 reduced tumor initiating potential in this aggressive medulloblastoma mouse model (P=0.017).

IPI-926 Induces Regression of Flank Allografts from Drug Refractory Donors

The flank allografts established from a donor treated with IPI-926 for 6 weeks prior to transplantation studies grew at approximately the same rate as tumors from drug naïve donors (FIG. 19C). Recipient mice bearing drug-naïve and IPI-926 treated allograft tumors were then treated with daily IPI-926 (20 mg/kg) and tumor growth was monitored via caliper measurements. The average tumor volumes are shown in FIG. 19C, with error bars representing +/−SEM. When tumor volumes reached 500 cm³, recipient mice then received either daily IPI-926 (20 mg/kg) or vehicle treatment, and the growth of flank allografts was monitored over a nine-week period. Surprisingly, daily IP administration of IPI-926 into recipient mice suppressed tumor growth to the point that tumors were undetectable by caliper measurements in 100% of both allograft groups (FIG. 19C). This experiment demonstrated that intracranial medulloblastomas treated with IPI-926 that re-grew during the course of initial treatment were responsive to the drug when implanted as sub-cutaneous allografts. Half of the allograft mice from drug-treated donors were taken off drug after five weeks of IPI-926 treatment, and the other half received continuous IPI-926 for the nine-week study period. Mice from both groups were monitored for the entire 9-week period. Only 1 of 6 tumors re-grew in the mice that went off drug following the five-week treatment period and no tumors re-grew in mice receiving continuous IPI-926. The response of flank tumors derived from the IPI-926-treated donor mouse was attributed to higher drug concentrations in the flank tumors compared to brain tumors, the latter of which are at least partially protected by the blood brain barrier (BBB). Consistent with this, IPI-926 concentrations were found to be 478±98 ng/g and 1,269±570 ng/g in cerebellar tumors of mice treated with 20 mg/kg/day IPI-926 for 4 or 42 days, respectively, whereas drug levels in flank tumors were 47,320±27,887 ng/g and 22,053±3834 ng/g, respectively, in mice treated with 7 days or 42 days of IPI-926 using the same dosing regimen (summarized in Table 3). While the markedly higher drug concentrations achieved in flank tumors were sufficient to overcome the drug tolerance observed in the cerebellar tumor of the donor mouse, the higher concentration alone was not sufficient to sustain remission in mice that received flank allografts from drug-naïve donors. Forty percent of these tumors progressed while on therapy during the 9-week trial despite initially disappearing in response to IPI-926 administration. However, given the aggressiveness of tumors from this model, IPI-926 still provided a significant benefit to treated animals.

FIG. 19D demonstrates the average Gli-luciferase reporter activity in C3H10T1/2 cells transfected with wild type SMOOTHENED (SMO) (squares) or the D473H SMO mutant (triangles) after treatment with various doses of IPI-926. Reporter activity is normalized to untreated C2H10T1/2 cells.

Escape from Shh Inhibition Accompanies Tumor Progression in IPI-926 Treated Mice

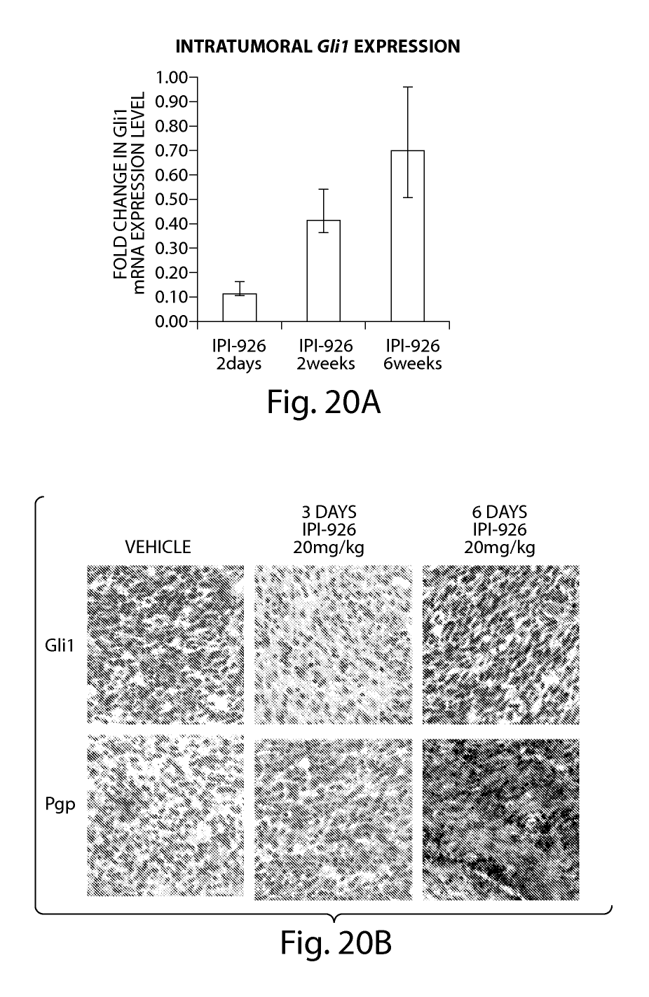

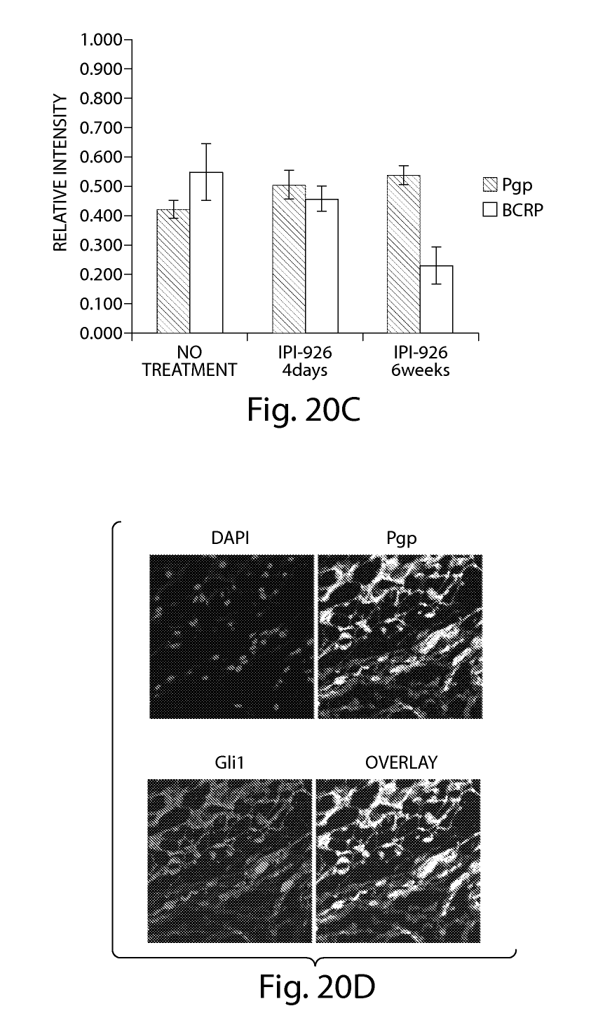

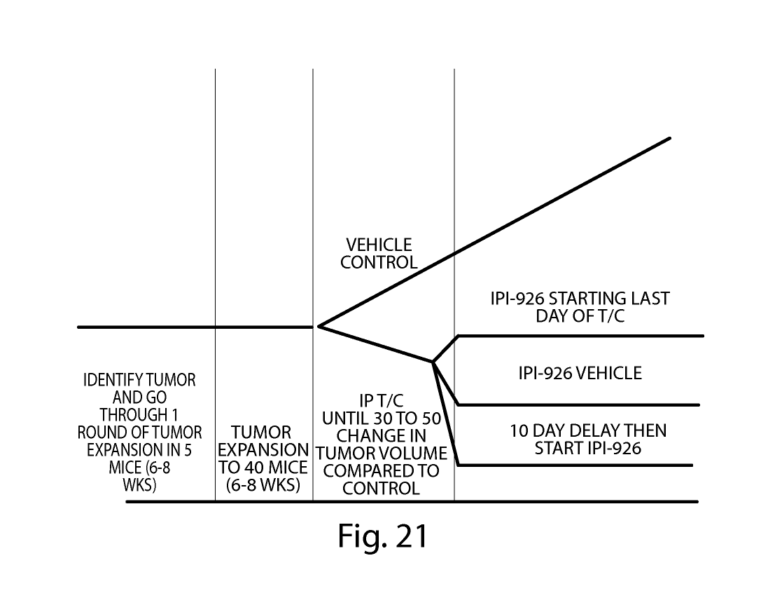

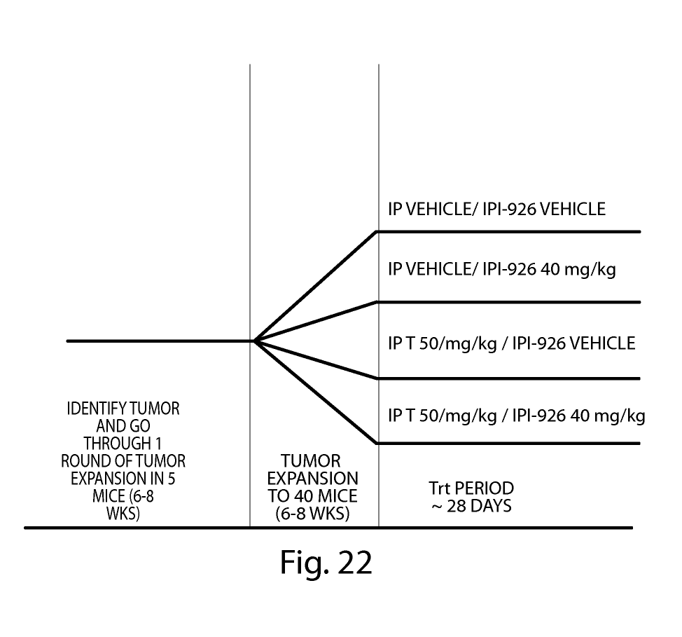

To better understand why tumors grew despite IPI-926 treatment, the extent to which Gli1 was inhibited by IPI-926 at the end of therapy compared to the beginning was assessed. Effective inhibition of Shh signaling with IPI-926 in tumors that grew during therapy would indicate that cells were adapting to drug by utilizing parallel signaling pathway(s). In contrast, reduced Gli1 suppression at the end of therapy would indicate that resistance was driven by such mechanisms as drug efflux pumps or genetic mutations that reduced IPI-926 affinity for Smoothened. The latter group of possibilities was supported by the observation that IPI-926 suppressed Gli1 levels by 90% after 2 days of therapy, by 60% after 2 weeks of therapy, and by 30% after 6 weeks of therapy compared to expression levels detected in brain tumors from vehicle treated controls (FIG. 20A). The pharmacodynamic activity of IPI-926 in Ptc^{C/C} tumors was confirmed by analysis of Gli1 mRNA by RT-PCR (FIG. 20A). The initial reduction in Gli1 expression seen in response to daily IPI-926 (20 mg/kg/dose) was diminished after 6 weeks of daily treatment. Bars represent the average fold change in Gli1 expression normalized to vehicle-treated controls using n=3 per group, with error bars representing +/−SEM.

Expression analysis was further confirmed by immunohistochemistry with an antibody recognizing Gli1. Immunohistochemistry with an antibody recognizing Gli1 also demonstrated that the initial decrease in Gli1 staining in response to IPI-926 was diminished in medulloblastomas treated daily over a 6-week period (Top panels, FIG. 20B). Tissue sections from mice treated daily with IPI-926 (20 mg/kg) for 3 days and 6 weeks were stained in parallel to tissue sections from vehicle-treated controls to analyze expression of Gli1 protein within the respective medulloblastomas. Images shown are at 40× magnification. The BBB has been shown to increasingly limit drug penetration into the brain over time through induction of drug efflux pumps (Losher et al., (2005)). This was not the case in the present study, as IPI-926 concentrations increased in cerebellar tumors over time (Table 3). This left development of drug resistance mutations or cancer cell drug efflux pumps as the remaining primary candidates responsible for tumor progression during monotherapy with IPI-926.

### Lack of Mutations Conferring Resistance to IPI-926

In principle, cancer cells could escape drug inhibition through mutations in the drug binding pockets. A previous mutagenesis study identified 8 mutations that activated the Smoothened protein, all of which were located in either the sixth (TM6) or seventh (TM7) transmembrane domains (Taipale et al., (2000) Nature 406:1005-1009). A recent study further demonstrated that treatment of Ptch1^{+/-}; p53^{-/-} flank allografts with the Hedgehog antagonist GDC-0449 resulted in resistance conferred by a heterozygous A-to-G missense mutation causing a D477G change, which maps to the C-terminal end of TM6 (Yauch et al. (2009) Science 326: 572-574). In contrast, tumors that grew despite ongoing IPI-926 therapy showed no evidence of mutations in TM6 or TM7. Of the 8 brain and 3 flank tumors from which the Smoothened gene was sequenced, only one showed sequence variations that could not be readily attributed to known inter-strain single nucleotide polymorphisms. A point mutation at Asparagine 223 was observed in a single flank allograft that re-grew despite continuous IPI-926 treatment. This site is not within any of the seven transmembrane domains within the Smoothened protein and does not map to a region of the protein with a known functional domain, or within proximity of any of the previously identified activating mutations. Given that all characterized activating Smoothened mutations localize to the TM6 and TM7 domains, and the substantial response of heavily treated tumors in the allograft setting, we conclude that it is unlikely that the re-growth of both intracranial and flank allografted medulloblastomas is dependent on de novo Smoothened mutations.

### IPI-926 Activity on D473H SMO Mutant

To determine the ability of IPI-926 to suppress Shh signaling in the context of the D473H SMOOTHENED (SMO) mutant known to confer resistance to the Shh pathway antagonist GDC-0449 (Yauch et al. (2009) Science 326: 572-574), the half maximal concentration (IC₅₀) of IPI-926 required to inhibit Gli-luciferase activity was measured (FIG. 19D). IPI-926 inhibited reporter activity at an IC₅₀ of 9 nM in C3H10T1/2 cells transfected with wild type SMO, but also showed activity against the D473H SMO mutant at an IC₅₀ of 244 nM. These findings are in contrast to results obtained with other hedgehog pathway antagonists, and indicate that IPI-926 retains the ability to impair downstream hedgehog signaling even in the presence of some activating SMO mutations.

### Drug Transporters in Ptc^{C/C} Medulloblastomas

One of the main mechanisms of drug resistance in cancer cells is aberrant expression of ATP-binding cassette (ABC) transporters, which utilize active transport to efflux drugs from treated cells. Many chemotherapeutic drugs currently used in the cancer treatment are substrates of the ABC transporters Pgp/ABCB1 and BCRP. To determine whether either of these was upregulated in response to IPI-926 therapy, Pgp and BCRP transporters were quantified via Western blotting in samples from untreated and IPI-926 treated mice. The expression levels of Pgp and BCRP were not significantly increased by daily treatment with IPI-926 for four days or six weeks (FIG. 20C). Because the Western analyses were done on tissue homogenates that include both normal and neoplastic cells, immunohistochemistry (IHC) studies were also performed to assess Pgp protein expression with cellular resolution. IHC staining revealed focal increases in Pgp within the medulloblastomas of mice that were treated for 6 weeks with IPI-926 (lower panels, FIG. 20B). Double immunostaining showed that Pgp staining was highest in cells that also stained brightly with an antibody that recognized Gli1 (FIG. 20D). This analysis revealed that the Shh pathway is preserved in the face of IPI-926 therapy in cells with Pgp protein expression levels in patterns that are readily detectable by IHC. Taken together with the efficacy studies described above, these data indicate that IPI-926 as a monotherapy induces very good or complete response in most Ptc^{C/C} mice and that efficacy is likely limited primarily by the same drug efflux mechanisms that limit most oncology drugs when used as single agents.

More specifically, expression of the ABC transporter pump Pgp is induced by prolonged IPI-926 treatment. Medulloblastoma-bearing Ptc^{C/C} mice were treated with daily IPI-926 (20 mg/kg) for 4 days or 6 weeks and tissue lysates generated from the remaining tumors and from untreated control tumors. The expression of Pgp and BCRP were analyzed via Western blot and normalized to a Beta-actin loading control (data not shown). The experiment was performed in triplicate and the resulting blots were quantified via imageJ program and the relative intensity is shown in FIG. 20C. In parallel, tissue sections from mice receiving daily IPI-926 (20 mg/kg) for 3 days or 6 weeks and vehicle controls were stained with antibodies recognizing Pgp (Lower panels, FIG. 20B) and BCRP (data not shown). Double immunofluorescence analysis revealed that most of the cells expressing Gli1 (in red) also express Pgp (in green), indicating that hedgehog pathway activity is maintained in cells with active ABC transporters (FIG. 20D).

Tumor response was monitoring tumor response via magnetic resonance imaging (MRI). MR scans in the sagittal plane from vehicle treated or IPI-926 treated Ptc^{C/C} mice were monitored at enrollment, after 3 weeks of daily IPI-926 treatment, and after 6 weeks of daily IPI-926 treatment, and after drug withdrawal. T2-weighted axial images were acquired at 3 Tesla, using a Philips MRI system with a custom mouse head coil. Control mice were imaged at enrollment and after 3 weeks on daily vehicle treatment, although no vehicle treated mice survived until the six-week imaging time point. Live animal images were evaluated in parallel for vehicle treated and IPI-926 treated mice.

### 5.4 Discussion

Medulloblastoma is an aggressive malignant brain cancer that is particularly difficult to cure in the recurrent disease setting. Conventional therapies for medulloblastoma impose unacceptable toxicities on children with this disease and more effective, less toxic alternatives are critical for the future care.

A recent clinical study reported a human patient with metastatic medulloblastoma that initially responded to the Shh antagonist GDC-0449 (Rudin et al. (2009) The New England Journal of Medicine 361:1173-1178). Unfortunately, the patient developed cell-autonomous resistance to the drug through de novo emergence of a clone with a point mutation in Smoothened that reduced the affinity to the drug binding site (Yauch et al. (2009) Science 326: 572-574). A similar mutation was observed in mice treated with GDC-0449 (Yauch et al. (2009) Science 326: 572-574). It is important to learn whether rapid emergence of mutation-based drug resistance is unique to certain small molecule Shh antagonists or is universal.

In this study, the efficacy of the novel Smo inhibitor IPI-926 against spontaneously-arising medulloblastoma in the conditional Ptc^{C/C} mouse model was analyzed. Treatment with IPI-926 was well tolerated and induced tumor regression and a significant survival benefit. Six weeks of daily IPI-926 at 20 mg/kg resulted in 100% survival in compared to 0% in the vehicle-treated mice. Additionally, a substantial resolution in clinical symptoms was observed in the majority of IPI-926 treated mice, secondary to reduced hydrocephalus, calvarial swelling and accompanied by increased mouse activity.

This study examined the effects of hedgehog pathway antagonism in the conditional Ptc^{C/C} mice. A previous study demonstrated the efficacy of the HhAntag hedgehog antagonist in a less aggressive Ptch1+/−; p53−/− model of medulloblastomas arising in the Ptch1 heterozygous, p53 null background (Romer et al. (2004) Cancer Cell 6: 229-240). A substantial decrease in tumor mass following 2 weeks of twice daily treatment with 20 mg/kg or 100 mg/kg HhAntag was observed in mice that were enrolled at 3 weeks of age. A survival benefit was also noted in an extended study of mice enrolled at 5 weeks of age and treated with 100 mg/kg HhAntag in comparison to vehicle-treated controls. While these results share similarities to the current study, an important contrast must be noted in the extent of tumor burden in response to heterozygous versus homozygous loss of Patched1 within the cerebellum. In the Ptc^{C/C} model, all cerebellar granule neuron precursor cells are lacking the inhibition normally mediated by the Patched1 receptor, and tumor formation is early, aggressive and uniform throughout the cerebellum. In contrast, tumors from the Ptch1 heterozygous background are initially more focal and possess substantially more normal cerebellar architecture, despite p53 deficiency. In our study, Ptc^{C/C} were randomized to receive either vehicle or IPI-926 treatment after they were clinically symptomatic, which occurs between three and five weeks of age and is the result of substantial effacement and extensive tumor burden. Thus, the response to IPI-926 was remarkable given the continuous source of neoplastic cells and the extent of initial tumor burden in intracranial Ptc^{C/C} tumors.

The heptahelical structure of the Smoothened receptor is required for binding of cyclopamine and is targeted by G protein coupled receptor modulators (Chen et al., (2002) Genes & Development 16: 2743-2748; Goudet et al., (2004) Drug Discovery Today 1: 125-133), and mutations near the highly conserved transmembrane domains can reduce the affinity of compounds specifically targeted to this binding pocket. In contrast to the previous report of mutation-based resistance to GDC-0449, no mutations in the TM6 or TM7 domains of the Smoothened allele were observed. In all but one tumor, no mutations were observed aside from the SNPs expected in mice on a mixed strain background.

Like most oncology drugs, including previously reported Shh antagonists, IPI-926 is a Pgp substrate. IHC studies revealed that elevated Gli1 levels in cells of heavily treated medulloblastomas co-localize with high Pgp expression. This suggests that Pgp can be partially responsible for providing a survival advantage to cells that retain Shh activity in the face of IPI-926 therapy. It initially seemed paradoxical that IPI-926 concentrations were higher, rather than lower, in tumors that had been exposed to IPI-926 for 6 weeks. The traditional portrayal of drug efflux pump mechanisms would suggest that drug levels in tumors should be reduced rather than elevated. However, overexpression of ABC transporters can confer drug resistance to cancer cells by modifying the intracellular drug distribution through at least two different mechanisms (Larsen et al., (2000) Pharmacology & Therapeutics 85: 217-229). ABC transporters expressed in the plasma membrane mediate drug resistance by decreasing total intracellular drug accumulation. ABC transporters localized in intracellular membranes can decrease the drug accessibility to its target by intravesicular accumulation of drug, which could occur via sequestration into intracellular organelles (Larsen et al., (2000) Pharmacology & Therapeutics 85: 217-229; Ifergan et al., (2005) Cancer Research 65: 10952-10958). These mechanisms would explain the failure to respond to IPI-926 despite the high drug concentrations found in tumors. Unfortunately, attempts to improve oncology drug performance by co-administration of anti-cancer drugs with compounds that block Pgp or other drug resistance proteins have not yet been successful. Hence, oncologists continue the strategy of achieving rapid tumor mass reduction through the combination of multiple effective drugs that have minimal overlapping toxicity to reduce the tumor initiating potential of residual cancer cells.

To our knowledge, no oncology drugs, including cytotoxic chemotherapy agents, have been shown to increase survival 5-fold in mice with advanced, aggressive, autochthonous brain tumors. Like other Shh antagonists used for extended periods in human and mouse studies (Olive et al., (2009) Science 324: 1457-1461; Von Hoff et al. (2009) N. Engl. J. Med. 361: 1164-1172), IPI-926 therapy was well tolerated by mice, including those that received daily therapy with 20 mg/kg drug for greater than 60 days and those that received once-weekly treatments of 70 mg/kg (not shown). These results further support that drugs specifically targeting the hedgehog pathway could be well tolerated individually and as part of a combined regimen. Studies that are currently underway in pediatric patients and those being planned must consider the permanent changes on cartilage and bone formation observed in young mice as a result of treatment with the HhAntag Smoothened inhibitor (Kimura et al., (2008) Cancer Cell 13: 249-260). The extent to which this on-target toxicity is species specific remains unknown at this time.

In summary, the results shown herein demonstrate the efficacy of IPI-926 in resolving clinical symptoms of advanced medulloblastoma and prolonging survival in the Ptc^{C/C} model. These data also provide additional evidence that this class of signal transduction pathway inhibitors should be further evaluated for their potential to improve outcomes in sonic hedgehog-driven tumors.

### Example 6

### Effects of IPI-926 in Reducing Ovarian Tumor Growth and Recurrence in a Xenograft Model

### 6.1 Background:

Epithelial ovarian cancer is the second most common, but most lethal gynecologic malignancy in the United States and was estimated to affect over 20,000 women with more than 16,000 deaths in the USA in 2008 (Jemal A, et al. (2009) CA Cancer J Clin 59(4):225-249). No effective screening strategy has been determined, thus the majority of women present with advanced stage disease. At the time of diagnosis, women undergo aggressive surgical cytoreductive surgery with the subsequent delivery of platinum based therapy. The combination of carboplatin and paclitaxel is the standard first line combination in the US. The position of platinum and taxane based therapy has been consolidated with the use of intraperitoneal therapy with a significant survival benefit in prospective randomized clinical trials (Ozols R F, et al. (2003) J Clin Oncol 21(17):3194-3200). This therapy, while effective at generating responses in 70-80% of women and clinical remissions in half, is seldom curative. Despite advances in therapy and delivery, recurrence and chemotherapy resistance are still formidable problems as the majority of patients with ovarian cancer who achieve a complete remission with first line platinum-based chemotherapy typically ultimately develop recurrent disease.

Residual tumor is believed to contain a tumor initiating cell (TIC) population that is more resistant to current chemotherapies. The hypothesis is based, in part, on the belief that the putative TICs have undergone one or more mutations in genes regulating self renewal (Al-Hajj M & Clarke M F (2004) Oncogene 23(43):7274-7282). The most well recognized signaling pathways regulating self-renewal in benign cells would include but are not limited to the Hedgehog (Hh), β catenin/WNT, and Notch signaling pathways. All of these pathways have been implicated in the development and/or pathology of cancer (Takahashi-Yanaga F & Kahn M (2010) Clin Cancer Res 16(12):3153-3162; Merchant A A & Matsui W (2010) Clin Cancer Res 16(12):3130-3140).

Recent investigations have suggested that the Hh signaling pathway plays an important role in ovarian cancer pathogenesis. The majority of the data suggest that Hh signaling is up-regulated in epithelial ovarian carcinoma cell lines and cell line derived xenograft tumors (Bhattacharya R, et al. (2008) Clin Cancer Res 14(23):7659-7666). Through the use of Hh pathway antagonists like cyclopamine, a Smoothened inhibitor, investigators have shown that ovarian carcinoma cell line proliferation and xenograft growth are markedly impaired further supporting a role for Hh signaling in ovarian carcinoma (Chen X, et al. (2007) Cancer Sci 98(1):68-76). An association between Patched and Gli1 over expression with poor survival of ovarian cancer patients has also been demonstrated (Liao X, et al. (2009) Carcinogenesis 30(1):131-140). The focus of this study is to further elucidate how the Hh signaling pathway contributes to the pathogenesis of ovarian cancer and can be used as a targeted therapy.

A serial transplantation model was developed in which primary tumors from ovarian cancer patients are grown in NOD/SCID mice while maintaining their pathologic characteristics. This xenograft model was used to demonstrate that human tumors hosted in these mice did in fact contain a sub-population of cells which have the capacity for self-renewal allowing for successive re-initiation of tumor formation (Curley M D, et al. (2009) Stem Cells 27(12):2875-2883). The consecutive serial transplantation of primary human ovarian tumor cells in these mice resulted in decreasing time to tumor formation with each successive transplant, indicating that this system is an efficient platform for carrying out enrichment experiments in vivo. Moreover, the generation of serially transplantable tumors indicates the presence of a self-renewing stem cell-like population. Unlike most pre-clinical studies that utilize cell-lines to generate mouse xenografts, the explants of the present study are generated from primary tumors and can be a more accurate model of clinical patient tumors. Furthermore, using this primary tumor model, the limitations of using cell lines that have been exposed to years of culture can be bypassed. This model has already been pivotal in demonstrating the efficacy of IPI-926 in ovarian cancer.

### Objectives:

One objective is to expand previous studies to further investigate the conditions that IPI-926 is most effective in inhibiting growth of human serous ovarian cancer xenografts. More specifically, the study will determine and/or identify whether there is a critical window with which IPI-926 must be administered to be effective as a consolidative therapy. Secondly, it will be determined if IPI-926 is effective as a single agent or as an adjunct therapy in platinum resistant tumors. The specific experiments proposed are designed to address the following hypotheses.

### Testing

1) Tests can be performed to determine the optimal time for initiation of IPI-926 treatment. The time of administration of IPI-926 post primary chemotherapy will be important to determine its effectiveness in a consolidation setting. Delayed administration of IPI-926 until the residual chemotherapy is diminished can reduce its effectiveness and can not inhibit recurrent disease.
2) IPI-926 can be effective in platinum resistant disease either as a single agent or in combination with paclitaxel.

### Study Design and Results:

Excess ovarian tumor tissue from patients was collected. Histologically confirmed papillary serous ovarian tumors was disaggregated into purified tumor cells devoid of hematologic components. These cells were suspended in a 1:1 PBS:Matrigel®. A suspension of a specified number of cells was injected subcutaneously (SC) into 6 week old NOD/SCID mice (NOD/LtSz-Prkdcscid/J; 6-8 weeks; Jackson Labs). The mice were housed and maintained in accordance with the institutional guidelines and tumor formation in the injected animals is monitored regularly. Subcutaneous tumors were measured weekly with calipers, and the volume (in mm³) was determined using the formula: [length (mm)×width (mm)×width (mm)]/2. Animals were euthanized when they become moribund or had evident excessive tumor burden. For continued propagation in mice, the generated tumors were excised and processed as described for the primary tumor samples, depleted of mouse H2 Kd+ cells (MACS beads) and re-injected subcutaneously into new recipient NOD/SCID mice. All tumor cells utilized for these experiments underwent at least 3 passages to ensure the presence of a tumor initiating population. Histology of each generation was evaluated to confirm the maintenance of papillary serous histology.

These experiments were tiered to investigate the pharmacodynamic properties of IPI-926 along with its efficacy and synergy with conventional therapy. The expression of various Hh pathway targets was evaluated, both at the mRNA and protein level in the pre-treated and treated serous ovarian tumor samples.

Following serial transplantation, a minimum of 40 tumor-bearing mice (300-600 mm³) were treated with vehicle or paclitaxel (15 mg/kg) and carboplatinum (50 mg/kg) (T/C) IP q 7 days. Once the tumor volume in the T/C arm was reduced in mass by a minimum of 30% of their original volume at the start of treatment, the mice in the vehicle arm were harvested. The remaining mice bearing matched sized tumors were randomized into one of three groups. The first group received IPI-926 (40 mg/kg) by gastric lavage beginning on the last day of T/C and continued every other day for at 4-6 weeks. The second group received IPI-926 (40 mg/kg) by gastric lavage beginning 10 day post T/C treatment and continuing every other day for 4-6 weeks (minus the washout time). The last arm consisted of mice receiving vehicle alone beginning on the last day of T/C treatment and continuing until the end of the experiment. Tumor volume and mouse weights were regularly assessed. The experiment was performed in triplicate with at least three separate patient-derived serous ovarian tumors for validity.

The endpoints measured included mouse weights, tumor volume, and tumor weights post harvest. Sub samples of tumor were collected, processed for H&E, IHC and nucleotide analysis. RT PCR was used to assess expression of mouse and human Gli-1 and SHh tumor explants after treatment. IHC for Gli and SHh was also performed with an appropriate IgG control.

### Platinum Resistant Disease

### Study Design:

In this experiment, the adjuvant activity of IPI-926 in a platinum resistant setting is assessed. Platinum resistance is based on the original patients clinical diagnosis and confirmed in an in vivo setting. If necessary, mice hosting tumor explants are treated with the standard T/C regimen and generate platinum resistant tumors. Mice bearing matched sized tumors (300-600 mm³) are randomized into one of four groups receiving IPI-926 40 mg/kg PO q 7 days along with intraperitoneal (IP) vehicle; or paclitaxel (15 mg/kg) T) IP q 7 days with oral vehicle; IPI-926 40 mg/kg q 7 days+IP T; or oral vehicle q 7 days+IP vehicle q 7 days. The adjuvant treatment period spans approximately 28 days. Tumor volume and mouse weights is regularly assessed every three days.

The experiment is performed in triplicate with at least three separate patient-derived serous ovarian tumors/cells for validity. The number of tumors analyzed can increase in order to obtain appropriate representation of samples that have evidence of platinum resistance. Alternatively, a mouse model can be induced using mice hosting tumors treated with sub lethal concentrations of T/C, which will likely result in a platinum resistant phenotype.

RT-PCR is used to assess expression of mouse and human Gli-1 and SHh tumor explants after treatment. IHC for Gli and SHh is performed with an appropriate IgG control.

### Statistical Methods:

Non-parametric statistical analysis using Wilcoxan rank-sum tests for unpaired and sign-rank tests for paired data on tumor volumes and weights, as well as mouse weights will be performed. A P value of <0.05 will be considered to be statistically significant. STATA (College Station, Tex.) v10 software will be used for all tests.

### Example 7

### Hedgehog Inhibition Reduces Tumor Re-Growth Post-Cytoreduction in Multiple Preclinical Models of Minimal Residual Disease

This Example consolidates some of the data presented in previous examples demonstrating that in multiple pre-clinical models of MRD, IPI-926 shows anti-tumor activity post cytoreduction with either standard of care chemotherapy or targeted therapy. Taken together, these data suggest that the administration of IPI-926 post cytoreductive therapy can be used as a treatment option.

Minimal residual disease (MRD) is the presence of residual malignant cells after primary treatment (e.g., chemotherapy, radiation therapy, surgery, and targeted therapy), and in most cases, there are so few cancer cells present that they cannot be found by routine means. Importantly, in many instances the presence of these residual tumor cells eventually leads to disease recurrence and shortened survival.

IPI-926 is a potent and selective Hedgehog pathway antagonist that binds and inhibits the key signaling membrane protein Smoothened. In a phase 1 clinical trial, IPI-926 has been shown to be well-tolerated and has demonstrated clinical activity. IPI-926 is currently in two phase 2 trials, in pancreatic cancer in combination with gemcitabine, and in chondrosarcoma as a single agent.

In this Example, we demonstrate that in multiple pre-clinical models of MRD, IPI-926 shows anti-tumor activity post cytoreduction with either standard of care chemotherapy or targeted therapy. Taken together, these data suggest that the administration of IPI-926 post cytoreductive therapy can be used as a treatment option.

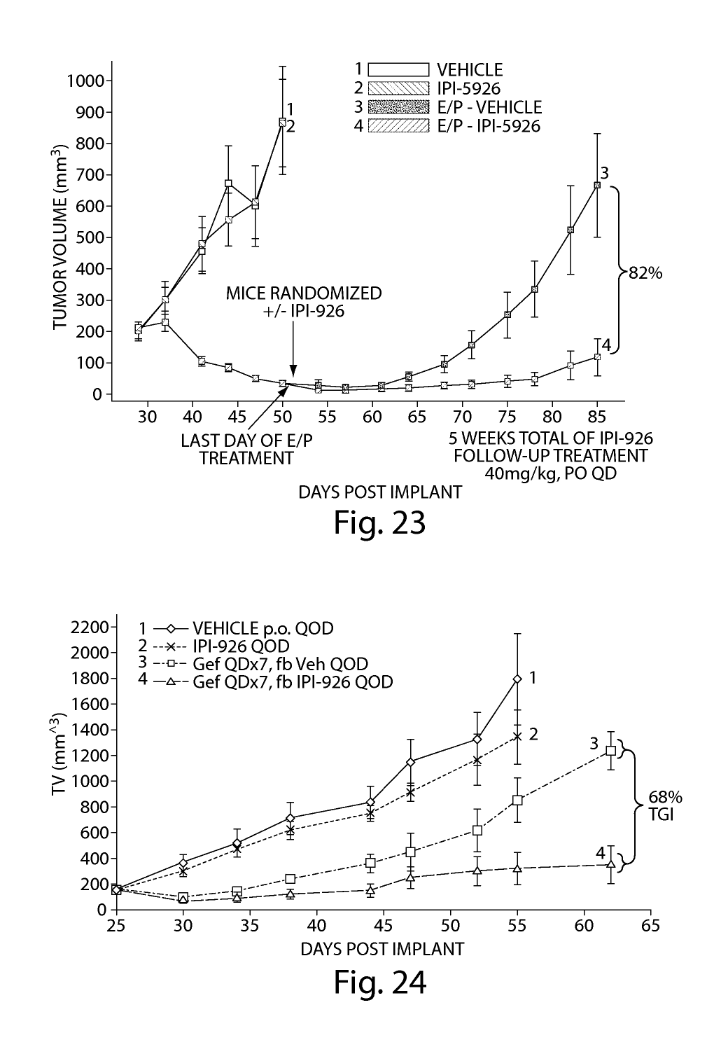

FIG. 23 is a linear graph showing the effect of IPI-926 on post tumor debulking in a primary xenograft model of SCLC. Tumors were established and treated with etoposide/cisplatin followed by vehicle or IPI-926. Similar results are described in Example 2, above. Thus, IPI-926 is shown to be efficacious post-chemotherapy in a primary SCLC model of MRD.

FIG. 24 is a linear graph showing the effect of IPI-926 on post tumor debulking in a xenograft model of mutant EGFR NSCLC. Tumors were established and treated with gefitinib followed by vehicle or IPI-926. Similar results are described in Example 3, above. Thus, IPI-926 is shown to be efficacious post-tyrosine kinase inhibition (TKI) in a mutant EGFR NSCLC model of MRD.

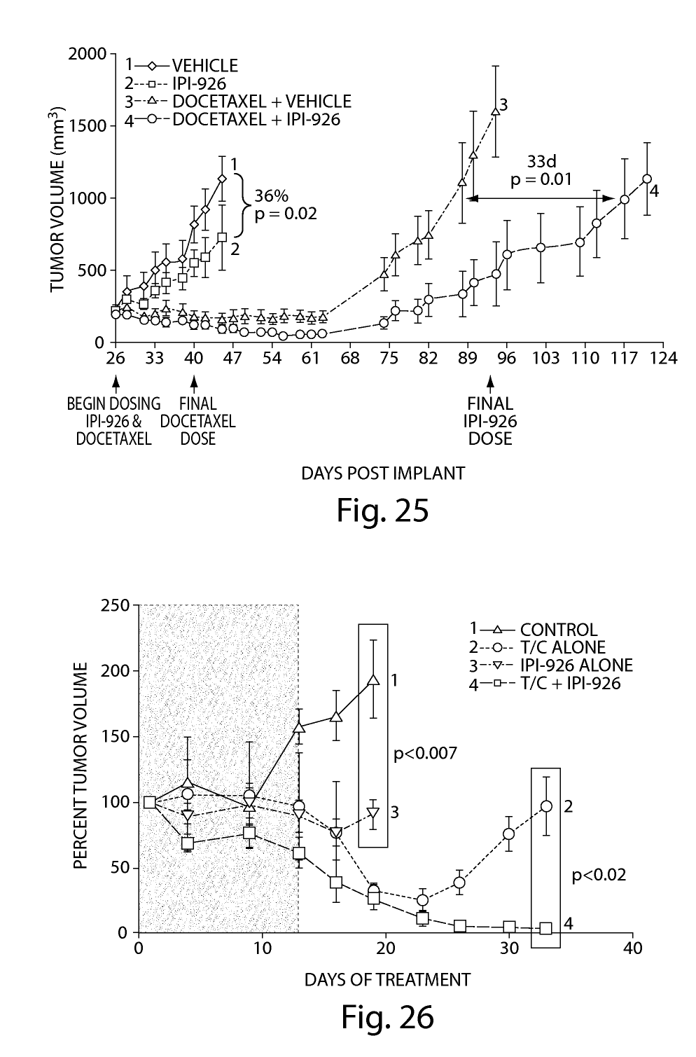

FIG. 25 is a linear graph showing the effect of IPI-926 on post tumor debulking in a primary xenograft model of castrate-resistant prostate cancer. Tumors were established and treated with docetaxel followed by vehicle or IPI-926. Similar results are described in Example 3, above. Thus, IPI-926 is shown to be efficacious post-chemotherapy in an MRD model of castrate-resistant prostate cancer.

FIG. 26 shows that mice treated with IPI-926 alone had a smaller percent tumor volume (p<0.007) compared to control treated mice after 20 days of treatment, indicating that IPI-926 is efficacious in the treatment of serous ovarian cancer. Mice were also treated with taxol/carboplatin followed by treatment with vehicle or IPI-926. FIG. 23 shows that mice treated with taxol/carboplatin followed by IPI-926 had a smaller percent tumor volume (p<0.02) than mice treated with taxol/carboplatin followed by vehicle control. These data indicate that IPI-926 displays efficacy post-chemotherapy in a model of minimal residual disease in primary serous ovarian cancer.

The expression of Gli1 was also determined in the stroma from serous ovarian cancer patients. The tumor-associated stroma was dissected from tumor samples of 19 patients with high grade serous ovarian cancer and then qRT-PCR was utilized to assess Gli1 levels. FIG. 27 shows that elevated Gli1 expression in stroma from serous ovarian cancer patients is associated with worsened survival (p<0.015).

In conclusion, IPI-926 administration post tumor debulking results in tumor re-growth inhibition in multiple pre-clinical models of MRD. Gli-1 expression correlates with worsened outcome in microdissected tumor stroma from serous ovarian cancer patient samples. Taken together these results demonstrate that IPI-926 intervention post cytoreductive therapy is a viable treatment option.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for treating a hedgehog-associated cancer, comprising:
administering a hedgehog inhibitor to a subject who is undergoing or who has undergone a primary cancer therapy, in an amount sufficient to treat the cancer, wherein the hedgehog inhibitor is administered at a treatment interval chosen from:
(i) after initiation, but prior to cessation, of the cancer therapy;
(ii) less than 7 days after cessation of the cancer therapy;
(iii) as maintenance therapy;
(iv) at a diminished dose from a first-line therapeutic dose; or
(v) prior to detection of a metastatic lesion,
thereby treating the cancer.

2. A method for reducing a minimal residual disease or tumor, comprising:
administering a hedgehog inhibitor to a subject who is undergoing or who has undergone a primary cancer therapy, in an amount sufficient to treat the cancer, wherein the hedgehog inhibitor is administered at a treatment interval chosen from:
(i) after initiation, but prior to cessation, of the cancer therapy;
(ii) less than 7 days after cessation of the cancer therapy;
(iii) as maintenance therapy; or
(iv) at a diminished dose from a first-line therapeutic dose,
thereby reducing the minimal residual disease or tumor in the subject.

3. The method of claim 1, wherein the hedgehog inhibitor in (i) is administered at least 1, 2, 3, 4, 5, 6, 7, 10, 14, or 20 days prior to cessation of the cancer therapy.

4. The method of claim 1, wherein the hedgehog inhibitor in (ii) is administered less than 144, 120, 100, 90, 72, 60, 48, 36, 24, 14, 10, 9, 8, 7, 6, 5, 4, 3, 2, or 1 hour after cessation of the cancer therapy.

5. The method of claim 1, wherein the hedgehog inhibitor in (iii) or (iv) is administered at a dose that is less than 90% the first line therapeutic dose.

6. The method of claim 1, wherein the administration of the hedgehog inhibitor delays tumor recurrence by at least 6 months compared to an untreated subject.

7. The method of claim 1, wherein the maintenance therapy is continued for six months or longer.

8. The method of claim 1, wherein the hedgehog inhibitor is administered to the subject chronically as a single agent.

9. The method of any of claims 1-5, wherein the hedgehog-associated cancer or minimal residual disease is chosen from one or more of: lung cancer, pancreatic cancer, prostate cancer, bladder cancer, ovarian cancer, breast cancer, colon cancer, liver cancer, myelofibrotic cancer, medulloblastoma, multiple myeloma, acute myelogenous leukemia (AML), chronic myelogenous leukemia (CML), acute lymphocytic leukemia (ALL), or neuroendocrine cancer.

10. The method of claim 1, wherein the hedgehog inhibitor is a compound having the following formula: 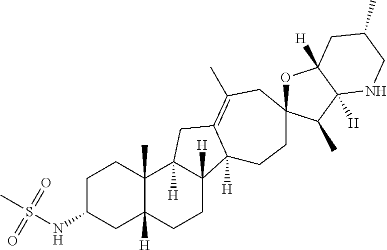
or a pharmaceutically acceptable salt thereof.

11. The method of claim 9, wherein the hedgehog inhibitor is a compound having the following formula: 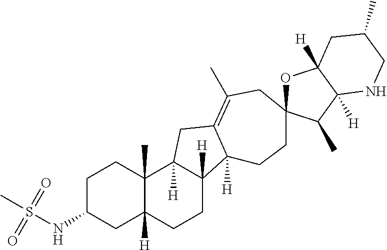
or a pharmaceutically acceptable salt thereof.

12. The method of claim 10, wherein the primary cancer therapy comprises one or more of an anti-cancer agent, surgery or radiation.

13. The method of claim 12, wherein the anti-cancer agent is chosen from one or more of: a tyrosine kinase inhibitor, a taxane, gemcitabine, cisplatin, epirubicin, 5-fluorouracil, a VEGF inhibitor, leucovorin, oxaplatin, cytarabine (Ara-C), an insulin-like growth factor receptor (IGF-1R) inhibitor, a PI3K inhibitor, an HSP90 inhibitor, folfirinox, a BRAF inhibitor, a MEK inhibitor, a JAK2 inhibitor.

14. The method of claim 1, wherein the subject is chosen from one or more of:
a patient with SCLC previously treated with a primary cancer therapy comprising etoposide and cisplatin;
a patient with NSCLC previously treated with a tyrosine kinase inhibitor; or
a patient with ovarian cancer previously treated with a taxol and/or carboplatin.

15. The method of claim 1, wherein the subject is a cancer patient substantially or completely in remission from one or more of: lung cancer, pancreatic cancer, prostate cancer, bladder cancer, ovarian cancer, breast cancer, colon cancer, liver cancer, myelofibrotic cancer, medulloblastoma, multiple myeloma, acute myelogenous leukemia (AML), chronic myelogenous leukemia (CML), acute lymphocytic leukemia (ALL), or neuroendocrine cancer.

16. A method for treating or preventing metastasis of a hedgehog-associated cancer, comprising administering to a subject in need thereof a hedgehog inhibitor prior to detection of a metastatic lesion, in an amount sufficient to inhibit or reduce a metastatic growth, thereby treating or preventing metastasis.

17. A method for treating a hedgehog-associated cancer that is partially or completely resistant to a primary cancer therapy, comprising administering to a subject in need thereof a hedgehog inhibitor in an amount sufficient to treat the cancer.

18. The method of claim 17, wherein the cancer harbors a mutation that renders the cancer resistant to a hedgehog inhibitor.

19. The method of claim 17, wherein the cancer has increased expression or activity of the PI3K pathway.

20. The method of either of claims 18-19, wherein the hedgehog inhibitor is a compound having the following structure: 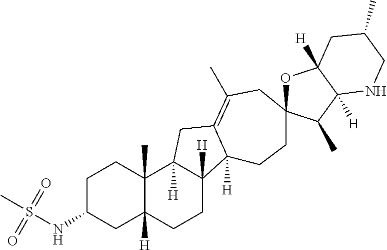
or a pharmaceutically acceptable salt thereof.

21. The method of claim 20, wherein the compound is administered in combination with GDC-0449.

22. The method of claim 20, wherein the hedgehog inhibitor is administered in combination with a PI3K inhibitor.

23. The method of claim 20, further comprising one or more of:
(i) analyzing a nucleic acid or protein from the subject chosen from a hedgehog ligand, a nucleic acid encoding a hedgehog ligand, or an upstream or downstream component of the hedgehog signaling;
(ii) evaluating a sample from the tumor, the cancer cell or the subject to detect the presence or absence of an alteration in an EGFR gene or gene product;
(iii) detecting the presence of one or more mutations in a hedgehog receptor; or
(iv) monitoring the subject for a change in one or more of: tumor size; hedgehog levels or signaling; stromal activation; levels of one or more cancer markers; the rate of appearance of new lesions; the appearance of new disease-related symptoms; the size of soft tissue mass; quality of life; or any other parameter related to clinical outcome.

24. A treatment regimen for use to treat, prevent, and/or reduce or inhibit the growth or re-growth of one or more hedgehog-associated cancers, the metastatic growth, and/or provide the minimal residual disease therapy and/or maintenance therapy, of claim 1.
